(19) United States
(12) Patent Application Publication
(10) Pub. No.: US 2026/0167736 A1
(43) Pub. Date: Jun. 18, 2026

(54) SUBCUTANEOUS DOSING OF ANTI-CD38 ANTIBODIES FOR TREATING PATIENTS WITH MODERATE TO SEVERE SYSTEMIC LUPUS ERYTHEMATOSUS

(71) Applicant: Takeda Pharmaceutical Company Limited, Osaka (JP)

(72) Inventors: Kristina Allikmets, Belmont, MA (US); Deborah T. Berg, Derry, NH (US); Eric Fedyk, Sudbury, MA (US)

(21) Appl. No.: 19/109,535

(22) PCT Filed: Aug. 31, 2023
(86) PCT No.: PCT/US2023/073238

§ 371 Date: Mar. 6, 2025

Related U.S. Application Data
(60) Provisional application No. 63/375,197, filed on Sep. 9, 2022.
(60) Provisional application No. 63/381,128, filed on Oct. 26, 2022.
(60) Provisional application No. 63/382,275, filed on Nov. 3, 2022.
(60) Provisional application No. 63/519,535, filed on Aug. 14, 2023.

(51) Int. Cl. C07K 16/28; A61K 31/195; A61K 31/343; A61K 31/4706; A61K 31/5377; A61K 31/573; A61K 39/00; A61P 37/06

(52) U.S. Cl. CPC C07K 16/2896; A61K 31/195; A61K 31/343; A61K 31/4706; A61K 31/5377; A61K 31/573; A61P 37/06; A61K 2039/505; A61K 2039/545; C07K 2317/41; C07K 2317/52; C07K 2317/565; C07K 2317/92

(57) ABSTRACT

Methods of treating patients with moderate to severe systemic lupus erythematosus (SLE) by administering isolated anti-CD38 antibodies subcutaneously are disclosed. Also disclosed are unit dosage forms for the anti-CD38 antibodies used in treating patients with moderate to severe SLE.

### CROSS REFERENCE TO RELATED APPLICATIONS

This application claims priority under 35 U.S.C. § 119(e) to U.S. Provisional Application Ser. No. 63/375,197 filed on Sep. 9, 2022, U.S. Provisional Application Ser. No. 63/381,128 filed on Oct. 26, 2022, U.S. Provisional Application Ser. No. 63/382,275 filed on Nov. 3, 2022, and U.S. Provisional Application Ser. No. 63/519,535 filed on Aug. 14, 2023, the entire disclosures of which are incorporated herein by reference.

### INCORPORATION BY REFERENCE OF MATERIAL SUBMITTED ELECTRONICALLY

The instant application contains a Sequence Listing which has been submitted electronically in XML file format and is hereby incorporated by reference in its entirety. Said XML file, created on Aug. 31, 2023, is named 101588-5016-WO Sequence Listing.xml and is 16,384 bytes in size.

### FIELD OF THE INVENTION

Methods for treating patients with moderate to severe systemic lupus erythematosus (SLE) by subcutaneously administering isolated anti-CD38 antibodies are disclosed. Also disclosed are unit dosage forms for the anti-CD38 antibodies used in treating patients with moderate to severe SLE.

### BACKGROUND OF THE INVENTION

Systemic lupus erythematosus (SLE) is a serious autoimmune rheumatic disease that has defied efforts to develop effective therapies, leaving a significant unmet medical need for patients with moderate to severe disease.

SLE is a heterogenous autoimmune disease characterized by dysregulation of T and B lineage cells, as well as other components of the innate immune system (Li et al. (2022) J. Autoimmun. 132: p. 102870 and Ma et al. (2019) Int. J. Mol. Sci. 20(23); incorporated by reference herein in their entireties). A hallmark of the disease is the production of pathogenic autoantibodies to double-stranded DNA (dsDNA), phospholipids, blood cells, and other targets (Dema, B. and N. Charles. (2016) Antibodies (Basel). 5(1); incorporated by reference herein in its entirety). Tissue damage in SLE is caused primarily by these pathogenic autoantibodies through immune complex deposition with Fc- and complement-mediated inflammation, as well as through direct antibody-target interactions. Virtually any organ or system in the body can be affected by SLE.

The clinical course of SLE is episodic, with flares of disease activity that lead to increased disability and organ damage over time. For moderate to severe cases, the current standard of care includes off-label therapies, including immunosuppressants (e.g., azathioprine, cyclophosphamide, and mycophenolate mofetil), antimalarials (e.g., hydroxychloroquine and chloroquine phosphate), corticosteroids (e.g., prednisone), and high-dose intravenous immunoglobulin (IVIg).

The therapeutic agents that are used for treatment of SLE have demonstrated limited success in targeting autoantibody production. Short-lived plasmablasts (PBs), plasma cells (i.e., terminally differentiated B cells), and long-lived plasma cells produce the characteristic pathogenic autoantibodies and are, therefore, critically involved in SLE pathogenesis. Studies have shown an increased number of PBs in the blood of patients with active SLE (Dorner et al. (2011) Arthritis. Res. Ther. 13(5): p. 243; incorporated herein by reference in its entirety).

To avoid the toxicity associated with general immunosuppression, trials have been developed using biologics aimed at selective B-cell depletion. Most of these trials, however, have given disappointing results (Cinquina et al. (2022) Curr. Rheumatol. Res. 3(1):1-3); incorporated herein by reference in its entirety). These failures, which may be partially attributed to heterogeneity in the pathophysiology of SLE, indicate the need for the development of more effective targeted therapeutics.

CD38 is a type II glycoprotein that is highly and uniformly expressed on antibody-producing PBs and plasma cells, making it a potential target for treatment of SLE (Parodis et al. (2022) Front. Med. (Lausanne). 9: p. 952304; incorporated herein by reference in its entirety).

In an ex vivo study of CD38 expression on various immune cells in peripheral blood mononuclear cells from patients with SLE, the highest CD38 expression was observed on plasma cells and PBs, followed by natural killer (NK) cells, plasmacytoid dendritic cells, a regulatory T cell subpopulation, and naïve T cells (Burns et al. (2021) Int. J. Mol. Sci. 22(5); incorporated herein by reference in its entirety).

Other studies have confirmed that the levels of CD38-expressing PBs are increased in patients with SLE (Clemens et al. (2017) Clin. Pharmacokinet. 56(8): 915-24; Sanada et al. (2016) Blood 128(7): 923-33; Wang et al. (2016) Arthrit. Rheumatol. 68(suppl 10):1085; incorporated herein by reference in their entirety). Daratumumab, a commercially available CD38 antibody licensed for treatment of multiple myeloma, effectively depleted plasma cells and PBs in peripheral blood mononuclear cells from patients with SLE in a dose-dependent manner in vitro (Ramaschi et al. (1996) Blood 87(6): 2308-13; incorporated herein by reference in its entirety).

AB79 (the drug substance component of mezagitamab, also known as TAK-079) is a fully human IgG1 monoclonal antibody (mAb) that binds specifically to CD38 with high affinity (Kd=3.5 nM) (U.S. Pat. No. 8,362,211, the contents of which is hereby incorporated by reference in its entirety). The available nonclinical and first-in-human data demonstrated an acceptable safety profile and encouraging pharmacodynamic (PD) effects in reducing target cells expressing CD38 (Korver et al. (2019) J. Pharmacol. Exp. Ther. 370(2): p. 182-196; Fedyk et al. (2020) Br. J. Clin. Pharmacol. 86(7): p. 1314-1325; incorporated by reference herein in their entireties). AB79 reduces the level of plasma cells and plasmablasts in blood isolated from healthy subjects and systemic lupus erythematosus (SLE) patients. In the case of SLE, 80% of the plasma cell population including short- and long-lived plasma cells, is reduced. Additionally, the number of cells producing pathogenic autoantibodies was also reduced, including VH4-34 9G4+ antibodies (70% reduction), anti-Ro antibody (70% reduction), and anti-dsDNA antibody (80% reduction).

The level of unmet need in treating patients with moderate to severe SLE remains high, and therapies with novel mechanisms of action are needed to provide improved efficacy in reducing disease activity, to help decrease use of corticosteroids, and to improve quality of life for patients with SLE.

It is an objective of the present invention to provide methods of treating patients with moderate to severe SLE by subcutaneous administration of AB79 (as the drug substance component of Mezagitamab), a fully human IgG1 mAb that binds with high affinity to CD38.

### SUMMARY OF THE INVENTION

Provided herein are methods and unit dosage forms comprising anti-CD38 antibodies or antigen binding fragments thereof used in treating patients with moderate to severe SLE.

In one aspect, the invention provides a method of treating moderate or severe SLE in a subject, the method comprising administering to the subject an isolated human anti-CD38 antibody or antigen binding fragment thereof, wherein the antibody or antigen binding fragment thereof comprises: a variable heavy chain (VH) region comprising a CDR1 having the amino acid sequence of SEQ ID NO:3, a CDR2 having the amino acid sequence of SEQ ID NO:4, and a CDR3 having the amino acid sequence of SEQ ID NO:5; and a variable light chain (VL) region comprising a CDR1 having the amino acid sequence of SEQ ID NO:6, a CDR2 having the amino acid sequence of SEQ ID NO:7, and a CDR3 having the amino acid sequence of SEQ ID NO:8; and wherein the antibody or antigen binding fragment thereof is subcutaneously administered in a dosage of from about 40 milligrams to about 140 milligrams. In some embodiments, the subject is diagnosed with severe SLE.

In another aspect, the invention provides a method of reducing the level of plasmablasts and/or plasma cells in a subject diagnosed with moderate or severe SLE, the method comprising administering to the subject an isolated human anti-CD38 antibody or antigen binding fragment thereof, wherein the antibody or antigen binding fragment thereof comprises: a VH region comprising a CDR1 having the amino acid sequence of SEQ ID NO:3, a CDR2 having the amino acid sequence of SEQ ID NO:4, and a CDR3 having the amino acid sequence of SEQ ID NO:5; and a VL region comprising a CDR1 having the amino acid sequence of SEQ ID NO:6, a CDR2 having the amino acid sequence of SEQ ID NO:7, and a CDR3 having the amino acid sequence of SEQ ID NO:8; and wherein the antibody or antigen binding fragment thereof is subcutaneously administered in a dosage of from about 40 milligrams to about 140 milligrams. In some embodiments, the subject is diagnosed with severe SLE.

In one aspect, the invention provides a method of reducing the level of immunoglobulin(s) in a subject diagnosed with moderate or severe SLE, the method comprising administering to the subject an isolated human anti-CD38 antibody or antigen binding fragment thereof, wherein the antibody or antigen binding fragment thereof comprises: a VH region comprising a CDR1 having the amino acid sequence of SEQ ID NO:3, a CDR2 having the amino acid sequence of SEQ ID NO:4, and a CDR3 having the amino acid sequence of SEQ ID NO:5; and a VL region comprising a CDR1 having the amino acid sequence of SEQ ID NO:6, a CDR2 having the amino acid sequence of SEQ ID NO:7, and a CDR3 having the amino acid sequence of SEQ ID NO:8; and wherein the antibody or antigen binding fragment thereof is subcutaneously administered in a dosage of from about 40 milligrams to about 140 milligrams. In some embodiments, the subject is diagnosed with severe SLE.

In another aspect, the invention provides the method of reducing the level of immunoglobulin(s) in a subject diagnosed with moderate or severe SLE as disclosed herein, wherein the immunoglobulin is IgA, IgG and/or IgM.

In one aspect, the invention provides a method of reducing the level of one or more autoantibodies in a subject diagnosed with moderate or severe SLE, the method comprising administering to the subject an isolated human anti-CD38 antibody or antigen binding fragment thereof, wherein the antibody or antigen binding fragment thereof comprises: (a) a VH region comprising a CDR1 having the amino acid sequence of SEQ ID NO:3, a CDR2 having the amino acid sequence of SEQ ID NO:4, and a CDR3 having the amino acid sequence of SEQ ID NO:5; and (b) a VL region comprising a CDR1 having the amino acid sequence of SEQ ID NO:6, a CDR2 having the amino acid sequence of SEQ ID NO:7, and a CDR3 having the amino acid sequence of SEQ ID NO:8; and wherein the antibody or antigen binding fragment thereof is subcutaneously administered in a dosage of from about 40 milligrams to about 140 milligrams. In some embodiments, the subject is diagnosed with severe SLE.

In another aspect, the invention provides the method of reducing the level of one or more autoantibodies in a subject diagnosed with moderate or severe SLE as disclosed herein, wherein the one or more autoantibodies is selected from the group consisting of anti-dsDNA, anti-SmDp, beta-2 glycoprotein 1 IgM, ribonucleoprotein-70, Sjogrens SS-A and Sjogrens SS-B.

In one aspect, the invention provides the methods as disclosed herein, wherein the antibody or antigen binding fragment thereof further comprises one or more engineered glycoforms, wherein the engineered glycoform comprises glycosylation of one or more polypeptides, optionally wherein the glycosylation is N-linked glycosylation or O-linked glycosylation, and optionally wherein the glycosylation is N-linked glycosylation.

In another aspect, the invention provides the methods as disclosed herein, wherein the VH region of the antibody or antigen binding fragment thereof comprises an amino acid sequence having an identity of at least 90% to SEQ ID NO:9, and/or the VL region of the antibody or antigen binding fragment thereof comprises an amino acid sequence having an identity of at least 90% to SEQ ID NO:10; optionally wherein the VH region of the antibody or antigen binding fragment thereof comprises an amino acid sequence having an identity of at least 95% to SEQ ID NO:9, and/or the VL region of the antibody or antigen binding fragment thereof comprises an amino acid sequence having an identity of at least 95% to SEQ ID NO:10; optionally wherein the VH region of the antibody or antigen binding fragment thereof comprises an amino acid sequence having an identity of at least 99% to SEQ ID NO:9, and/or the VL region of the antibody or antigen binding fragment thereof comprises an amino acid sequence having an identity of at least 99% to SEQ ID NO:10; optionally wherein the heavy chain (HC) of the antibody or antigen binding fragment thereof comprises an amino acid sequence having an identity of at least 95% to SEQ ID NO: 11; and/or the light chain (LC) of the antibody or antigen binding fragment thereof comprises an amino acid sequence having an identity of at least 95% to SEQ ID NO:12; optionally wherein the isolated antibody or antigen binding fragment thereof interacts with at least K121, F135, Q139, D141, E239, W241, C275, K276, F284, P291 and E292 of SEQ ID NO:1 and SEQ ID NO:2, based on human sequence numbering; optionally wherein the isolated antibody or antigen binding fragment thereof binds to human CD38 (SEQ ID NO:1) with a KD of 10⁻⁸ M or a greater affinity, and wherein the affinity is measured by a standard Biacore assay; optionally wherein the VH region comprises SEQ ID NO:9 and the VL region comprises SEQ ID NO:10; and optionally wherein the isolated antibody or antigen binding fragment thereof comprises an HC as set forth in SEQ ID NO:11 and an LC as set forth in SEQ ID NO:12.

In one aspect, the invention provides the methods as disclosed herein, wherein the antibody or antigen binding fragment thereof further comprises an Fc domain; optionally wherein the Fc domain is a human Fc domain or a variant Fc domain; and optionally wherein the isolated antibody or antigen binding fragment is a human IgG antibody, optionally wherein the human IgG antibody is a human IgG1 antibody.

In another aspect, the invention provides the methods as disclosed herein, wherein the subject receives background SLE medication(s), optionally wherein the background SLE medication(s) is selected from the group consisting of immunosuppressants, steroids and immunoglobulin; and optionally wherein the background SLE medication(s) is selected from the group consisting of hydroxychloroquine, hydroxychloroquine sulfate, prednisone, methylprednisolone, gabapentin, mycophenolate mofetil, and/or mycophenolic acid.

In one aspect, the invention provides the methods as disclosed herein, wherein the background SLE medication(s) is administered in combination with the antibody or antigen binding fragment thereof.

In another aspect, the invention provides the methods as disclosed herein, wherein administering the antibody or antigen binding fragment thereof results in less than 10% incidence of grade 3 or 4 of one or more treatment-related adverse events (TRAEs) or treatment-emergent adverse events (TEAEs); optionally wherein the TRAEs or TEAEs are selected from the group consisting of gastrointestinal disorders, nausea, infestations, pyrexia, Herpes Zoster, urinary tract infection, skin and cutaneous tissue disorders, headache, fever, chills/rigors, vomiting, diarrhea, arthralgia, myalgia, hypotension, respiratory, thoracic and mediastinal disorders, thrombocytopenia, leukopenia, lymphopenia, cardiac disorders, palpitations, and dyspnea; and optionally wherein administering the antibody or antigen binding fragment thereof results in one or more TRAEs or TEAEs having a maximum intensity of Common Terminology Criteria for Adverse Events (CTCAE) Grade 1 or Grade 2.

In one aspect, the invention provides the methods as disclosed herein, wherein the antibody or antigen binding fragment thereof is administered in a dosage selected from the group consisting of about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, and about 140 mg; and optionally wherein the antibody or antigen binding fragment thereof is administered in a dosage of about 45 mg, about 90 mg or about 135 mg.

In another aspect, the invention provides the methods as disclosed herein, wherein the dosage is a dosage administered once every week, once every two weeks, once every three weeks or once every four weeks.

In one aspect, the invention provides the methods as disclosed herein, wherein the antibody or antigen binding fragment thereof is administered in the form of a pharmaceutically acceptable composition, and optionally wherein the pharmaceutically acceptable composition comprises the isolated antibody or antibody fragment thereof and at least one pharmaceutically acceptable carrier, excipient or stabilizer.

In another aspect, the invention provides the methods as disclosed herein, wherein the isolated antibody or antigen binding fragment thereof comprises an HC as set forth in SEQ ID NO:11 and an LC as set forth in SEQ ID NO:12; and wherein the antibody or antigen binding fragment thereof is subcutaneously administered once every 3 weeks for 12 weeks.

In one aspect, the invention provides the methods as disclosed herein, wherein the isolated antibody or antigen binding fragment thereof is Mezagitamab.

In another aspect, the invention provides a unit dosage form comprising an isolated antibody or antigen binding fragment thereof that comprises a VH region comprising a CDR1 having the amino acid sequence of SEQ ID NO:3, a CDR2 having the amino acid sequence of SEQ ID NO:4, and a CDR3 having the amino acid sequence of SEQ ID NO:5; and a VL region comprising a CDR1 having the amino acid sequence of SEQ ID NO:6, a CDR2 having the amino acid sequence of SEQ ID NO:7, and a CDR3 having the amino acid sequence of SEQ ID NO:8; wherein the isolated antibody or antigen binding fragment thereof binds to human CD38 (SEQ ID NO:1), and the unit dosage form is formulated for subcutaneous administration of the antibody or antigen binding fragment thereof at a dosage of from 40 milligrams to 140 milligrams in the treatment of moderate or severe systemic lupus erythematosus (SLE).

In one aspect, the invention provides the unit dosage form as disclosed herein, wherein the unit dosage form is formulated for subcutaneous administration of the antibody or antigen binding fragment thereof in the treatment of severe SLE. In another aspect, the invention provides the unit dosage forms as disclosed herein, wherein the antibody or antigen binding fragment thereof further comprises one or more engineered glycoforms, wherein the engineered glycoform comprises glycosylation of one or more polypeptides, optionally wherein the glycosylation is N-linked glycosylation or O-linked glycosylation, and optionally wherein the glycosylation is N-linked glycosylation.

In one aspect, the invention provides the unit dosage forms as disclosed herein, wherein the VH region of the antibody or antigen binding fragment thereof comprises an amino acid sequence having an identity of at least 90% to SEQ ID NO:9, and/or the VL region of the antibody or antigen binding fragment thereof comprises an amino acid sequence having an identity of at least 90% to SEQ ID NO:10; optionally wherein the VH region comprises an amino acid sequence having an identity of at least 95% to SEQ ID NO:9, and/or the VL region comprises an amino acid sequence having an identity of at least 95% to SEQ ID NO:10; optionally wherein the VH region comprises an amino acid sequence having an identity of at least 99% to SEQ ID NO:9, and/or the VL region comprises an amino acid sequence having an identity of at least 99% to SEQ ID NO:10; optionally wherein the HC of the antibody or antigen binding fragment thereof comprises an amino acid sequence having an identity of at least 95% to SEQ ID NO:11, and/or the LC of the antibody or antigen binding fragment thereof comprises an amino acid sequence having an identity of at least 95% to SEQ ID NO:12; optionally wherein the isolated antibody or antigen binding fragment thereof interacts with at least K121, F135, Q139, D141, E239, W241, C275, K276, F284, P291 and E292 of SEQ ID NO:1 and SEQ ID NO:2, based on human sequence numbering; optionally wherein the isolated antibody or antigen binding fragment thereof binds to human CD38 (SEQ ID NO:1) with a KD of 10⁻⁸ M or a greater affinity, and wherein the affinity is measured by a standard Biacore assay; optionally wherein the VH region comprises SEQ ID NO:9 and the VL region comprises SEQ ID NO:10; and optionally wherein the isolated antibody or antigen binding fragment thereof comprises an HC as set forth in SEQ ID NO:11 and an LC as set forth in SEQ ID NO:12.

In another aspect, the invention provides the unit dosage forms as disclosed herein, wherein the isolated antibody or antigen binding fragment thereof further comprises an Fc domain, optionally wherein the Fc domain is a human Fc domain or a variant Fc domain; and optionally wherein the isolated antibody or antigen binding fragment is a human IgG antibody, optionally wherein the human IgG antibody is a human IgG1 antibody.

In one aspect, the invention provides the unit dosage forms as disclosed herein, wherein the isolated antibody or antigen binding fragment thereof is used in combination with one or more background SLE medications, optionally wherein the background SLE medication(s) is selected from the group consisting of immunosuppressants, steroids and immunoglobulin; optionally wherein the background SLE medication(s) is selected from the group consisting of hydroxychloroquine, hydroxychloroquine sulfate, prednisone, methylprednisolone, gabapentin, mycophenolate mofetil, and/or mycophenolic acid; and optionally wherein the unit dosage form further comprises the one or more background SLE medications.

In another aspect, the invention provides the unit dosage forms as disclosed herein, wherein administering the antibody or antigen binding fragment thereof results in less than 10% incidence of grade 3 or 4 of one or more treatment-related adverse events (TRAEs) or treatment-emergent adverse events (TEAEs); optionally wherein the TRAEs or TEAEs are selected from the group consisting of gastrointestinal disorders, nausea, infestations, pyrexia, Herpes Zoster, urinary tract infection, skin and cutaneous tissue disorders, headache, fever, chills/rigors, vomiting, diarrhea, arthralgia, myalgia, hypotension, respiratory, thoracic and mediastinal disorders, thrombocytopenia, leukopenia, lymphopenia, cardiac disorders, palpitations, and dyspnea; and optionally wherein administering the antibody or antigen binding fragment thereof results in one or more TRAEs or TEAEs having a maximum intensity of Common Terminology Criteria for Adverse Events (CTCAE) Grade 1 or Grade 2.

In one aspect, the invention provides the unit dosage forms as disclosed herein, wherein the antibody or antigen binding fragment thereof is administered in a dosage selected from the group consisting of about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, and about 140 mg; and optionally wherein the antibody or antigen binding fragment thereof is administered in a dosage of about 45 mg, about 90 mg or about 135 mg.

In another aspect, the invention provides the unit dosage forms as disclosed herein, wherein the dosage is a dosage administered once every week, once every two weeks, once every three weeks or once every four weeks.

In one aspect, the invention provides the unit dosage forms as disclosed herein further comprising at least one pharmaceutically acceptable carrier, excipient, or stabilizer.

In another aspect, the invention provides the unit dosage forms as disclosed herein, wherein the isolated antibody or antigen binding fragment thereof comprises an HC as set forth in SEQ ID NO:11 and an LC as set forth in SEQ ID NO:12; and wherein the antibody or antigen binding fragment thereof is subcutaneously administered once every 3 weeks for 12 weeks.

In a further aspect, the invention provides the unit dosage forms as disclosed herein, wherein the isolated antibody or antigen binding fragment thereof is Mezagitamab.

In one aspect, the invention provides a human anti-CD38 antibody or antigen binding fragment thereof for use in treating moderate or severe systemic lupus erythematosus (SLE), reducing the level of plasmablasts and/or plasma cells in a subject diagnosed with moderate or severe SLE, reducing the level of immunoglobulin(s) in a subject diagnosed with moderate or severe SLE, or reducing the level of one or more autoantibodies in a subject diagnosed with moderate or severe SLE, wherein the antibody or antigen binding fragment thereof comprises: a VH region comprising a CDR1 having the amino acid sequence of SEQ ID NO:3, a CDR2 having the amino acid sequence of SEQ ID NO:4, and a CDR3 having the amino acid sequence of SEQ ID NO:5; and a VL region comprising a CDR1 having the amino acid sequence of SEQ ID NO:6, a CDR2 having the amino acid sequence of SEQ ID NO:7, and a CDR3 having the amino acid sequence of SEQ ID NO:8; and wherein the antibody or antigen binding fragment thereof is subcutaneously administered in a dosage of from about 40 milligrams to about 140 milligrams.

In one aspect, the invention provides a human anti-CD38 antibody or antigen binding fragment thereof for use in treating moderate or severe systemic lupus erythematosus (SLE), wherein the antibody or antigen binding fragment thereof comprises: a VH region comprising a CDR1 having the amino acid sequence of SEQ ID NO:3, a CDR2 having the amino acid sequence of SEQ ID NO:4, and a CDR3 having the amino acid sequence of SEQ ID NO:5; and a VL region comprising a CDR1 having the amino acid sequence of SEQ ID NO:6, a CDR2 having the amino acid sequence of SEQ ID NO:7, and a CDR3 having the amino acid sequence of SEQ ID NO:8; and wherein the antibody or antigen binding fragment thereof is subcutaneously administered in a dosage of from about 40 milligrams to about 140 milligrams.

In one aspect, the invention provides a human anti-CD38 antibody or antigen binding fragment thereof for use in reducing the level of plasmablasts and/or plasma cells in a subject diagnosed with moderate or severe SLE, wherein the antibody or antigen binding fragment thereof comprises: a VH region comprising a CDR1 having the amino acid sequence of SEQ ID NO:3, a CDR2 having the amino acid sequence of SEQ ID NO:4, and a CDR3 having the amino acid sequence of SEQ ID NO:5; and a VL region comprising a CDR1 having the amino acid sequence of SEQ ID NO:6, a CDR2 having the amino acid sequence of SEQ ID NO:7, and a CDR3 having the amino acid sequence of SEQ ID NO:8; and wherein the antibody or antigen binding fragment thereof is subcutaneously administered in a dosage of from about 40 milligrams to about 140 milligrams.

In one aspect, the invention provides a human anti-CD38 antibody or antigen binding fragment thereof for use in reducing the level of immunoglobulin(s) in a subject diagnosed with moderate or severe SLE, wherein the antibody or antigen binding fragment thereof comprises: a VH region comprising a CDR1 having the amino acid sequence of SEQ ID NO:3, a CDR2 having the amino acid sequence of SEQ ID NO:4, and a CDR3 having the amino acid sequence of SEQ ID NO:5; and a VL region comprising a CDR1 having the amino acid sequence of SEQ ID NO:6, a CDR2 having the amino acid sequence of SEQ ID NO:7, and a CDR3 having the amino acid sequence of SEQ ID NO:8; and wherein the antibody or antigen binding fragment thereof is subcutaneously administered in a dosage of from about 40 milligrams to about 140 milligrams. In some embodiments, the immunoglobulin is IgA, IgG and/or IgM.

In one aspect, the invention provides a human anti-CD38 antibody or antigen binding fragment thereof for use in reducing the level of one or more autoantibodies in a subject diagnosed with moderate or severe SLE, wherein the antibody or antigen binding fragment thereof comprises: a VH region comprising a CDR1 having the amino acid sequence of SEQ ID NO:3, a CDR2 having the amino acid sequence of SEQ ID NO:4, and a CDR3 having the amino acid sequence of SEQ ID NO:5; and a VL region comprising a CDR1 having the amino acid sequence of SEQ ID NO:6, a CDR2 having the amino acid sequence of SEQ ID NO:7, and a CDR3 having the amino acid sequence of SEQ ID NO:8; and wherein the antibody or antigen binding fragment thereof is subcutaneously administered in a dosage of from about 40 milligrams to about 140 milligrams. In some embodiments, the one or more autoantibodies is selected from the group consisting of anti-dsDNA, anti-SmDp, beta-2 glycoprotein 1 IgM, ribonucleoprotein-70, Sjogrens SS-A and Sjogrens SS-B.

In one aspect, the invention provides a human anti-CD38 antibody or antigen binding fragment thereof for use in treating moderate or severe systemic lupus erythematosus (SLE) in a subject, wherein (i) the level of plasmablasts and/or plasma cells are reduced in the subject; (ii) the level of immunoglobulin(s) are reduced in the subject; and/or (iii) the level of one or more autoantibodies are reduced in the subject; wherein the antibody or antigen binding fragment thereof comprises: a VH region comprising a CDR1 having the amino acid sequence of SEQ ID NO:3, a CDR2 having the amino acid sequence of SEQ ID NO:4, and a CDR3 having the amino acid sequence of SEQ ID NO:5; and a VL region comprising a CDR1 having the amino acid sequence of SEQ ID NO:6, a CDR2 having the amino acid sequence of SEQ ID NO:7, and a CDR3 having the amino acid sequence of SEQ ID NO:8; and wherein the antibody or antigen binding fragment thereof is subcutaneously administered in a dosage of from about 40 milligrams to about 140 milligrams. In some embodiments, the immunoglobulin is IgA, IgG and/or IgM. In some embodiments, the one or more autoantibodies is selected from the group consisting of anti-dsDNA, anti-SmDp, beta-2 glycoprotein 1 IgM, ribonucleoprotein-70, Sjogrens SS-A and Sjogrens SS-B.

### BRIEF DESCRIPTION OF THE DRAWINGS

The objects and features of the invention may be better understood by reference to the drawings described below.

FIG. 1 shows TAK-079-2001 trial design schematic. This Phase 1b study compared active mezagitamab with matching placebo in combination with a background SLE standard treatment across 3 sequentially enrolling cohorts in a double-blinded study design which included a 28-day screening period; 12-week treatment period with single SC dose injection administer every 3 weeks (total of 4 doses); a 12-week safety follow-up (FU) period; and a 12-week long term safety follow-up period.

FIG. 2 shows TAK-079-2001 study cohort reviews. This Phase 1b study compared active mezagitamab with matching placebo in combination with a background SLE standard treatment across 3 sequentially enrolling cohorts in a double-blinded study design, with safety review at the end of each cohort and 3:1 randomization to mezagitamab:placebo.

FIG. 3A-3B shows the pharmacokinetics (PK) of mezagitamab in SLE patients demonstrated nonlinear increase in exposure in the tested dose range. FIG. 3A shows mean (±SD) serum TAK-079 concentration−time profiles by treatment with dosing intervals 1-4 side by side (Pharmacokinetic Analysis Set). Vertical line at each nominal time point represents ±SD. The lower SD bar is not presented in the semi-logarithmic scale when mean−SD≤0 on the original scale. FIG. 3B shows mean (+SD) serum TAK-079 concentration−time profiles by treatment with dosing intervals 1 and 2 side by side (Pharmacokinetic Analysis Set). Vertical line at each nominal time point represents ±SD. The lower SD bar is not presented in the semi-logarithmic scale when mean−SD≤0 on the original scale. Unscheduled visits are not included.

FIG. 4A-4D shows pharmacodynamics of mezagitamab in SLE patients. FIG. 4A shows receptor occupancy and population change in NK cells. FIG. 4B shows receptor occupancy in plasmablasts (PB). FIG. 4C shows dose-dependent, longitudinal alterations in serum IgG levels, expressed as mean percent change (Pharmacodynamic Analysis Set) of pretreatment baseline ±SD. FIG. 4D shows dose-dependent, longitudinal alterations in serum IgG levels, expressed as mean % change of pretreatment baseline ±S.E.M. Baseline is treated as study day 1. * Day 85 is the end-of-dosing visit.

FIG. 5 shows percent change in serum immunoglobulin A (Pharmacodynamic Analysis Set). Vertical line at each nominal time point represents SD. Baseline is treated as study day 1. * Day 85 is the end-of-dosing visit.

FIG. 6A-6B shows temporal profiles of IgG, autoantibodies, and clinical scores. Decreases in serum IgG and autoantibodies were modest and did not appear to be correlated with each other or with clinical response. FIG. 6A shows data from subjects in the highest tested dose of 135 mg. FIG. 6B shows data from CLASI responders in mezagitamab-treated groups.

FIG. 7A-7C shows mezagitamab depleted populations in a roughly CD38-dependent fashion. FIG. 7A shows depletion of selected CD38 positive cell populations (labeled) plotted against baseline CD38 expression. FIG. 7B shows 20-cluster TSNE cluster mapping identified following CyTOF analysis reveals discrete populations (described in FIG. 8A and FIG. 8B) that are maintained over the course of study. FIG. 7C shows CD38 heatmap expression throughout study.

FIG. 8A shows twenty cluster analysis with phenotypic heatmap of PBMCs from mezagitamab-treated patients. FIG. 8B shows a labeled TSNE plot.

FIG. 9A-9C shows fifty cluster analysis of PBMCs from mezagitamab-treated patients. FIG. 9A shows phenotypic heat map and cluster size (as % of selected events). Treatment effect evaluation identified two clusters with phenotypes consistent with effector-like CD8 T cells (cluster 32, FIG. 9B) and CD4 T cells (cluster 34, FIG. 9C). p-value<0.05 (*), 0.01 (**), or 0.001 (***) of 135 mg dose when compared to placebo.

FIG. 10 shows pharmacokinetics of mezagitamab in SLE patients demonstrated nonlinear increase in exposure in the tested dose range.

FIG. 11 shows LS mean change from baseline of CLASI total activity score by treatment group—MMRM (Safety Analysis Set). CLASI: Cutaneous Lupus Erythematosus Disease Area and Severity Index; MMRM: mixed-effect model for repeated measures. From a MMRM analysis over all postbaseline visits, with the change from baseline as the outcome, treatment, visit, and (treatment×visit) interaction terms as the factors, and baseline value and (baseline×visit) interaction as the covariates. Kenward-Rogers method is used to calculate the degrees of freedom and covariance matrix is based on autoregressive (1) covariance structure. The vertical line at each study day represents ±SEM. * Day 85 is the end-of-dosing visit.

FIG. 12 shows individual CLASI observed values by Baseline Total Activity Score Category (Safety Analysis Set). CLASI responders demonstrated substantial, clinically meaningful improvements in cutaneous manifestations by the end of treatment. Subgroup analysis for total activity scores revealed that all subjects with a baseline CLASI score of >10 met responder criteria at end of treatment. CLASI: cutaneous lupus erythematosus disease area and severity index. Baseline is treated as study day 1.

FIG. 13 shows LS mean change from baseline of SLEDAI-2K Total Score by Treatment Group—MMRM (Safety Analysis Set). MMRM: mixed-effect model for repeated measures; SLEDAI-2K: Systemic Lupus Erythematosus Disease Activity Index—2000. From a MMRM analysis over all postbaseline visits, with the change from baseline as the outcome, treatment, visit, and (treatment×visit) interaction terms as the factors, and baseline value and (baseline×visit) interaction as the covariates. Kenward-Rogers method is used to calculate the degrees of freedom and covariance matrix is based on autoregressive (1) covariance structure. The vertical line at each study day represents ±SEM. * Day 85 is the end-of-dosing visit.

FIG. 14 shows individual SLEDAI-2K observed values by Baseline Total Score Category (Safety Analysis Set). SLEDAI-2K: Systemic Lupus Erythematosus Disease Activity Index—2000. Baseline is treated as study day 1.

### DETAILED DESCRIPTION OF THE INVENTION

The present invention relates to methods and unit dosage forms for subcutaneous administration of a therapeutically effective amount of an isolated anti-CD38 antibody to treat patients with moderate or severe Systemic Lupus Erythematosus (SLE).

SLE is a serious condition that is characterized by aberrant activity of the immune system, leading to variable clinical symptoms. Treating and identifying novel therapies for SLE is challenging because of its genetic and phenotypic heterogeneity, leaving a significant unmet medical need for patients especially with moderate to severe SLE (Kiriakidou and Ching (2020) Ann. Intern. Med. 172(11): ITC81-ITC96, incorporated herein by reference in its entirety).

SLE is characterized by dysregulation of T and B lineage cells, as well as other components of the innate immune system. A hallmark of the disease is the production of pathogenic autoantibodies to double-stranded DNA (dsDNA), phospholipids, blood cells, and other targets. Tissue damage in SLE is caused primarily by these pathogenic autoantibodies through immune complex deposition with Fc- and complement-mediated inflammation, as well as through direct antibody-target interactions (Rahman and Isenberg (2008) N. Engl. J. Med. 358(9): 929-39; incorporated herein by reference in its entirety). Virtually any organ or system in the body can be affected by SLE (D'Cruz (2006) Blood (ASH Annual Meeting Abstracts) 129: 2359-67; Lateef and Petri (2012) Arthritis Res. Ther. 14(Suppl 4): S4; incorporated herein by reference in their entirety).

The clinical course of SLE is episodic, with flares of disease activity that lead to increased disability and organ damage over time. For moderate to severe cases, the current standard of care includes off-label therapies, including immunosuppressants (azathioprine, cyclophosphamide, and mycophenolate mofetil), high-dose steroids, and intravenous (IV) immunoglobulin for cytopenia. The therapeutic agents that are used for treatment of SLE have demonstrated limited success. Short-lived plasmablasts (PBs), plasma cells (i.e., terminally differentiated B cells), and long-lived plasma cells produce the characteristic pathogenic autoantibodies and are, therefore, critically involved in SLE pathogenesis.

Studies have shown an increased number of plasmablasts (PBs) in the blood of patients with active SLE (D'Cruz (2006) Blood (ASH Annual Meeting Abstracts) 129: 2359-67; Clemens et al. (2017) Clin. Pharmacokinet. 56(8): 915-24; Sanada et al. (2016) Blood 128(7): 923-33; Wang et al. (2016) Arthrit. Rheumatol. 68(suppl 10): 1085; incorporated by reference in their entirety). CD38 is a type II glycoprotein that is highly and uniformly expressed on antibody-producing PBs and plasma cells (Sullivan et al. (2017) Blood 129(22): 3033-7; incorporated herein by reference in its entirety), making it a potential target for treatment of SLE. In an ex vivo study of CD38 expression on various immune cells in peripheral blood mononuclear cells from patients with SLE, the highest CD38 expression was observed on plasma cells and PBs, followed by natural killer (NK) cells, plasmacytoid dendritic cells, a regulatory T cell subpopulation, and naïve T cells (Ramaschi et al. (1996) Blood 87(6): 2308-13; incorporated herein by reference in its entirety).

The significantly higher CD38 expression on plasma cells and PBs compared with other immune cells suggests the potential for selectively depleting these cells with an anti-CD38 antibody. Daratumumab, a commercially available anti-CD38 antibody, effectively depleted plasma cells and PBs in peripheral blood mononuclear cells from patients with SLE in a dose-dependent manner in vitro (Ramaschi et al. (1996) Blood 87(6): 2308-13; incorporated herein by reference in its entirety). Intravenous daratumumab has been approved for patients with multiple myeloma (relapsed and newly diagnosed). However, the most frequent adverse reactions (≥20%) with daratumumab monotherapy or in combination with standard anti-myeloma regimens are infusion-related reactions (IRRs), neutropenia, thrombocytopenia, fatigue, nausea, diarrhea, constipation, vomiting, muscle spasms, arthralgia, back pain, pyrexia, chills, dizziness, insomnia, cough, dyspnea, peripheral edema, peripheral sensory neuropathy, and upper respiratory tract infections (Darzalex USPI). Daratumumab can cause severe and/or serious infusion reactions including anaphylactic reactions and have been reported in approximately 30 half of all patients (Darzalex USPI). Attention must also be paid to daratumumab interference with certain laboratory assays, which importantly may complicate blood compatibility testing. (Darzalex USPI).

Other Morphosys antibodies targeting CD38 are known (see, e.g., WO 2006/125640 incorporated herein by reference in its entirety, which discloses four human antibodies: MOR03077, MOR03079, MOR03080, and MOR03100 and two murine antibodies: OKT10 and 1B4). These prior art antibodies are inferior to TAK-079, another anti-CD38 antibody, for a variety of reasons. MOR03080 binds to human CD38 and cynomolgus CD38 but with a low affinity to human CD38 (Biacore KD=27.5 nm). OKT10 binds to human CD38 and cynomolgus CD38 but with a low/moderate affinity to human CD38 (Biacore KD=8.28 nm). MOR03079 binds to human CD38 with a high affinity (Biacore KD=2.4 nm) but does not bind to cynomolgus CD38. MOR03100 and MOR03077 bind to human CD38 with moderate or low affinity (Biacore KD=10 nm and 56 nm, respectively). By comparison, TAK-079 binds to human and cynomolgus CD38 with a high affinity (to human CD38 with Biacore KD=5.4 nm). Moreover, the prior art antibodies have poor ADCC as well as CDC activity.

An advantage of more efficient ADCC is the ability to deliver an anti-CD38 therapeutic as a low volume injection. A safety profile and PD target effect was observed after TAK-079, at a dose up to 0.6 mg/kg dose was subcutaneously administered to healthy subjects. A single subcutaneous dose of 0.6 mg/kg TAK-079 reduced the level of PBs in peripheral blood >90% and NK cells >80% without comparable reductions in monocytes and B and T cells. Levels of PBs and NK cells recovered to 50% of baseline levels 21 days after administration, on average. At this dose, there were no Serious Adverse Events (SAEs), on-study deaths, or Adverse Events (AEs) that led to study discontinuation (WO 2019/140410, incorporated herein by reference in its entirety). Further studies showed that after TAK-079 was subcutaneously administered at a dosage of 45 mg, 135 mg, 300 mg, or 600 mg to patients with relapsed and/or refractory multiple myeloma (RRMM), no drug-related SAEs, on-study deaths, or AEs that led to study discontinuation were reported. Administration of TAK-079 reduced levels of plasmablasts in blood and bone marrow aspirates as well as plasma cells in bone marrow aspirates in a dose dependent manner. In patients with advanced RRMM, TAK-079 also showed early signs of anti-tumor activity as evidenced by at least 50% reduction in disease burden in some patients and prolonged disease stabilization in others (WO 2019/186273; incorporated herein by reference in its entirety). However, the feasibility and efficacy of administering TAK-079 in treating patients with SLE, especially those with moderate or severe SLE is unknown.

The methods and unit dosages of the present disclosure provide, for the first time, subcutaneous administration of therapeutically effective dosages of anti-CD38 antibodies in treating patients with moderate or severe SLE.

The present invention provides methods and unit dosage forms for subcutaneous administration of a therapeutically effective amount of an isolated anti-CD38 antibody or antigen binding fragment to a patient with moderate or severe SLE. In some embodiments, the antibody or antigen binding fragment for subcutaneous administration comprises a variable heavy chain (VH) region comprising or consisting of SEQ ID NO:9 (or a sequence with at least 80%, 85%, 90%, 95%, 97% or 99% sequence identity thereto) and a variable light chain (VL) region comprising or consisting of SEQ ID NO:10 (or a sequence with at least 80%, 85%, 90%, 95%, 97% or 99% sequence identity thereto). In some embodiments, the patient is diagnosed with moderate SLE. In some embodiments, the patient is diagnosed with severe SLE. In some embodiment, the antibody or antigen binding fragment thereof is subcutaneously administered in a dosage of from 40 milligrams to 140 milligrams.

Unless otherwise defined herein, scientific and technical terms used in connection with the present invention shall have the meanings that are commonly understood by those of ordinary skill in the art. The meaning and scope of the terms should be clear. However, in the event of any latent ambiguity, definitions provided herein take precedence over any dictionary or extrinsic definition. Further, unless otherwise required by context, singular terms shall include pluralities and plural terms shall include the singular. The term “or” includes “and/or” unless stated otherwise. Furthermore, the use of the term “including,” “includes,” or “included” is not limiting. Terms such as “element” and “component” encompass both elements and components comprising one unit and elements and components that comprise more than one subunit unless specifically stated otherwise.

The methods and techniques of the present invention are generally performed according to conventional methods well known in the art and as described in various general and more specific references that are cited and discussed throughout the present specification unless otherwise indicated. Standard techniques are used for chemical syntheses, chemical analyses, pharmaceutical preparation, formulation, delivery, and treatment of patients. Commercial enzymatic reactions and purification techniques are performed according to manufacturer's specifications, as commonly accomplished in the art or as described herein.

All headings and section designations are used for clarity and reference purposes only and are not to be considered limiting in any way. For example, those of skill in the art will appreciate the usefulness of combining various aspects of the disclosure from different headings and sections as appropriate according to the spirit and scope of the invention described herein.

Select terms are defined below in order for the present invention to be more readily understood.

The terms “human CD38” and “human CD38 antigen” refer to the amino acid sequence of SEQ ID NO:1, or a functional fraction thereof, such as an epitope, as defined herein (Table 1). In general, CD38 possesses a short intracytoplasmic tail, a transmembrane domain, and an extracellular domain. The terms “cynomolgus CD38” and “cynomolgus CD38 antigen” refer to the amino acid sequence of SEQ ID NO:2, which is 92% identical to the amino acid sequence of human CD38 (Table 1). Synonyms for CD38 include cyclic ADP ribose hydrolase; cyclic ADP ribose-hydrolase 1; ADP ribosyl cyclase; ADP-ribosyl cyclase 1; cADPr hydrolase 1; CD38-rs1; I-19; NIM-R5 antigen; 2′-phospho-cyclic-ADP-ribose transferase; 2′-phospho-ADP-ribosyl cyclase; 2′-phospho-cyclic-ADP-ribose transferase; 2′-phospho-ADP-ribosyl cyclase; and T10.

TABLE 1
Amino Acid Sequence of Human and Cynomolgus Monkey CD38

| | Amino Acid Sequence | SEQ |
|---|---|---|
| Species | 12345678901234567890123456789012345678901234567890 | ID NO |
| Human | MANCEFSPVSGDKPCCRLSRRAQLCLGVSILVLILVVVL | 1 |
| CD38 | AVVVPRWRQQWSGPGTTKRFPETVLARCVKYTEIHPE | |
| | MRHVDCQSVWDAFKGAFISKHPCNITEEDYQPLMKLG | |
| | TQTVPCNKILLWSRIKDLAHQFTQVQRDMFTLEDTLLG | |
| | YLADDLTWCGEFNTSKINYQSCPDWRKDCSNNPVSVF | |
| | WKTVSRRFAEAACDVVHVMLNGSRSKIFDKNSTFGSVE | |
| | VHNLQPEKVQTLEAWVIHGGREDSRDLCQDPTIKELESI | |
| | ISKRNIQFSCKNIYRPDKFLQCVKNPEDSSCTSEI | |
| Cyno | MANCEFSPVSGDKPCCRLSRRAQVCLGVCLLVLLILVV | 2 |
| CD38 | VVAVVLPRWRQQWSGSGTTSRFPETVLARCVKYTEVH | |
| | PEMRHVDCQSVWDAFKGAFISKYPCNITEEDYQPLVKL | |
| | GTQTVPCNKTLLWSRIKDLAHQFTQVQRDMFTLEDML | |
| | LGYLADDLTWCGEFNTFEINYQSCPDWRKDCSNNPVSV | |
| | FWKTVSRRFAETACGVVHVMLNGSRSKIFDKNSTFGSV | |
| | EVHNLQPEKVQALEAWVIHGGREDSRDLCQDPTIKELE | |
| | SIISKRNIRFFCKNIYRPDKFLQCVKNPEDSSCLSGI | |
| Human | MAAQGCAASRLLQLLLQLLLLLLLLAAGGARARWRGE | 13 |
| CD157 | GTSAHLRDIFLGRCAEYRALLSPEQRNKNCTAIWEAFK | |
| | VALDKDPCSVLPSDYDLFINLSRHSIPRDKSLFWENSHL | |
| | LVNSFADNTRRFMPLSDVLYGRVADFLSWCRQKNDSG | |
| | LDYQSCPTSEDCENNPVDSFWKRASIQYSKDSSGVIHV | |
| | MLNGSEPTGAYPIKGFFADYEIPNLQKEKITRIEIWVMH | |
| | EIGGPNVESCGEGSMKVLEKRLKDMGFQYSCINDYRPV | |
| | KLLQCVDHSTHPDCALKSAAAATQRKAPSLYTEQRAG | |
| | LIIPLFLVLASRTQL | |

The terms “therapeutically effective amount” and “therapeutically effective dosage” refer to an amount of a therapy that is sufficient to reduce or ameliorate the severity and/or duration of a disorder or one or more symptoms thereof, prevent the advancement of a disorder; cause regression of a disorder; prevent the recurrence, development, onset, or progression of one or more symptoms associated with a disorder; or enhance or improve the prophylactic or therapeutic effect(s) of another therapy (e.g., prophylactic or therapeutic agent), at dosages and for periods of time necessary to achieve a desired therapeutic result. A therapeutically effective amount may vary according to factors such as the disease state, age, sex, and weight of the individual, and the ability of the medicaments to elicit a desired response in the individual. A therapeutically effective amount of an antibody or antigen binding fragment thereof is one in which any toxic or detrimental effects of the antibody or antigen binding fragment thereof are outweighed by the therapeutically beneficial effects.

The terms “patient” and “subject” include both humans and other animals. Thus, the compositions, dosages, and methods disclosed herein are applicable to both human and veterinary therapies. In one embodiment, the patient is a mammal, for example, a human.

The term “isolated antibody” refers to an antibody that is substantially free of other antibodies having different antigenic specificities. For instance, an isolated antibody that specifically binds to CD38 is substantially free of antibodies that specifically bind antigens other than CD38. An isolated antibody that specifically binds to an epitope, isoform or variant of human CD38 or cynomolgus CD38 may, however, have cross-reactivity to other related antigens, for instance from other species, such as CD38 species homologs. Moreover, an isolated antibody may be substantially free of other cellular material and/or chemicals.

The term “about” refers to an extent near in number, degree, volume, time, etc., with only minor variations in dimension of up to 10%.

The term “pharmaceutically acceptable carrier” refers to a pharmaceutically acceptable material, composition or vehicle, suitable for administering compounds of the present invention to mammals. The carriers include liquid or solid filler, diluent, excipient, solvent or encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. In one embodiment, the pharmaceutically acceptable carrier is suitable for subcutaneous administration.

The term “pharmaceutical composition” refers to preparations suitable for administration to a subject and treatment of disease. When the anti-CD38 antibodies of the present invention are administered as pharmaceuticals to mammals, e.g., humans, they can be administered “as is” or as a pharmaceutical composition containing the anti-CD38 antibody in combination with a pharmaceutically acceptable carrier, excipient, and/or stabilizer. The pharmaceutical composition can be in the form of a unit dosage form for administration of a particular dosage of the anti-CD38 antibody at a particular concentration, a particular amount, or a particular volume. Pharmaceutical compositions comprising the anti-CD38 antibodies, either alone or in combination with prophylactic agents, therapeutic agents, and/or pharmaceutically acceptable carriers are provided. Suitably, the pharmaceutical composition may comprise a unit dosage form according to the present invention either alone or in combination with prophylactic agents, therapeutic agents, and/or pharmaceutically acceptable carriers. Suitably, the pharmaceutical composition may comprise a human anti-CD38 antibody as described herein either alone or in combination with prophylactic agents, therapeutic agents, and/or pharmaceutically acceptable carriers.

Traditional antibody structural units typically comprise a tetramer. Each tetramer is typically composed of two identical pairs of polypeptide chains, each pair having one “light” chain (typically having a molecular weight of about 25 kDa) and one “heavy” chain (typically having a molecular weight of about 50-70 kDa). Human light chains (LC) are classified as kappa and lambda light chains. Heavy chains (HC) are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Thus, “isotype” refers to any of the subclasses of immunoglobulins defined by the chemical and antigenic characteristics of their constant regions. The known human immunoglobulin isotypes are IgG1, IgG2, IgG3, IgG4, IgAQ1, IgA2, IgM1, IgM2, IgD, and IgE. Therapeutic antibodies can also comprise hybrids of isotypes and/or subclasses.

Each VH and VL region (about 100 to 110 amino acids in length) is composed of three hypervariable regions called “complementarity determining regions” (CDRs) and four framework regions (FRs) (about 15-30 amino acids in length), arranged from amino-terminus to carboxy-terminus in the following order: FR1-CDR1-FR2-CDR2-FR3-CDR3-FR4. “Variable” refers to the fact that the CDRs differ extensively in sequence among antibodies and thereby determines a unique antigen binding site.

The hypervariable region generally encompasses amino acid residues from about amino acid residues 24-34 (LCDR1; “L” denotes light chain), 50-56 (LCDR2) and 89-97 (LCDR3) in the VL region and around about 31-35B (HCDR1; “H” denotes heavy chain), 50-65 (HCDR2), and 95-102 (HCDR3) in the VL region (Kabat et al. (1991) Sequences Of Proteins Of Immunological Interest, 5th Ed. Public Health Service, National Institutes of Health, Bethesda, MD; incorporated herein by reference in its entirety) and/or those residues forming a hypervariable loop (e.g., residues 26-32 (LCDR1), 50-52 (LCDR2) and 91-96 (LCDR3) in the VL region and 26-32 (HCDR1), 53-55 (HCDR2) and 96-101 (HCDR3) in the VH region (Chothia and Lesk (1987) J. Mol. Biol. 196: 901-917; incorporated herein by reference in its entirety)

The Kabat numbering system is generally used when referring to a residue in the variable domain (approximately, residues 1-107 of the VL region and residues 1-113 of the VH region) (e.g., Kabat et al. (1991) Sequences Of Proteins Of Immunological Interest, 5th Ed. Public Health Service, National Institutes of Health, Bethesda, MD; incorporated herein by reference in its entirety), with the EU number system used for the Fc region.

The term “immunoglobulin (Ig) domain” refers to a region of an immunoglobulin having a distinct tertiary structure. Ig domains include VH and VL regions, CDRs, framework regions, constant region domains, and hinge regions. Each HC and LC has constant region domains referred to as constant heavy (CH) domains and constant light (CL) domains. In the context of IgG antibodies, the IgG isotypes each have a constant region comprising three CH domains. The carboxy-terminal portion of each HC and LC defines a constant region primarily responsible for effector function. Accordingly, “CH” domains in the context of IgG are as follows: “CH1” refers to positions 118-220 according to the EU index as in Kabat. “CH2” refers to positions 237-340 according to the EU index as in Kabat, and “CH3” refers to positions 341-447 according to the EU index as in Kabat.

Another type of Ig domain of the HC is the hinge region. The term “hinge region” refers to the flexible polypeptide comprising the amino acids between the first and second constant domains of an antibody. Structurally, the IgG CH1 domain ends at EU position 220, and the IgG CH2 domain begins at residue EU position 237. Thus, for IgG the antibody hinge is herein defined to include positions 221 (D221 in IgG1) to 236 (G236 in IgG1), wherein the numbering is according to the EU index as in Kabat. In some embodiments, for example in the context of an Fc region, the lower hinge is included, with the “lower hinge” generally referring to positions 226 or 230.

The term “Fc region” refers to the polypeptide comprising the constant region of an antibody excluding the CH1 domain and in some cases, part of the hinge. Thus, Fc refers to the last two constant region Ig domains (CH2 and CH3) of IgA, IgD, and IgG, the last three constant region Ig domains of IgE and IgM, and the flexible hinge N-terminal to these domains. For IgA and IgM, Fc may include the J chain. For IgG, the Fc domain comprises Ig domains Cγ2 and Cγ3 (C72 and Cγ3) and the lower hinge region between Cγ1 (Cγ1) and Cγ2 (Cγ2). Although the boundaries of the Fc region may vary, the human IgG HC Fc region is usually defined to include residues C226 or P230 to its carboxyl-terminus, wherein the numbering is according to the EU index as in Kabat. In some embodiments, as is more fully described below, amino acid modifications are made to the Fc region, for example to alter binding to one or more FcγR receptors or to the FcRn receptor.

### CD38 Antibodies

Accordingly, the present invention provides isolated anti-CD38 antibodies that specifically bind human and primate CD38 protein that find use in subcutaneous administration methods and unit dosage forms in treating patients with moderate or severe SLE. The antibodies or antigen binding fragments thereof used in the present invention bind to both the human and primate CD38 proteins, particularly primates used in clinical testing, such as cynomolgus monkeys (Macaca fascicularis, Crab eating macaque, also referred to herein as “cyno”).

“TAK-079” or “Mezagitamab” is a therapeutic protein comprising a fully human immunoglobulin IgG1 monoclonal antibody that binds specifically to CD38 with high affinity (Kd=3.5 nM) referred to herein as AB79 (U.S. Pat. No. 8,362,211, the contents of which is hereby incorporated by reference in its entirety). The amino acid sequences of Mezagitamab are shown in Table 2.

TABLE 2
Amino Acid Sequences of Mezagitamab

| | Sequence | SEQ |
|---|---|---|
| | 12345678901234567890123456789012345678901234567890 | ID NO |
| HCDR1 | GFTFDDYG | 3 |
| HCDR2 | ISWNGGKT | 4 |
| HCDR3 | ARGSLFHDSSGFYFGH | 5 |
| LCDR1 | SSNIGDNY | 6 |
| LCDR2 | RDS | 7 |
| LCDR3 | QSYDSSLSGS | 8 |
| VH | EVQLLESGGGLVQPGGSLRLSCAASGFTFDDYGMSWVRQAPGKGLEWVSD | 9 |
| | ISWNGGKTHYVDSVKGQFTISRDNSKNTLYLQMNSLRAEDTAVYYCARGS | |
| | LFHDSSGFYFGHWGQGTLVTVSSASTKGPSVFPLA | |
| VL | QSVLTQPPSASGTPGQRVTISCSGSSSNIGDNYVSWYQQLPGTAPKLLIY | 10 |
| | RDSQRPSGVPDRFSGSKSGTSASLAISGLRSEDEADYYCQSYDSSLSGSV | |
| | FGGGTKLTVLGQPKANPTVTLFPPSSEEL | |
| HC | EVQLLESGGGLVQPGGSLRLSCAASGFTFDDYGMSWVRQAPGKGLEWVSD | 11 |
| | ISWNGGKTHYVDSVKGQFTISRDNSKNTLYLQMNSLRAEDTAVYYCARGS | |
| | LFHDSSGFYFGHWGQGTLVTVSSASTKGPSVFPLAPSSKSTSGGTAALGC | |
| | LVKDYFPEPVTVSWNSGALTSGVHTFPAVLQSSGLYSLSSVVTVPSSSLG | |
| | TQTYICNVNHKPSNTKVDKRVEPKSCDKTHTCPPCPAPELLGGPSVFLFP | |
| | PKPKDTLMISRTPEVTCVVVDVSHEDPEVKFNWYVDGVEVHNAKTKPREE | |
| | QYNSTYRVVSVLTVLHQDWLNGKEYKCKVSNKALPAPIEKTISKAKGQPR | |
| | EPQVYTLPPSREEMTKNQVSLTCLVKGFYPSDIAVEWESNGQPENNYKTT | |
| | PPVLDSDGSFFLYSKLTVDKSRWQQGNVFSCSVMHEALHNHYTOKSLSLS | |
| | PGK | |
| LC | QSVLTQPPSASGTPGQRVTISCSGSSSNIGDNYVSWYQQLPGTAPKLLIY | 12 |
| | RDSQRPSGVPDRFSGSKSGTSASLAISGLRSEDEADYYCQSYDSSLSGSV | |
| | FGGGTKLTVLGQPKANPTVTLFPPSSEELQANKATLVCLISDFYPGAVTV | |
| | AWKADGSPVKAGVETTKPSKQSNNKYAASSYLSLTPEQWKSHRSYSCQVT | |
| | HEGSTVEKTVAPTECS | |

TAK-079 inhibits the growth of tumor cells expressing CD38 by cell depletion via antibody dependent cellular cytotoxicity (ADCC) and complement dependent cytotoxicity (CDC). TAK-079 also reduces the level of plasma cells and plasmablasts in blood isolated from healthy subjects and systemic lupus erythematosus (SLE) patients. In the case of SLE, 80% of the plasma cell population including short- and long-lived plasma cells, is reduced. Additionally, the number of cells producing pathogenic autoantibodies was also reduced, including VH4-34 9G4+ antibodies (70% reduction), anti-Ro antibody (70% reduction), and anti-dsDNA antibody (80% reduction). The anti-human CD38 mAb daratumumab also depletes CD38-expressing plasmablasts and plasma cells in samples from patients with SLE and RA in a dose-dependent manner in vitro. In contrast to daratumumab, TAK-079 cross-reacts with CD38 expressed by cynomolgus monkeys providing a unique opportunity to determine if reducing the level of cells expressing CD38 would affect inflammation and tissue damage in a non-human primate model of autoimmune disease. In healthy cynomolgus monkeys, the efficiency of depletion for lymphocytes, and B, T and NK cells correlated positively with level of CD38 expression and AB79 dose level (PCT Application No. PCT/US2017/042128; U.S. Pat. No. 8,362,211; incorporated herein by reference in their entirety).

In some embodiments, the anti-CD38 antibodies or antigen binding fragments thereof of the invention interact with CD38 at a number of amino acid residues including K121, F135, Q139, D141, M142, E239, W241, S274, C275, K276, F284, V288, K289, N290, P291, E292, D293 and S294 based on human sequence numbering. Suitably, the anti-CD38 antibodies or antigen binding fragments thereof of the invention may interact with CD38 at a number of amino acid residues including K121, F135, Q139, D141, M142, E239, W241, S274, C275, K276, F284, V288, K289, N290, P291, E292, D293 and S294 of SEQ ID NO: 1, based on human sequence numbering. Suitably, the anti-CD38 antibodies or antigen binding fragments thereof of the invention interact with CD38 at a number of amino acid residues including K121, F135, Q139, D141, M142, E239, W241, F274, C275, K276, F284, V288, K289, N290, P291, E292, D293 and S294 of SEQ ID NO: 2. It should be noted that these residues are identical in both human and cynomolgus monkeys, with the exception that S274 is actually F274 in cynomolgus monkeys. These residues may represent the immunodominant epitope and/or residues within the footprint of the specific antigen binding peptide.

In some embodiments, the anti-CD38 antibody for use according to the invention comprises a heavy chain (HC) comprising the following CDR amino acid sequences: GFTFDDYG (SEQ TD NO:3; HCDR1 TAK-079), ISWNGGKT (SEQ ID NO:4; HCDR2 TAK-079), and ARGSLFHDSSGFYFGH (SEQ ID NO:5; HCDR3 TAK-079) or variants of those sequences having up to three amino acid changes. In some embodiments, the antibody for use according to the invention comprises a light chain (LC) comprising the following CDR amino acid sequences: SSNIGDNY (SEQ ID NO:6; LCDR1 TAK-079), RDS (SEQ ID NO:7; LCDR2 TAK-079), and QSYDSSLSGS (SEQ ID NO:8; LCDR3 TAK-079) or variants of those sequences having up to three amino acid changes. In some embodiments, the antibody for use according to the invention comprises an HC comprising the following CDR amino acid sequences: GFTFDDYG (SEQ ID NO:3; HCDR1 TAK-079), ISWNGGKT (SEQ ID NO:4; HCDR2 TAK-079), ARGSLFHDSSGFYFGH (SEQ ID NO: 5; HCDR3 TAK-079) or variants of those sequences having up to three amino acid changes and an LC comprising the following CDR amino acid sequences: SSNIGDNY (SEQ ID NO:6; LCDR1 TAK-079), RDS (SEQ ID NO:7; LCDR2 TAK-079), and QSYDSSLSGS (SEQ ID NO:8; LCDR3 TAK-079) or variants of those sequences having up to three amino acid changes. In some embodiments, the anti-CD38 antibody comprises an HC comprising the following CDR amino acid sequences: GFTFDDYG (SEQ ID NO:3; HCDR1 TAK-079), ISWNGGKT (SEQ ID NO:4; HCDR2 TAK-079), and ARGSLFHDSSGFYFGH (SEQ ID NO:5; HCDR3 TAK-079). In some embodiments, the antibody comprises an LC comprising the following CDR amino acid sequences: SSNIGDNY (SEQ ID NO:6; LCDR1 TAK-079), RDS (SEQ ID NO:7; LCDR2 TAK-079), and QSYDSSLSGS (SEQ ID NO:8; LCDR3 TAK-079). In some embodiments, the antibody comprises an HC comprising the following CDR amino acid sequences: GFTFDDYG (SEQ ID NO:3; HCDR1 TAK-079), ISWNGGKT (SEQ ID NO:4; HCDR2 TAK-079), ARGSLFHDSSGFYFGH (SEQ ID NO:5; HCDR3 TAK-079) and an LC comprising the following CDR amino acid sequences: SSNIGDNY (SEQ ID NO:6; LCDR1 TAK-079), RDS (SEQ ID NO:7; LCDR2 TAK-079), and QSYDSSLSGS (SEQ ID NO:8; LCDR3 TAK-079). In some embodiments, the antibody comprises an HC comprising a VH region amino acid sequence having at least 80% sequence identity to SEQ ID NO:9. Suitably, the VH region may comprise the CDR sequences as defined by SEQ ID NO: 3, SEQ ID NO: 4 and SEQ ID NO: 5 and the remainder of the VH region sequence may have at least 80% sequence identity to SEQ ID NO: 9. Suitably, the VH region may comprise the CDR sequences as defined by SEQ ID NO: 3, SEQ ID NO: 4 and SEQ ID NO: 5 and the remainder of the VH region sequence may have at least 85% sequence identity to SEQ ID NO: 9. Suitably, the VH region may comprise the CDR sequences as defined by SEQ ID NO: 3, SEQ ID NO: 4 and SEQ ID NO: 5 and the remainder of the VH region sequence may have at least 90% sequence identity to SEQ ID NO: 9. Suitably, the VH region may comprise the CDR sequences as defined by SEQ ID NO: 3, SEQ ID NO: 4 and SEQ ID NO: 5 and the remainder of the VH region sequence may have at least 95% sequence identity to SEQ ID NO: 9. Suitably, the VH region may comprise the CDR sequences as defined by SEQ ID NO: 3, SEQ ID NO: 4 and SEQ ID NO: 5 and the remainder of the VH region sequence may have at least 97% sequence identity to SEQ ID NO: 9. Suitably, the VH region may comprise the CDR sequences as defined by SEQ ID NO: 3, SEQ ID NO: 4 and SEQ ID NO: 5 and the remainder of the VH region sequence may have at least 99% sequence identity to SEQ ID NO: 9.

In some embodiments, the antibody comprises an HC comprising the VH region amino acid sequence of SEQ ID NO:9.

In some embodiments, the antibody comprises an LC comprising a VL region amino acid sequence having at least 80% sequence identity to SEQ ID NO:10. Suitably, the VL region may comprise the CDR sequences as defined by SEQ ID NO: 6, SEQ ID NO: 7 and SEQ ID NO: 8 and the remainder of the VL region sequence may have at least 80% sequence identity to SEQ ID NO: 10. Suitably, the VL region may comprise the CDR sequences as defined by SEQ ID NO: 6, SEQ ID NO: 7 and SEQ ID NO: 8 and the remainder of the VL region sequence may have at least 85% sequence identity to SEQ ID NO: 10. Suitably, the VL region may comprise the CDR sequences as defined by SEQ ID NO: 6, SEQ ID NO: 7 and SEQ ID NO: 8 and the remainder of the VL region sequence may have at least 90% sequence identity to SEQ ID NO: 10. Suitably, the VL region may comprise the CDR sequences as defined by SEQ ID NO: 6, SEQ ID NO: 7 and SEQ ID NO: 8 and the remainder of the VL region sequence may have at least 95% sequence identity to SEQ ID NO: 10. Suitably, the VL region may comprise the CDR sequences as defined by SEQ ID NO: 6, SEQ ID NO: 7 and SEQ ID NO: 8 and the remainder of the VL region sequence may have at least 97% sequence identity to SEQ ID NO: 10. Suitably, the VL region may comprise the CDR sequences as defined by SEQ ID NO: 6, SEQ ID NO: 7 and SEQ ID NO: 8 and the remainder of the VL region sequence may have at least 99% sequence identity to SEQ ID NO: 10.

In some embodiments, the antibody comprises an LC comprising the VL region amino acid sequence of SEQ ID NO:10.

In some embodiments, the antibody comprises an HC comprising the VH region amino acid sequence of SEQ ID NO:9 or a variant thereof as described herein and an LC comprising the VL region amino acid sequence of SEQ ID NO:10 or a variant thereof as described herein.

As will be appreciated by those in the art, the VH and VL regions can be joined to human IgG constant domain sequences, generally IgG1, IgG2 or IgG4.

In some embodiments, the antibody comprises a heavy chain (HC) comprising or consisting of an amino acid sequence having at least 80%, 85%, 90%, 95%, 97% or 99% sequence identity to SEQ ID NO:11. Suitably, the HC may comprise the CDR sequences as defined by SEQ ID NO: 3, SEQ ID NO: 4 and SEQ ID NO: 5 and the remainder of the HC may have at least 80% sequence identity to SEQ ID NO 11. Suitably, the HC may comprise the CDR sequences as defined by SEQ ID NO: 3, SEQ ID NO: 4 and SEQ ID NO: 5 and the remainder of the HC may have at least 85% sequence identity to SEQ ID NO 11. Suitably, the HC may comprise the CDR sequences as defined by SEQ ID NO: 3, SEQ ID NO: 4 and SEQ ID NO: 5 and the remainder of the HC may have at least 90% sequence identity to SEQ ID NO 11. Suitably, the HC may comprise the CDR sequences as defined by SEQ ID NO: 3, SEQ ID NO: 4 and SEQ ID NO: 5 and the remainder of the HC may have at least 95% sequence identity to SEQ ID NO 11. Suitably, the HC may comprise the CDR sequences as defined by SEQ ID NO: 3, SEQ ID NO: 4 and SEQ ID NO: 5 and the remainder of the HC may have at least 97% sequence identity to SEQ ID NO 11. Suitably, the HC may comprise the CDR sequences as defined by SEQ ID NO: 3, SEQ ID NO: 4 and SEQ ID NO: 5 and the remainder of the HC may have at least 99% sequence identity to SEQ ID NO 11.

In some embodiments, the antibody comprises the HC amino acid sequence of SEQ ID NO:11. In some embodiments, the antibody comprises a light chain (LC) comprising or consisting of an amino acid sequence having at least 80%, 85%, 90%, 95%, 97% or 99% sequence identity to SEQ ID NO:12. Suitably, the LC may comprise the CDR sequences as defined by SEQ ID NO: 6, SEQ ID NO: 7 and SEQ ID NO: 8 and the remainder of the LC may have at least 80% sequence identity to SEQ ID NO 12. Suitably, the LC may comprise the CDR sequences as defined by SEQ ID NO: 6, SEQ ID NO: 7 and SEQ ID NO: 8 and the remainder of the LC may have at least 85% sequence identity to SEQ ID NO 12. Suitably, the LC may comprise the CDR sequences as defined by SEQ ID NO: 6, SEQ ID NO: 7 and SEQ ID NO: 8 and the remainder of the LC may have at least 90% sequence identity to SEQ ID NO 12. Suitably, the LC may comprise the CDR sequences as defined by SEQ ID NO: 6, SEQ ID NO: 7 and SEQ ID NO: 8 and the remainder of the LC may have at least 95% sequence identity to SEQ ID NO 12. Suitably, the LC may comprise the CDR sequences as defined by SEQ ID NO: 6, SEQ ID NO: 7 and SEQ ID NO: 8 and the remainder of the LC may have at least 97% sequence identity to SEQ ID NO 12. Suitably, the LC may comprise the CDR sequences as defined by SEQ ID NO: 6, SEQ ID NO: 7 and SEQ ID NO: 8 and the remainder of the LC may have at least 99% sequence identity to SEQ ID NO 12.

In some embodiments, the antibody comprises the LC amino acid sequence of SEQ ID NO:12.

In some embodiments, the antibody comprises or consists of the HC amino acid sequence of SEQ ID NO:11 or a variant thereof as described herein and the LC amino acid sequence of SEQ ID NO:12 or a variant thereof as described herein.

The present invention encompasses antibodies that bind to both human and cyno CD38 and interact with at least 80%, 85%, 90%, 91%, 92%, 93%, 94%, 95%, 96%, 97%, 98% or 99% of the following amino acid residues: K121, F135, Q139, D141, M142, E239, W241, S274, C275, K276, F284, V288, K289, N290, P291, E292, D293 and S294 of SEQ ID NO: 1 and SEQ ID NO: 2, based on human numbering. Suitably, the antibody may interact with at least 90% of these amino acid residues. Suitably, the antibody may interact with at least 95% of these amino acid residues. Suitably, the antibody may interact with at least 97% of these amino acid residues. Suitably, the antibody may interact with at least 98% of these amino acid residues. Suitably, the antibody may interact with at least 99% of these amino acid residues. Suitably, the antibody may interact with at least 14 (e.g., at least 15 or at least 16) of the following amino acids: K121, F135, Q139, D141, M142, E239, W241, S274, C275, K276, F284, V288, K289, N290, P291, E292, D293 and S294 of SEQ ID NO: 1 and SEQ ID NO: 2, based on human numbering.

In some embodiments, the antibodies are full length. By “full length antibody” herein is meant the structure that constitutes the natural biological form of an antibody, including variable and constant regions, including one or more modifications as outlined herein.

Alternatively, the antibodies can be a variety of structures, including, but not limited to, antibody fragments, antigen binding fragment, monoclonal antibodies, bispecific antibodies, minibodies, domain antibodies, synthetic antibodies (sometimes referred to herein as “antibody mimetics”), chimeric antibodies, humanized antibodies, antibody fusions (sometimes referred to as “antibody conjugates”), and fragments of each, respectively. Specific antibody fragments include, but are not limited to, (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) the dAb fragment (Ward et al. (1989) Nature 341: 544-546) which consists of a single variable, (v) isolated CDR regions, (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al. (1988) Science 242: 423-426, Huston et al. (1988) Proc. Natl. Acad. Sci. USA 85: 5879-5883), (viii) bispecific single chain Fv (WO 03/11161) and (ix) “diabodies” or “triabodies”, multivalent or multispecific fragments constructed by gene fusion (Tomlinson et al. (2000) Methods Enzymol. 326: 461-479; WO94/13804; Holliger et al. (1993) Proc. Natl. Acad. Sci. USA 90: 6444-6448).

Suitably, the antibody may be a Fab fragment. Suitably, the antibody may be an Fv fragment. Suitably, the antibody may be an Fd fragment. Suitably, the antibody structure may be isolated CDR regions. Suitably, the antibody may be a F(ab′)2 fragment. Suitably, the antibody may be an scFv fragment.

In some embodiments, the antibody or antigen binding fragment thereof of the present invention further comprises one or more engineered glycoforms. In some embodiments, the engineered glycoform comprises glycosylation of one or more polypeptides. In some embodiments, the glycosylation is N-linked glycosylation or O-linked glycosylation. In some embodiments, the glycosylation is N-linked glycosylation. In some embodiments, the glycosylation is O-linked glycosylation.

In some embodiments, the isolated antibody of the present invention is Mezagitamab.

### Antibody Modifications

The present invention further provides variant anti-CD38 antibodies. That is, there are a number of modifications that can be made to the antibodies of the invention, including, but not limited to, amino acid modifications in the CDRs (affinity maturation), amino acid modifications in the VH region and/or VL region, amino acid modifications in the HC and/or LC, amino acid modifications in the Fc region, glycosylation variants, covalent modifications of other types, etc.

The term “variant” means a polypeptide that differs from that of a parent polypeptide. Amino acid variants can include substitutions, insertions and deletions of amino acids. In general, variants can include any number of modifications, as long as the function of the protein is still present, as described herein. That is, in the case of amino acid variants generated with the CDRs of TAK-079, for example, the antibody should still specifically bind to both human and cynomolgus CD38. The term “variant Fc region” means an Fc sequence that differs from that of a wild-type or parental Fc sequence by virtue of at least one amino acid modification. Fc variant may refer to the Fc polypeptide itself, compositions comprising the Fc variant polypeptide, or the amino acid sequence. If amino acid variants are generated with the Fc region, for example, the variant antibodies should maintain the required functions for the particular application or indication of the antibody. For example, 1, 2, 3, 4, 5, 6, 7, 8, 9 or 10 amino acid substitutions can be utilized, for example, 1-10, 1-5, 1-4, 1-3, and 1-2 substitutions. Suitable modifications can be made at one or more positions as is generally outlined, for example in U.S. patent application Ser. Nos. 11/841,654; 12/341,769; US Patent Publication Nos. 2004013210; 20050054832; 20060024298; 20060121032; 20060235208; 20070148170; and U.S. Pat. Nos. 6,737,056; 7,670,600; and 6,086,875, all of which are expressly incorporated by reference in their entirety, and in particular for specific amino acid substitutions that increase binding to Fc receptors.

A variant can be considered in terms of similarity (i.e., amino acid residues having similar chemical properties/functions), preferably a variant is expressed in terms of sequence identity.

Sequence comparisons can be conducted by eye, or more usually, with the aid of readily available sequence comparison programs. These publicly and commercially available computer programs can calculate sequence identity between two or more sequences.

It may be desirable to have from 1-5 modifications in the Fc region of wild-type or engineered proteins, as well as from 1 to 5 modifications in the Fv region, for example. A variant polypeptide sequence will preferably possess at least about 80%, 85%, 90%, 91%, 92%, 93%, 94%, 95%, 96%, 97%, 98% or 99% identity to the parent sequences (e.g., the VH or VL regions, the constant regions, and/or the HC and LC sequences for TAK-079). Suitably, the variant may have at least 80% sequence identity to the parent sequence. Suitably, the variant may have at least 85% sequence identity to the parent sequence. Suitably, the variant may have at least 90% sequence identity to the parent sequence. Suitably, the variant may have at least 92% sequence identity to the parent sequence. Suitably, the variant may have at least 95% sequence identity to the parent sequence. Suitably, the variant may have at least 97% sequence identity to the parent sequence. Suitably, the variant may have at least 98% sequence identity to the parent sequence. Suitably, the variant may have at least 99% sequence identity to the parent sequence.

In one embodiment, the sequence identity is determined across the entirety of the sequence. In one embodiment, the sequence identity is determined across the entirety of the candidate sequence being compared to a sequence recited herein.

The term “amino acid substitution” means the replacement of an amino acid at a particular position in a parent polypeptide sequence with another amino acid. For example, the substitution S100A refers to a variant polypeptide in which the serine at position 100 is replaced with alanine. Suitably the amino acid substitution may be a conservative amino acid substitution. Suitably a variant may comprise one or more, e.g., two or three conservative amino acid substitutions. Amino acids with similar biochemical properties may be defined as amino acids which can be substituted via a conservative substitution.

Unless otherwise explicitly stated herein by way of reference to a specific, individual amino acid, amino acids may be substituted using conservative substitutions as recited below. An aliphatic, polar uncharged amino may be a cysteine, serine, threonine, methionine, asparagine or glutamine residue. An aliphatic, polar charged amino acid may be an aspartic acid, glutamic acid, lysine or arginine residue. An aromatic amino acid may be a histidine, phenylalanine, tryptophan or tyrosine residue. Conservative substitutions may be made, for example according to Table 3 below. Amino acids in the same block in the second column and preferably in the same line in the third column may be substituted for each other:

TABLE 3
Conservative Substitutions

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The term “amino acid insertion” means the addition of an amino acid at a particular position in a parent polypeptide sequence.

The term “amino acid deletion” means the removal of an amino acid at a particular position in a parent polypeptide sequence.

The terms “parent antibody” and “precursor antibody” mean an unmodified antibody that is subsequently modified to generate a variant. In an embodiment, the parent antibody herein is TAK-079. In an embodiment, the parent antibody herein comprises a VH region having the amino acid sequence of SEQ ID NO: 9 and the VL region having the amino acid sequence of SEQ ID NO: 10. In an embodiment, the parent antibody herein comprises an HC amino acid sequence of SEQ ID NO: 11 and an LC amino acid sequence of SEQ ID NO: 12. Parent antibody may refer to the polypeptide itself, compositions that comprise the parent antibody, or the amino acid sequence that encodes it. Accordingly, the term “parent Fc polypeptide” means an Fc polypeptide that is modified to generate a variant.

The terms “wild type,” “WT,” and “native” mean an amino acid sequence or a nucleotide sequence that is found in nature, including allelic variations. A WT protein, polypeptide, antibody, immunoglobulin, IgG, etc., has an amino acid sequence or a nucleotide sequence that has not been intentionally modified.

In some embodiments, one or more amino acid modifications are made in one or more of the CDRs of the anti-CD38 antibody. In general, only 1, 2, or 3 amino acids are substituted in any single CDR, and generally no more than from 4, 5, 6, 7, 8 9 or 10 changes are made within a set of CDRs. However, it should be appreciated that any combination of no substitutions, 1, 2 or 3 substitutions in any CDR can be independently and optionally combined with any other substitution.

In some cases, amino acid modifications in the CDRs are referred to as “affinity maturation”. An “affinity matured” antibody is one having one or more alteration(s) in one or more CDRs which results in an improvement in the affinity of the antibody for antigen, compared to a parent antibody which does not possess those alteration(s). In some cases, it may be desirable to decrease the affinity of an antibody to its antigen.

Affinity maturation can be done to increase the binding affinity of the antibody for the antigen by at least about 10% to 50%, 100%, 150% or more, or from 1- to 5-fold as compared to the “parent” antibody. Preferred affinity matured antibodies will have nanomolar or even picomolar affinities for the target antigen. Affinity matured antibodies are produced by known procedures (e.g., Marks et al. (1992) Biotechnol. 10: 779-783; Barbas et al. (1994) Proc. Nat. Acad. Sci. USA 91: 3809-3813; Shier et al. (1995) Gene 169: 147-155; Yelton et al. (1995) J Immunol. 155: 1994-2004; Jackson et al. (1995).J. Immunol. 154(7): 3310-9; and Hawkins et al. (1992) J. Mol. Biol. 226: 889-896; incorporated herein by reference in their entirety).

Alternatively, amino acid modifications can be made, e.g., in one or more of the CDRs of the antibodies of the invention that are “silent”, e.g., that do not significantly alter the affinity of the antibody for the antigen. These can be made for a number of reasons, including optimizing expression (as can be done for the nucleic acids encoding the antibodies of the invention).

Thus, included within the definition of the CDRs and antibodies of the invention are variant CDRs and antibodies; that is, the antibodies of the invention can include amino acid modifications in one or more of the CDRs set forth in SEQ ID NO:3 to 8. In addition, as outlined below, amino acid modifications can also independently and optionally be made in any region outside the CDRs, including framework and constant regions.

In some embodiments, variant antibodies of TAK-079 that are specific for human CD38 (SEQ ID NO:1) and cynomolgus CD38 (SEQ ID NO:2) is described. This antibody is composed of six CDRs, wherein each CDR of this antibody can differ from SEQ ID NO:3, SEQ ID NO:4, SEQ ID NO:5, SEQ ID NO:6, SEQ ID NO:7, and/or SEQ ID NO:8 by 0, 1, or 2 amino acid substitutions.

### Glycosylation

Another type of modification is alterations in glycosylation. In one embodiment, the antibodies disclosed herein can be modified to include one or more engineered glycoforms. By “engineered glycoform” as used herein is meant a carbohydrate composition that is covalently attached to the antibody, wherein said carbohydrate composition differs chemically from that of a parent antibody. Engineered glycoforms may be useful for a variety of purposes, including but not limited to enhancing or reducing effector function. A preferred form of engineered glycoform is afucosylation, which has been shown to be correlated to an increase in ADCC function, presumably through tighter binding to the FcγRIIIa receptor. In this context, “afucosylation” means that the majority of the antibody produced in the host cells is substantially devoid of fucose, e.g., 90-95-98% of the generated antibodies do not have appreciable fucose as a component of the carbohydrate moiety of the antibody (generally attached at N297 in the Fc region). Defined functionally, afucosylated antibodies generally exhibit at least a 50% or higher affinity to the FcγRIIIa receptor.

Engineered glycoforms may be generated by a variety of methods known in the art (U.S. Pat. No. 8,362,211; incorporated herein by reference in its entirety). Engineered glycoform typically refers to the different carbohydrate or oligosaccharide; thus, an antibody can include an engineered glycoform.

Alternatively, engineered glycoform may refer to the IgG variant that comprises the different carbohydrate or oligosaccharide. As is known in the art, glycosylation patterns can depend on both the sequence of the protein (e.g., the presence or absence of particular glycosylation amino acid residues, discussed below), or the host cell or organism in which the protein is produced. Particular expression systems are discussed below.

Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the antibody amino acid sequence is preferably altered through changes at the DNA level, particularly by mutating the DNA encoding the target polypeptide at preselected bases such that codons are generated that will translate into the desired amino acids.

Another means of increasing the number of carbohydrate moieties on the antibody is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, € aromatic residues such as those of phenylalanine, tyrosine, or tryptophan, or (f) the amide group of glutamine. These methods are described in WO87/05330 and in Aplin and Wriston, 1981, CRC Crit. Rev. Biochem., pp. 259-306, both entirely incorporated by reference.

Removal of carbohydrate moieties present on the starting antibody (e.g., post-translationally) may be accomplished chemically or enzymatically. Chemical deglycosylation requires exposure of the protein to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al., 1987, Arch. Biochem. Biophys. 259:52 and by Edge et al., 1981, Anal. Biochem. 118: 131, both entirely incorporated by reference. Enzymatic cleavage of carbohydrate moieties on polypeptides can be achieved by the use of a variety of endo- and exo-glycosidases as described by Thotakura et al., 1987, Meth. Enzymol. 138:350, entirely incorporated by reference. Glycosylation at potential glycosylation sites may be prevented by the use of the compound tunicamycin as described by Duskin et al., 1982, J. Biol. Chem. 257:3105, entirely incorporated by reference. Tunicamycin blocks the formation of protein-Nglycoside linkages.

Another type of covalent modification of the antibody comprises linking the antibody to various nonproteinaceous polymers, including, but not limited to, various polyols such as polyethylene glycol, polypropylene glycol or polyoxyalkylenes, in the manner set forth in, for example, 2005-2006 PEG Catalog from Nektar Therapeutics (available at the Nektar website) U.S. Pat. Nos. 4,640,835; 4,496,689; 4,301,144; 4,670,417; 4,791,192 or 4,179,337, all entirely incorporated by reference. In addition, as is known in the art, amino acid substitutions may be made in various positions within the antibody to facilitate the addition of polymers such as PEG. See for example, U.S. Publication No. 2005/0114037A1, entirely incorporated by reference.

In addition to the modifications outlined above, other modifications can be made. For example, the molecules may be stabilized by the incorporation of disulphide bridges linking the VH and VL domains (Reiter et al. (1996) Nature Biotech. 14: 1239-1245; incorporated herein by reference in its entirety). In addition, there are a variety of covalent modifications of antibodies that can be made as outlined below.

Covalent modifications of antibodies are included within the scope of this invention, and are generally, but not always, done post-translationally. For example, several types of covalent modifications of the antibody are introduced into the molecule by reacting specific amino acid residues of the antibody with an organic derivatizing agent that is capable of reacting with selected side chains or the N- or C-terminal residues.

In some embodiments, the anti-CD38 antibody of the present invention specifically binds to one or more residues or regions in CD38 but also does not cross-react with other proteins with homology to CD38, such as BST-1 (bone marrow stromal cell antigen-1) and/or Mo5, also called CD157.

Typically, a lack of cross-reactivity means less than about 5% relative competitive inhibition between the molecules when assessed by ELISA and/or FACS analysis using sufficient amounts of the molecules under suitable assay conditions.

### Side Effect Reduction

An adverse event (AE) was defined as any untoward medical occurrence in a clinical investigation subject administered a drug; it did not necessarily have to have a causal relationship with this treatment. Treatment-emergent adverse events (TEAEs) were defined as AEs that occurred after the first dose of study drug received in the treatment period and until the end of safety follow-up. The terms “serious TEAEs” and “treatment-emergent SAEs” and can be considered interchangeable in this document. PTE and AE verbatim terms were coded by SOC and PT using MedDRA version 24.0. TEAEs are typically referred to by grades 1, 2, 3, 4, and 5, grade 1 being the least severe and grade 5 being the most severe TEAE. Based on FDA and other guidelines for Common Terminology Criteria for Adverse Events (CTCAE) standards for oncology drugs (see, e.g., https://evs.nci.nih.gov/ftpl/CTCAE/CTCAE_4.03_2010-06-14_QuickReference_5x7.pdf; as well as

https://ctep.cancer.gov/protocoldevelopment/electronic_applications/ctc.htm; and Nilsson and Koke (2001) DrugInform. J. 35: 1289-1299; incorporated herein by reference in its entirety) the following is how such grades are generally determined. Grade 1 is mild: asymptomatic or mild symptoms; clinical or diagnostic observations only; no intervention indicated. Grade 2 is moderate: minimal, local or noninvasive intervention indicated; limiting age-appropriate instrumental activities of daily living (“ADL”). Grade 3 is severe or medically significant but not immediately life-threatening: hospitalization or prolongation of hospitalization indicated; disabling; limiting self-care ADL. Grade 4 is life-threatening consequence: urgent intervention indicated. Grade 5 is death related to AE.

The anti-CD38 antibodies of the present invention allow for reduced side effects compared to prior art anti-CD38 antibodies. In some embodiments, the antibody for use according to the present invention, e.g., TAK-079 does not induce TEAEs. In some embodiments, the antibody for use according to the present invention, e.g., TAK-079 allows for a reduction in the incidence of TEAEs in a patient population as compared to other anti-CD38 antibodies, such as MOR202. In some embodiments, the antibody for use according to the present invention, e.g., TAK-079 allows for a reduction in the grade of the TEAEs in a patient population as compared to other anti-CD38 antibodies, such as MOR202. In some embodiments, the antibody for use according to the present invention, e.g., TAK-079 allows for a reduction in the grade of the TEAEs as compared to other anti-CD38 antibodies from grade 5 to grade 4. In some embodiments, the antibody for use according to the present invention, e.g., TAK-079 allows for a reduction in the grade of the TEAEs as compared to other anti-CD38 antibodies from grade 4 to grade 3. In some embodiments, the antibody for use according to the present invention, e.g., TAK-079 allows for a reduction in the grade of the TEAEs as compared to other anti-CD38 antibodies from grade 3 to grade 2. In some embodiments, the antibody for use according to the present invention, e.g., TAK-079 allows for a reduction in the grade of the TEAEs as compared to other anti-CD38 antibodies from grade 2 to grade 1.

In some embodiments, the antibody for use according to the present invention, e.g., TAK-079 allows for a reduction in grade of one or more TEAEs selected from the group consisting of anemia (including hemolytic anemia), thrombocytopenia, fatigue, infusion-related reactions (IRRs), leukopenia, lymphopenia, and nausea. In some embodiments, the antibody for use according to the present invention, e.g., TAK-079 allows for a reduction in the occurrence of one or more TEAEs selected from the group consisting of anemia (including hemolytic anemia), thrombocytopenia, fatigue, infusion-related reactions (IRRs), leukopenia, lymphopenia, and nausea.

In some embodiments, administering the antibody or antigen binding fragment thereof of the present invention results in less than 10% incidence of grade 3 or 4 of one or more TRAEs or TEAEs; optionally wherein the TRAEs or TEAEs are selected from the group consisting of gastrointestinal disorders, nausea, infestations, pyrexia, Herpes Zoster, urinary tract infection, skin and cutaneous tissue disorders, headache, fever, chills/rigors, vomiting, diarrhea, arthralgia, myalgia, hypotension, respiratory, thoracic and mediastinal disorders, thrombocytopenia, leukopenia, lymphopenia, cardiac disorders, palpitations, and dyspnea.

In some embodiments, administering the antibody or antigen binding fragment thereof of the present invention results in one or more TRAEs or TEAEs having a maximum intensity of Common Terminology Criteria for Adverse Events (CTCAE) Grade 1 or Grade 2.

### Disease Indication

The antibodies or antigen binding fragment thereof, methods, and dosage units of the invention find use in treating patients with systemic lupus erythematosus (SLE), especially in treating patients with moderate or severe SLE.

### Systemic Lupus Erythematosus (SLE)

SLE is a heterogeneous autoimmune disease that is characterized by dysregulation of T and B lineage cells as well as other components of the innate immune system, production of autoantibodies and the formation of immune complexes resulting in damage of multiple organs and variable clinical symptoms. SLE is defined by either the 2012 Systemic Lupus International Collaborating Clinics (SLICC) (https://sliccgroup.org/research/sle-criteria/) or the American College of Rheumatology diagnostic criteria.

The 2012 SLICC criteria for SLE classification requires: 1) Fulfillment of at least four criteria, with at least one clinical criterion AND one immunologic criterion OR 2) Lupus nephritis as the sole clinical criterion in the presence of ANA or anti-dsDNA antibodies.

Clinical Criteria: (1) acute cutaneous lupus; (2) chronic cutaneous lupus; (3) oral ulcers: palate; (4) nonscarring alopecia (diffuse thinning or hair fragility with visible broken hairs); (5) synovitis involving two or more joints, characterized by swelling or effusion OR tenderness in two or more joints and thirty minutes or more of morning stiffness; (6) serositis; (7) renal; (8) neurologic; (9) hemolytic anemia; (10) leukopenia (<4000/mm³ at least once); or (11) thrombocytopenia (<100,000/mm³) at least once.

Immunological Criteria: (1) antinuclear antibodies (ANAs) above laboratory reference range; (2) anti-dsDNA above laboratory reference range, except ELISA (Anti-Sm or Anti-Smith); (3) antiphospholipid antibody, (4) low complement; or (5) direct Coombs test in the absence of hemolytic anemia.

### Moderate to Severe SLE

A subject diagnosed with “moderate SLE” is defined as a subject having a SLEDAI-2K score between 6 and 8.

A subject diagnosed with “severe SLE” is defined as a subject having a SLEDAI-2K score between 9 and 12.

The SLEDAI-2K disease assessment tool uses 24 items, of which 16 are clinical and 8 are based solely on laboratory results (urinary casts, hematuria, proteinuria, pyuria, low complement levels, increased DNA binding, thrombocytopenia, and leukopenia). See, e.g., Gladman et al. (2002). The Journal of rheumatology, 29(2):288-291.

Treating and identifying novel therapies for SLE is challenging because of its genetic and phenotypic heterogeneity, leaving a significant unmet medical need for patients especially with moderate to severe SLE (Kiriakidou and Ching (2020) Ann. Intern. Med. 172(11): ITC81-ITC96, incorporated herein by reference in its entirety).

Studies have shown an increased number of PBs in the blood of patients with active SLE (D'Cruz (2006) Blood (ASH Annual Meeting Abstracts) 129: 2359-67; Clemens et al. (2017) Clin. Pharmacokinet. 56(8): 915-24; Sanada et al. (2016) Blood 128(7): 923-33; Wang et al. (2016) Arthrit. Rheumatol. 68(suppl 10):1085; incorporated by reference in their entirety). CD38 is a type 11 glycoprotein that is highly and uniformly expressed on antibody-producing PBs and plasma cells (Sullivan et al. (2017) Blood 129(22): 3033-7; incorporated herein by reference in its entirety), making it a potential target for treatment of SLE. In an ex vivo study of CD38 expression on various immune cells in peripheral blood mononuclear cells from patients with SLE, the highest CD38 expression was observed on plasma cells and PBs, followed by natural killer (NK) cells, plasmacytoid dendritic cells, a regulatory T cell subpopulation, and naïve T cells (Ramaschi et al. (1996) Blood 87(6):2308-13; incorporated herein by reference in its entirety). The significantly higher CD38 expression on plasma cells and PBs compared with other immune cells suggests the potential for selectively depleting these cells with an anti-CD38 antibody.

The therapeutic anti-CD38 antibodies of the present invention bind to CD38 positive cells, resulting in depletion of these cells through multiple mechanisms of action, including both CDC and ADCC pathways.

In some embodiments, the invention provides methods of treating moderate or severe SLE in a subject, the method comprising administering to the subject an isolated human anti-CD38 antibody or antigen binding fragment thereof, wherein the antibody or antigen binding fragment thereof comprises: (a) a VH region comprising a CDR1 having the amino acid sequence of SEQ ID NO:3, a CDR2 having the amino acid sequence of SEQ ID NO:4, and a CDR3 having the amino acid sequence of SEQ ID NO:5; and (b) a VL region comprising a CDR1 having the amino acid sequence of SEQ ID NO:6, a CDR2 having the amino acid sequence of SEQ ID NO:7, and a CDR3 having the amino acid sequence of SEQ ID NO:8; and wherein the antibody or antigen binding fragment thereof is subcutaneously administered in a dosage of from 40 milligrams to 140 milligrams.

In some embodiments, the invention provides methods of reducing the level of plasmablasts and/or plasma cells in a subject diagnosed with moderate or severe SLE, the method comprising administering to the subject an isolated human anti-CD38 antibody or antigen binding fragment thereof, wherein the antibody or antigen binding fragment thereof comprises: (a) a VH region comprising a CDR1 having the amino acid sequence of SEQ ID NO:3, a CDR2 having the amino acid sequence of SEQ ID NO:4, and a CDR3 having the amino acid sequence of SEQ ID NO:5; and (b) a VL region comprising a CDR1 having the amino acid sequence of SEQ ID NO:6, a CDR2 having the amino acid sequence of SEQ ID NO:7, and a CDR3 having the amino acid sequence of SEQ ID NO:8; and wherein the antibody or antigen binding fragment thereof is subcutaneously administered in a dosage of from 40 milligrams to 140 milligrams.

In some embodiments, the invention provides methods of reducing the level of immunoglobulin(s) in a subject diagnosed with moderate or severe SLE, the method comprising administering to the subject an isolated human anti-CD38 antibody or antigen binding fragment thereof, wherein the antibody or antigen binding fragment thereof comprises: a VH region comprising a CDR1 having the amino acid sequence of SEQ ID NO:3, a CDR2 having the amino acid sequence of SEQ ID NO:4, and a CDR3 having the amino acid sequence of SEQ ID NO:5; and a VL region comprising a CDR1 having the amino acid sequence of SEQ ID NO:6, a CDR2 having the amino acid sequence of SEQ ID NO:7, and a CDR3 having the amino acid sequence of SEQ ID NO:8; and wherein the antibody or antigen binding fragment thereof is subcutaneously administered in a dosage of from 40 milligrams to 140 milligrams.

In some embodiments, the invention provides the methods as disclosed herein, wherein the immunoglobulin is IgA, IgG and/or IgM. In some embodiments, the immunoglobulin is IgA.

In some embodiments, the immunoglobulin is IgG. In some embodiments, the immunoglobulin is IgM.

In some embodiments, the invention provides methods of reducing the level of one or more autoantibodies in a subject diagnosed with moderate or severe SLE, the method comprising administering to the subject an isolated human anti-CD38 antibody or antigen binding fragment thereof, wherein the antibody or antigen binding fragment thereof comprises: (a) a VH region comprising a CDR1 having the amino acid sequence of SEQ ID NO:3, a CDR2 having the amino acid sequence of SEQ ID NO:4, and a CDR3 having the amino acid sequence of SEQ ID NO:5; and (b) a VL region comprising a CDR1 having the amino acid sequence of SEQ ID NO:6, a CDR2 having the amino acid sequence of SEQ ID NO:7, and a CDR3 having the amino acid sequence of SEQ ID NO:8; and wherein the antibody or antigen binding fragment thereof is subcutaneously administered in a dosage of from 40 milligrams to 140 milligrams.

In some embodiments, the invention provides the methods as disclosed herein, wherein the one or more autoantibodies is selected from the group consisting of anti-dsDNA, anti-SmDp, beta-2 glycoprotein 1 IgM, ribonucleoprotein-70, Sjogrens SS-A and Sjogrens SS-B.

In some embodiments, the invention provides the methods as disclosed herein, wherein the subject is diagnosed with severe SLE.

In some embodiments, the invention provides the methods as disclosed herein, wherein the antibody or antigen binding fragment thereof further comprises one or more engineered glycoforms.

In some embodiments, the invention provides the methods as disclosed herein, wherein the engineered glycoform comprises glycosylation of one or more polypeptides, and wherein the glycosylation is N-linked glycosylation or O-linked glycosylation.

In some embodiments, the invention provides the methods as disclosed herein, wherein the glycosylation is N-linked glycosylation.

In some embodiments, the invention provides the methods as disclosed herein, wherein the glycosylation is O-linked glycosylation.

In some embodiments, the invention provides the methods as disclosed herein, wherein the VH region of the antibody or antigen binding fragment thereof comprises an amino acid sequence having an identity of at least 90% to SEQ ID NO:9, and/or the VL region of the antibody or antigen binding fragment thereof comprises an amino acid sequence having an identity of at least 90% to SEQ ID NO:10.

In some embodiments, the invention provides the methods as disclosed herein, wherein the VH region comprises an amino acid sequence having an identity of at least 95% to SEQ ID NO:9.

In some embodiments, the invention provides the methods as disclosed herein, wherein the VL region comprises an amino acid sequence having an identity of at least 95% to SEQ ID NO:10.

In some embodiments, the invention provides the methods as disclosed herein, wherein the VH region comprises an amino acid sequence having an identity of at least 99% to SEQ ID NO:9.

In some embodiments, the invention provides the methods as disclosed herein, wherein the VL region comprises an amino acid sequence having an identity of at least 99% to SEQ ID NO:10.

In some embodiments, the invention provides the methods as disclosed herein, wherein the HC of the antibody or antigen binding fragment thereof comprises an amino acid sequence having an identity of at least 95% to SEQ ID NO:11.

In some embodiments, the invention provides the methods as disclosed herein, wherein the LC of the antibody or antigen binding fragment thereof comprises an amino acid sequence having an identity of at least 95% to SEQ ID NO:12.

In some embodiments, the invention provides the methods as disclosed herein, wherein the isolated antibody or antigen binding fragment thereof interacts with at least K121, F135, Q139, D141, E239, W241, C275, K276, F284, P291 and E292 of SEQ ID NO:1 and SEQ ID NO:2, based on human sequence numbering.

In some embodiments, the invention provides the methods as disclosed herein, wherein the isolated antibody or antigen binding fragment thereof binds to human CD38 (SEQ ID NO:1) with a KD of 10⁻⁸ M or a greater affinity, and wherein the affinity is measured by a standard Biacore assay.

In some embodiments, the invention provides the methods as disclosed herein, wherein the VH region comprises SEQ ID NO:9 and the VL region comprises SEQ ID NO:10.

In some embodiments, the invention provides the methods as disclosed herein, wherein the isolated antibody or antigen binding fragment thereof comprises an HC as set forth in SEQ ID NO:11 and an LC as set forth in SEQ ID NO:12.

In some embodiments, the invention provides the methods as disclosed herein, wherein the antibody or antigen binding fragment thereof further comprises an Fc domain.

In some embodiments, the invention provides the methods as disclosed herein, wherein the Fc domain is a human Fc domain. In some embodiments, the Fc domain is a variant Fc domain.

In some embodiments, the invention provides the methods as disclosed herein, wherein the isolated antibody or antigen binding fragment is a human IgG antibody. In some embodiments, the human IgG antibody is a human IgG1 antibody.

In some embodiments, the invention provides the methods as disclosed herein, wherein the subject receives background SLE medication(s).

In some embodiments, the invention provides the methods as disclosed herein, wherein the background SLE medication(s) is selected from the group consisting of immunosuppressants, steroids and immunoglobulin.

In some embodiments, the invention provides the methods as disclosed herein, wherein the background SLE medication(s) is selected from the group consisting of hydroxychloroquine, hydroxychloroquine sulfate, prednisone, methylprednisolone, gabapentin, mycophenolate mofetil, and/or mycophenolic acid.

In some embodiments, the invention provides the methods as disclosed herein, wherein the background SLE medication(s) is administered in combination with the antibody or antigen binding fragment thereof.

In some embodiments, the invention provides the methods as disclosed herein, wherein the antibody or antigen binding fragment thereof is administered in a dosage selected from the group consisting of about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, and about 140 mg; In some embodiments, the antibody or antigen binding fragment thereof is administered in a dosage of about 45 mg. In some embodiments, wherein the antibody or antigen binding fragment thereof is administered in a dosage of about 90 mg. In some embodiments, wherein the antibody or antigen binding fragment thereof is administered in a dosage of about 135 mg.

In some embodiments, the invention provides the methods as disclosed herein, wherein the dosage is a dosage administered once every week, once every two weeks, once every three weeks or once every four weeks.

In some embodiments, the invention provides the methods as disclosed herein, wherein the antibody or antigen binding fragment thereof is administered in the form of a pharmaceutically acceptable composition.

In some embodiments, the invention provides the methods as disclosed herein, wherein the pharmaceutically acceptable composition comprises the isolated antibody or antibody fragment thereof and at least one pharmaceutically acceptable carrier, excipient or stabilizer.

In some embodiments, the invention provides the methods as disclosed herein, wherein the isolated antibody or antigen binding fragment thereof comprises an HC as set forth in SEQ ID NO:11 and an LC as set forth in SEQ ID NO:12; and wherein the antibody or antigen binding fragment thereof is subcutaneously administered once every 3 weeks for 12 weeks. In some embodiments, the invention provides the methods as disclosed herein, wherein the isolated antibody or antigen binding fragment thereof further comprises one or more engineered glycoforms, wherein the engineered glycoform comprises glycosylation of one or more polypeptides and the glycosylation is N-linked glycosylation.

In some embodiments, the invention provides the methods as disclosed herein, wherein the isolated antibody or antigen binding fragment thereof is mezagitamab.

In some embodiments, the invention provides the methods as disclosed herein, wherein administering the antibody or antigen binding fragment thereof results in less than 10% incidence of grade 3 or 4 of one or more treatment-related adverse events (TRAEs) or treatment-emergent adverse events (TEAEs). In some embodiments, the TRAEs or TEAEs are selected from the group consisting of gastrointestinal disorders, nausea, infestations, pyrexia, Herpes Zoster, urinary tract infection, skin and cutaneous tissue disorders, headache, fever, chills/rigors, vomiting, diarrhea, arthralgia, myalgia, hypotension, respiratory, thoracic and mediastinal disorders, thrombocytopenia, leukopenia, lymphopenia, cardiac disorders, palpitations, and dyspnea.

In some embodiments, the invention provides the methods as disclosed herein, wherein administering the antibody or antigen binding fragment thereof results in one or more TRAEs or TEAEs having a maximum intensity of Common Terminology Criteria for Adverse Events (CTCAE) Grade 1 or Grade 2.

### Antibody Compositions for In Vivo Administration

Formulations of the antibodies or antigen binding fragments thereof used in accordance with the present invention are prepared for storage by mixing an antibody having the desired degree of purity with optional pharmaceutically acceptable carriers, excipients or stabilizers (Remington's Pharmaceutical Sciences 16th edition (1980) Osol, A. Ed.; incorporated herein by reference in its entirety), in the form of lyophilized formulations or aqueous solutions.

The formulations herein may also contain more than one active compound as necessary for the particular indication being treated, preferably those with complementary activities that do not adversely affect each other. For example, it may be desirable to provide antibodies or antigen binding fragments thereof with other specificities. Alternatively, or in addition, the composition may comprise a cytotoxic agent, cytokine, growth inhibitory agent and/or small molecule antagonist. Such molecules are suitably present in combination in amounts that are effective for the purpose intended.

In some embodiments, two mezagitamab drug product formulations have been developed, referred to Process A and Process B as disclosed herein.

In one embodiment, the Process A mezagitamab drug product is a clear-to-opalescent, colorless solution containing TAK-079 (20 mg/mL) aqueous solution of arginine hydrochloride, anhydrous citric acid, sodium citrate, polysorbate 80, and water for injection at approximately pH 6.5. The Process A placebo is a clear, colorless solution containing an aqueous solution of arginine hydrochloride, anhydrous citric acid, sodium citrate, polysorbate 80, and water for injection at approximately pH 6.5. The Process A mezagitamab drug product and placebo are supplied in aseptically filled, single-use, clear, type I borosilicate glass vials with fluoropolymer coated butyl rubber stoppers and aluminum crimp seals with flip-off caps.

In another embodiment, the Process B mezagitamab drug product is made in 2 strengths, 5 mg/mL or 100 mg/mL. Each strength is a clear-to-opalescent, colorless-to-brownish-yellow solution containing mezagitamab in an aqueous solution of histidine, histidine hydrochloride monohydrate, sucrose, polysorbate 20, and water for injection at approximately pH 5.9. The Process B placebo is a clear, colorless solution containing an aqueous solution of histidine, histidine hydrochloride monohydrate, sucrose, polysorbate 20, and water for injection at approximately pH 5.9. The Process B mezagitamab drug product and placebo are supplied in aseptically filled, single use, clear, type I, borosilicate glass vials with fluoropolymer coated butyl rubber stoppers and aluminum crimp seals with flip-off caps.

### Subcutaneous Administration

The anti-CD38 antibodies described herein, such as TAK-079, can be administered at sufficiently dosages that are therapeutically effective, thereby allowing for subcutaneous administration. Subcutaneous administration is a minimally invasive mode of administration and is considered the most versatile and therefore desirable mode of administration that can be used for short-term and long-term therapies. In some embodiments, subcutaneous administration can be performed by injection. In some embodiments, the site of the injection or device can be rotated when multiple injections or devices are needed.

Accordingly, subcutaneous formulations are much easier for a patient to self-administer, especially since the formulation may have to be taken regularly during the patient's entire life. Furthermore, the ease and speed of subcutaneous delivery allows increased patient compliance and quicker access to medication when needed. Thus, the subcutaneous formulations of the anti-CD38 antibodies provided herein provide a substantial benefit over the prior art and solve certain unmet needs.

In some embodiments, the antibodies of the invention are administered to a subject in accordance with known methods via a subcutaneous route. In some embodiments, antibodies of the present invention can be administered by subcutaneous injection. In specific embodiments, the subcutaneous formulation is subcutaneously injected into the same site of a patient (e.g., administered to the upper arm, anterior surface of the thigh, lower portion of the abdomen, or upper back) for repeat or continuous injections. In other embodiments, the subcutaneous formulation is subcutaneously injected into a different or rotating site of a patient. Single or multiple administrations of the formulations may be employed.

In some embodiments, the subcutaneous unit dosage forms described herein can be used for the treatment of SLE. In some embodiments, the subcutaneous unit dosage forms described herein can be used for the treatment of moderate or severe SLE. In some embodiments, the subcutaneous unit dosage forms described herein can be used for the treatment of moderate SLE. In some embodiments, the subcutaneous unit dosage forms described herein can be used for the treatment of severe SLE.

In some embodiments, the antibodies or antigen binding fragments thereof of the invention lead to depletion of plasmablasts, plasma cells, NK cells, B cells and/or T cells after subcutaneous administration to a subject. In some embodiments, the antibodies or antigen binding fragments thereof of the invention lead to depletion of plasmablasts. In some embodiments, the antibodies or antigen binding fragments thereof of the invention lead to depletion of plasma cells. In some embodiments, the antibodies or antigen binding fragments thereof of the invention allow for increased depletion of NK cells as compared to the depletion of B cells or T cells. In some embodiments, the antibodies or antigen binding fragments thereof of the invention allow for increased depletion of NK cells as compared to B cells, as well as increased depletion of NK cells as compared to T cells. In some embodiments, the antibodies or antigen binding fragments thereof of the invention allow for increased depletion of NK cells as compared to B cells, as well as increased depletion of B cells as compared to T cells. In some embodiments, the antibodies or antigen binding fragments thereof of the invention allow for increased depletion of NK cells as compared to B cells and increased depletion of B cells as compared to T cells. Suitably, the antibodies or antigen binding fragments thereof of the invention may allow for increased depletion of CD38⁺ cells as compared to CD38⁻ cells.

In some embodiments, the antibodies or antigen binding fragments thereof of the invention lead to a decrease in the level of immunoglobulin(s) after subcutaneous administration to a subject. In some embodiments, the immunoglobulin is IgA, IgG and/or IgM. In some embodiments, the immunoglobulin is IgA. In some embodiments, the immunoglobulin is IgG. In some embodiments, the immunoglobulin is IgM.

In some embodiments, the antibodies or antigen binding fragments thereof of the invention lead to a decrease in one or more autoantibodies after subcutaneous administration to a subject. In some embodiments, the one or more autoantibodies is selected from the group consisting of anti-dsDNA, anti-SmDp, beta-2 glycoprotein 1 IgM, ribonucleoprotein-70, Sjogrens SS-A and Sjogrens SS-B.

In certain embodiments, the bioavailability of the anti-CD38 antibodies described herein after subcutaneous administration is between at least 50% and at least 80% as compared to intravenous administration normalized for the same dose. In certain embodiments, the bioavailability of the anti-CD38 antibodies described herein after subcutaneous administration is between at least 60% and at least 80% as compared to intravenous administration normalized for the same dose. In certain embodiments, the bioavailability of the anti-CD38 antibodies described herein after subcutaneous administration is between at least 50% and 70% as compared to intravenous administration normalized for the same dose. In certain embodiments, the bioavailability of the anti-CD38 antibodies described herein after subcutaneous administration is between at least 55% and 65% as compared to intravenous administration normalized for the same dose. In certain embodiments, the bioavailability of the anti-CD38 antibodies described herein after subcutaneous administration is between at least 55% and 70% as compared to intravenous administration normalized for the same dose.

In certain embodiments, the bioavailability of the anti-CD38 antibodies described herein after subcutaneous administration is at least 40%, at least 45%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, or at least 85% as compared to intravenous administration normalized for the same dose. Suitably the bioavailability may be at least 50% as compared to intravenous administration normalized for the same dose. Suitably the bioavailability may be at least 60% as compared to intravenous administration normalized for the same dose. Suitably the bioavailability may be at least 70% as compared to intravenous administration normalized for the same dose. Suitably the bioavailability may be at least 80% as compared to intravenous administration normalized for the same dose. Suitably the bioavailability may be at least 90% as compared to intravenous administration normalized for the same dose.

In some embodiments, the present disclosure provides a method wherein the bioavailability of the antibodies of the invention after subcutaneous administration is 50%-80% as compared to intravenous administration normalized for the same dose.

In some embodiments, the present disclosure provides a method wherein the bioavailability of the antibodies of the invention after subcutaneous administration is at least 50% as compared to intravenous administration normalized for the same dose.

In some embodiments, the present disclosure provides a method wherein the bioavailability of the antibodies of the invention after subcutaneous administration is at least 55% as compared to intravenous administration normalized for the same dose.

In some embodiments, the present disclosure provides a method wherein the bioavailability of the antibodies of the invention after subcutaneous administration is at least 60% as compared to intravenous administration normalized for the same dose.

In some embodiments, the present disclosure provides a method wherein the bioavailability of the antibodies of the invention after subcutaneous administration is at least 65% as compared to intravenous administration normalized for the same dose.

In some embodiments, the present disclosure provides a method wherein the bioavailability of the antibodies of the invention after subcutaneous administration is at least 70% as compared to intravenous administration normalized for the same dose.

In some embodiments, the present disclosure provides a method wherein the bioavailability of the antibodies of the invention after subcutaneous administration is at least 75% as compared to intravenous administration normalized for the same dose.

In some embodiments, the present disclosure provides a method wherein the bioavailability of the antibodies of the invention after subcutaneous administration is at least 80% as compared to intravenous administration normalized for the same dose.

In certain embodiments, the anti-CD38 antibodies or antigen binding fragments thereof as described herein are subcutaneously administered in a single bolus injection. In certain embodiments, the anti-CD38 antibodies or antigen binding fragments thereof as described herein are subcutaneously administered monthly. In certain embodiments, the anti-CD38 antibodies or antigen binding fragments thereof as described herein are subcutaneously administered every two weeks. In certain embodiments, the anti-CD38 antibodies or antigen binding fragments thereof as described herein are subcutaneously administered weekly. In certain embodiments, the anti-CD38 antibodies or antigen binding fragments thereof as described herein are subcutaneously administered twice a week. In certain embodiments, the anti-CD38 antibodies or antigen binding fragments thereof as described herein are subcutaneously administered daily. In certain embodiments, the anti-CD38 antibodies or antigen binding fragments thereof as described herein are subcutaneously administered every 12 hours. In certain embodiments, the anti-CD38 antibodies or antigen binding fragments thereof as described herein are subcutaneously administered every 8 hours. In certain embodiments, the anti-CD38 antibodies or antigen binding fragments thereof as described herein are subcutaneously administered every 6 hours. In certain embodiments, the anti-CD38 antibodies or antigen binding fragments thereof as described herein are subcutaneously administered every 4 hours. In certain embodiments, the anti-CD38 antibodies or antigen binding fragments thereof as described herein are subcutaneously administered every 2 hours. In certain embodiments, the anti-CD38 antibodies or antigen binding fragments thereof as described herein are subcutaneously administered every hour. In some embodiments, the antibodies or antigen binding fragments thereof as disclosed herein is subcutaneously administered once every 3 weeks for 12 weeks.

In some embodiments, the anti-CD38 antibodies or antigen binding fragments thereof as disclosed herein are subcutaneously administered at a dosage of from about 40 milligrams to about 140 milligrams. In some embodiments, the anti-CD38 antibodies or antigen binding fragments thereof as disclosed herein are subcutaneously administered at a dosage selected from the group consisting of about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, and about 140 mg. In some embodiments, the antibody or antigen binding fragment thereof is administered in a dosage of about 45 mg, about 90 mg or about 135 mg. In some embodiments, the antibody or antigen binding fragment thereof is administered in a dosage of about 45 mg. In some embodiments, the antibody or antigen binding fragment thereof is administered in a dosage of about 90 mg. In some embodiments, the antibody or antigen binding fragment thereof is administered in a dosage of about 135 mg.

### Unit Dosage Forms

In some embodiments, the therapeutic anti-CD38 antibodies or antigen binding fragments thereof are formulated as part of a unit dosage form. In some embodiments, the anti-CD38 antibody or antigen binding fragment thereof comprises an HC comprising the following CDR amino acid sequences: GFTFDDYG (SEQ ID NO:3; HCDR1 TAK-079), ISWNGGKT (SEQ ID NO:4; HCDR2 TAK-079), and ARGSLFHDSSGFYFGH (SEQ ID NO:5; HCDR3 TAK-079) or variants of those sequences having up to three amino acid changes. In some embodiments, the antibody or antigen binding fragment thereof comprises an LC comprising the following CDR amino acid sequences: SSNIGDNY (SEQ ID NO:6; LCDR1 TAK-079), RDS (SEQ ID NO:7; LCDR2 TAK-079), and QSYDSSLSGS (SEQ ID NO:8; LCDR3 TAK-079) or variants of those sequences having up to three amino acid changes. In some embodiments, the antibody comprises an HC comprising the following CDR amino acid sequences: GFTFDDYG (SEQ ID NO:3; HCDR1 TAK-079), ISWNGGKT (SEQ ID NO:4; HCDR2 TAK-079), ARGSLFHDSSGFYFGH (SEQ TD NO:5; HCDR3 TAK-079) or variants of those sequences having up to three amino acid changes and an LC comprising the following CDR amino acid sequences: SSNIGDNY (SEQ ID NO:6; LCDR1 TAK-079), RDS (SEQ ID NO:7; LCDR2 TAK-079), and QSYDSSLSGS (SEQ ID NO:8; LCDR3 TAK-079) or variants of those sequences having up to three amino acid changes. In some embodiments, the antibody comprises an HC comprising the following CDR amino acid sequences: GFTFDDYG (SEQ ID NO:3; HCDR1 TAK-079), ISWNGGKT (SEQ ID NO:4; HCDR2 TAK-079), and ARGSLFHDSSGFYFGH (SEQ ID NO:5; HCDR3 TAK-079). In some embodiments, the antibody comprises an LC comprising the following CDR amino acid sequences: SSNIGDNY (SEQ ID NO:6; LCDR1 TAK-079), RDS (SEQ ID NO:7; LCDR2 TAK-079), and QSYDSSLSGS (SEQ ID NO:8; LCDR3 TAK-079). In some embodiments, the antibody comprises an HC comprising the following CDR amino acid sequences: GFTFDDYG (SEQ ID NO:3; HCDR1 TAK-079), ISWNGGKT (SEQ ID NO:4; HCDR2 TAK-079), ARGSLFHDSSGFYFGH (SEQ ID NO:5; HCDR3 TAK-079) and an LC comprising the following CDR amino acid sequences: SSNIGDNY (SEQ ID NO:6; LCDR1 TAK-079), RDS (SEQ ID NO:7; LCDR2 TAK-079), and QSYDSSLSGS (SEQ ID NO:8; LCDR3 TAK-079). In some embodiments, the antibody or antigen binding fragment thereof comprises an HC comprising an amino acid sequence having at least 80% sequence identity to SEQ ID NO:9.

Suitably, the HC may comprise the following CDR amino acid sequences: GFTFDDYG (SEQ ID NO:3; HCDR1 TAK-079), ISWNGGKT (SEQ ID NO:4; HCDR2 TAK-079), and ARGSLFHDSSGFYFGH (SEQ ID NO:5; HCDR3 TAK-079) and the remainder of the HC may have at least 80% sequence identity to SEQ ID NO 9. In some embodiments, the antibody comprises an HC comprising the VH region amino acid sequence of SEQ ID NO:9.

(SEQ ID NO: 9)
EVQLLESGGGLVQPGGSLRLSCAASGFTFDDYGMSWVRQAPGKGLEWVSD
ISWNGGKTHYVDSVKGQFTISRDNSKNTLYLQMNSLRAEDTAVYYCARGS
LFHDSSGFYFGHWGQGTLVTVSSASTKGPSVFPLA.

In some embodiments, the antibody comprises an LC comprising an amino acid sequence having at least 80% sequence identity to SEQ ID NO:10. Suitably, the LC may comprise the following CDR sequences: SSNIGDNY (SEQ ID NO:6; LCDR1 TAK-079), RDS (SEQ ID NO:7; LCDR2 TAK-079), and QSYDSSLSGS (SEQ ID NO:8; LCDR3 TAK-079) and the remainder of the LC may have at least 80% sequence identity to SEQ ID NO: 10. In some embodiments, the antibody comprises an LC comprising the VL region amino acid sequence of SEQ ID NO:10.

(SEQ ID NO: 10)
QSVLTQPPSASGTPGQRVTISCSGSSSNIGDNYVSWYQQLPGTAPKLLI
YRDSQRPSGVPDRFSGSKSGTSASLAISGLRSEDEADYYCQSYDSSLSG
SVFGGGTKLTVLGQPKANPTVTLFPPSSEEL.

In some embodiments, the antibody comprises an HC comprising the VH region amino acid sequence of SEQ ID NO:9 or a variant thereof as described herein and an LC comprising the VL region amino acid sequence of SEQ ID NO:10 or a variant thereof as described herein.

As will be appreciated by those in the art, the VH region and VL region can be joined to human IgG constant domain sequences, generally IgG1, IgG2 or IgG4. In some embodiments, the antibody comprises an HC having amino acid sequence with at least 80% sequence identity to SEQ ID NO:11. Suitably, the HC may comprise the CDR sequences as defined by SEQ ID NO: 3, SEQ ID NO: 4 and SEQ ID NO: 5 and the remainder of the HC may have at least 80% sequence identity to SEQ ID NO 11. In some embodiments, the antibody comprises the HC amino acid sequence of SEQ ID NO:11.

(SEQ ID NO: 11)
EVQLLESGGGLVQPGGSLRLSCAASGFTFDDYGMSWVRQAPGKGLEWVS
DISWNGGKTHYVDSVKGQFTISRDNSKNTLYLQMNSLRAEDTAVYYCAR
GSLFHDSSGFYFGHWGQGTLVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKRVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNH
YTQKSLSLSPGK.

In some embodiments, the antibody comprises an LC having amino acid sequence with at least 80% sequence identity to SEQ ID NO:12. Suitably, the LC may comprise the CDR sequences as defined by SEQ ID NO: 6, SEQ ID NO: 7 and SEQ ID NO: 8 and the remainder of the LC may have at least 80% sequence identity to SEQ ID NO 12. In some embodiments, the antibody comprises the LC amino acid sequence of SEQ ID NO:12.

(SEQ ID NO: 12)
QSVLTQPPSASGTPGQRVTISCSGSSSNIGDNYVSWYQQLPGTAPKLLI
YRDSQRPSGVPDRFSGSKSGTSASLAISGLRSEDEADYYCQSYDSSLSG
SVFGGGTKLTVLGQPKANPTVTLFPPSSEELQANKATLVCLISDFYPGA
VTVAWKADGSPVKAGVETTKPSKQSNNKYAASSYLSLTPEQWKSHRSYS
CQVTHEGSTVEKTVAPTECS.

In some embodiments, the antibody comprises the HC amino acid sequence of SEQ ID NO:11 or a variant thereof as described herein and the LC amino acid sequence of SEQ ID NO:12 or a variant thereof as described herein.

In some embodiments, the formulation comprising the anti-CD38 antibody is a unit dosage form. In some embodiments, the unit dosage form comprises an amount sufficient to administer a dosage of about 40 mg to about 140 mg. In some embodiments, the unit dosage form comprises an amount sufficient to administer a dosage of about 45 mg to about 135 mg. In some embodiments, the unit dosage form comprises an amount sufficient to administer a dosage of about 45 mg to about 140 mg. In some embodiments, the unit dosage form comprises an amount sufficient to administer a dosage of about 40 mg to about 135 mg. In some embodiments, the unit dosage form comprises an amount sufficient to administer a dosage selected from the group consisting of about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, and about 140 mg. In some embodiments, the antibody or antigen binding fragment thereof is administered in a dosage of about 45 mg, about 90 mg or about 135 mg.

In some embodiments, the unit dosage form comprises an amount sufficient to administer a dosage of about 40 mg. In some embodiments, the unit dosage form comprises an amount sufficient to administer a dosage of about 45 mg. In some embodiments, the unit dosage form comprises an amount sufficient to administer a dosage of about 50 mg. In some embodiments, the unit dosage form comprises an amount sufficient to administer a dosage of about 55 mg. In some embodiments, the unit dosage form comprises an amount sufficient to administer a dosage of about 60 mg. In some embodiments, the unit dosage form comprises an amount sufficient to administer a dosage of about 65 mg. In some embodiments, the unit dosage form comprises an amount sufficient to administer a dosage of about 70 mg. In some embodiments, the unit dosage form comprises an amount sufficient to administer a dosage of about 75 mg. In some embodiments, the unit dosage form comprises an amount sufficient to administer a dosage of about 80 mg. In some embodiments, the unit dosage form comprises an amount sufficient to administer a dosage of about 85 mg. In some embodiments, the unit dosage form comprises an amount sufficient to administer a dosage of about 90 mg. In some embodiments, the unit dosage form comprises an amount sufficient to administer a dosage of about 95 mg. In some embodiments, the unit dosage form comprises an amount sufficient to administer a dosage of about 100 mg. In some embodiments, the unit dosage form comprises an amount sufficient to administer a dosage of about 105 mg. In some embodiments, the unit dosage form comprises an amount sufficient to administer a dosage of about 110 mg. In some embodiments, the unit dosage form comprises an amount sufficient to administer a dosage of about 115 mg. In some embodiments, the unit dosage form comprises an amount sufficient to administer a dosage of about 120 mg. In some embodiments, the unit dosage form comprises an amount sufficient to administer a dosage of about 125 mg. In some embodiments, the unit dosage form comprises an amount sufficient to administer a dosage of about 130 mg. In some embodiments, the unit dosage form comprises an amount sufficient to administer a dosage of about 135 mg. In some embodiments, the unit dosage form comprises an amount sufficient to administer a dosage of about 140 mg.

In some embodiments, the anti-CD38 antibody unit dosage forms provided herein may further comprise one or more pharmaceutically acceptable excipients, carriers, and/or diluents. In some embodiments, the anti-CD38 antibody is provided as a pharmaceutical composition which comprises a unit dosage form according to the present invention. Suitably, the pharmaceutical composition may further comprise one or more pharmaceutically acceptable excipients, carriers, and/or diluents.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Compositions may be formulated in dosage unit form for ease of administration and uniformity of dosage. Dosage unit forms as used herein can, in some embodiments, refer to physically discrete units suited as unitary dosages for the subjects to be treated, each unit containing a predetermined quantity of active compound calculated to produce the desired therapeutic effect in association with the required pharmaceutical carrier.

The specification for the dosage unit forms of the present invention is dictated by and are directly dependent on (a) the unique characteristics of the active compound and the particular therapeutic effect to be achieved, and (b) the limitations inherent in the art of compounding such an active compound for the treatment of an individual.

The efficient dosages and the dosage regimens for the anti-CD38 antibodies or antigen binding fragments thereof used in the present invention depend on the severity of the disease or condition to be treated and may be determined by persons skilled in the art.

In some embodiments, the anti-CD38 antibody or antigen binding fragments thereof is administered by subcutaneous administration once every week, once every two weeks, once every three weeks or once every four weeks in a dosage of about 40 mg to about 140 mg. In some embodiments, the anti-CD38 antibody or antigen binding fragments thereof is administered by subcutaneous administration once every week in a dosage of about 40 mg to about 140 mg.

In some embodiments, the anti-CD38 antibody or antigen binding fragments thereof is administered by subcutaneous administration once every two weeks in a dosage of about 40 mg to about 140 mg. In some embodiments, the anti-CD38 antibody or antigen binding fragments thereof is administered by subcutaneous administration once every three weeks in a dosage of about 40 mg to about 140 mg. In some embodiments, the anti-CD38 antibody or antigen binding fragments thereof is administered by subcutaneous administration once every four weeks in a dosage of about 40 mg to about 140 mg.

Suitably, the dosage every 3 weeks may be about 40 mg. Suitably, the dosage every 3 weeks may be about 45 mg. Suitably, the dosage every 3 weeks may be about 50 mg. Suitably, the dosage every 3 weeks may be about 55 mg. Suitably, the dosage every 3 weeks may be about 60 mg. Suitably, the dosage every 3 weeks may be about 65 mg. Suitably, the dosage every 3 weeks may be about 70 mg. Suitably, the dosage every 3 weeks may be about 75 mg. Suitably, the dosage every 3 weeks may be about 80 mg. Suitably, the dosage every 3 weeks may be about 85 mg. Suitably, the dosage every 3 weeks may be about 90 mg. Suitably, the dosage every 3 weeks may be about 95 mg. Suitably, the dosage every 3 weeks may be about 100 mg. Suitably, the dosage every 3 weeks may be about 105 mg. Suitably, the dosage every 3 weeks may be about 110 mg. Suitably, the dosage every 3 weeks may be about 115 mg. Suitably, the dosage every 3 weeks may be about 120 mg. Suitably, the dosage every 3 weeks may be about 125 mg. Suitably, the dosage every 3 weeks may be about 130 mg. Suitably, the dosage every 3 weeks may be about 135 mg. Suitably, the dosage every 3 weeks may be about 140 mg. Such administration as disclosed herein may be repeated, e.g., 3 to 5 times. In some embodiments, such administration as disclosed herein may be repeated 3 times, i.e., every 3 weeks for a total of 9 weeks. In some embodiments, such administration as disclosed herein may be repeated 4 times, i.e., every 3 weeks for a total of 12 weeks. In some embodiments, such administration as disclosed herein may be repeated 5 times, i.e., every 3 weeks for a total of 15 weeks.

In one embodiment, the anti-CD38 antibody or antigen binding fragment thereof is administered in a 3-week dosage of about 40 mg to about 140 mg. Suitably, the 3-week dosage may be about 45 mg to about 135 mg. Suitably, the 3-week dosage may be about 45 mg. Suitably, the 3-week dosage may be about 90 mg. Suitably, the 3-week dosage may be about 135 mg. The dosage may be determined or adjusted by measuring the amount of compound of the present invention in the blood upon administration, for instance, by taking a biological sample and using anti-idiotypic antibodies that target the antigen binding region of the anti-CD38 antibody.

In one embodiment, the therapeutic antibody is formulated at about 5 mg/ml concentration. In another embodiment, the therapeutic antibody is formulated at about 20 mg/ml concentration. In another embodiment, the therapeutic antibody is formulated at about 50 mg/ml concentration. In some embodiments, 0.8 mL, 0.9 mL, 1.8 mL, 2.7 mL or 2.8 mL volume is injected in the thigh, abdomen, or arm. In another embodiment, the therapeutic antibody is formulated at about 75 mg/ml concentration. In some embodiments, 0.53 mL, 0.6 mL, 1.2 mL, 1.8 mL or 1.87 mL volume is injected in the thigh, abdomen, or arm. In another embodiment, the therapeutic antibody is formulated at about 90 mg/ml concentration. In some embodiments, 0.44 mL, 0.5 mL, 1.0 mL, 1.5 mL or 1.56 mL volume is injected in the thigh, abdomen, or arm. In another embodiment, the therapeutic antibody is formulated at about 100 mg/ml concentration. In some embodiments, 0.4 mL, 0.45 mL, 0.9 mL, 1.35 mL or 1.4 mL volume is injected in the thigh, abdomen, or arm. In some embodiments, the dose is administered over a 1-, 2-, 4-, 6-, 8-, or 10-hour period of time. In some embodiments, the doses are administered every week. In some embodiments, the doses are administered every 2 weeks. In some embodiments, the doses are administered every 3 weeks. In some embodiments, the doses are administered every 4 weeks.

In some embodiments, the invention provides a unit dosage form comprising an isolated antibody or antigen binding fragment thereof that comprises a VH region comprising a CDR1 having the amino acid sequence of SEQ ID NO:3, a CDR2 having the amino acid sequence of SEQ ID NO:4, and a CDR3 having the amino acid sequence of SEQ ID NO:5; and a VL region comprising a CDR1 having the amino acid sequence of SEQ ID NO:6, a CDR2 having the amino acid sequence of SEQ ID NO:7, and a CDR3 having the amino acid sequence of SEQ ID NO:8; wherein the isolated antibody or antigen binding fragment thereof binds to human CD38 (SEQ ID NO:1), and the unit dosage form is formulated for subcutaneous administration of the antibody or antigen binding fragment thereof at a dosage of from 40 milligrams to 140 milligrams in the treatment of moderate or severe SLE.

In some embodiments, the invention provides the unit dosage form as disclosed herein, wherein the unit dosage form is formulated for subcutaneous administration of the antibody or antigen binding fragment thereof in the treatment of severe SLE.

In some embodiments, the invention provides the unit dosage form as disclosed herein, wherein the antibody or antigen binding fragment thereof further comprises one or more engineered glycoforms. In some embodiments, the engineered glycoform comprises glycosylation of one or more polypeptides, and the glycosylation is N-linked glycosylation or 0-linked glycosylation. In some embodiments, the glycosylation is N-linked glycosylation. In some embodiments, the glycosylation is O-linked glycosylation.

In some embodiments, the invention provides the unit dosage form as disclosed herein, wherein the VH region of the antibody or antigen binding fragment thereof comprises an amino acid sequence having an identity of at least 90% to SEQ ID NO:9, and/or the VL region of the antibody or antigen binding fragment thereof comprises an amino acid sequence having an identity of at least 90% to SEQ ID NO:10.

In some embodiments, the invention provides the unit dosage form as disclosed herein, wherein the VH region comprises an amino acid sequence having an identity of at least 95% to SEQ ID NO:9.

In some embodiments, the invention provides the unit dosage form as disclosed herein, wherein the VL region comprises an amino acid sequence having an identity of at least 95% to SEQ ID NO:10.

In some embodiments, the invention provides the unit dosage form as disclosed herein, wherein the VH region comprises an amino acid sequence having an identity of at least 99% to SEQ ID NO:9.

In some embodiments, the invention provides the unit dosage form as disclosed herein, wherein the VL region comprises an amino acid sequence having an identity of at least 99% to SEQ ID NO:10.

In some embodiments, the invention provides the unit dosage form as disclosed herein, wherein the HC of the antibody or antigen binding fragment thereof comprises an amino acid sequence having an identity of at least 95% to SEQ ID NO:11.

In some embodiments, the invention provides the unit dosage form as disclosed herein, wherein the LC of the antibody or antigen binding fragment thereof comprises an amino acid sequence having an identity of at least 95% to SEQ ID NO:12.

In some embodiments, the invention provides the unit dosage form as disclosed herein, wherein the isolated antibody or antigen binding fragment thereof interacts with at least K121, F135, Q139, D141, E239, W241, C275, K276, F284, P291 and E292 of SEQ ID NO:1 and SEQ ID NO:2, based on human sequence numbering.

In some embodiments, the invention provides the unit dosage form as disclosed herein, wherein the isolated antibody or antigen binding fragment thereof binds to human CD38 (SEQ ID NO:1) with a KD of 10⁻⁸ M or a greater affinity, and wherein the affinity is measured by a standard Biacore assay.

In some embodiments, the invention provides the unit dosage form as disclosed herein, wherein the VH region comprises SEQ ID NO:9 and the VL region comprises SEQ ID NO:10.

In some embodiments, the invention provides the unit dosage form as disclosed herein, wherein the isolated antibody or antigen binding fragment thereof comprises an HC as set forth in SEQ ID NO:11 and a LC as set forth in SEQ ID NO: 12.

In some embodiments, the invention provides the unit dosage form as disclosed herein, wherein the isolated antibody or antigen binding fragment thereof further comprises an Fc domain. In some embodiments, the Fc domain is a human Fc domain. In some embodiments, the Fc domain is a variant Fc domain.

In some embodiments, the invention provides the unit dosage form as disclosed herein, wherein the isolated antibody or antigen binding fragment is a human IgG antibody. In some embodiments, the human IgG antibody is a human IgG1 antibody.

In some embodiments, the invention provides the unit dosage form as disclosed herein further comprising background SLE medication(s).

In some embodiments, the invention provides the unit dosage form as disclosed herein, wherein the isolated antibody or antigen binding fragment thereof is used in combination with one or more background SLE medications. In some embodiments, the background SLE medication(s) is selected from the group consisting of immunosuppressants, steroids and immunoglobulin. In some embodiments, the background SLE medication(s) is selected from the group consisting of hydroxychloroquine, hydroxychloroquine sulfate, prednisone, methylprednisolone, gabapentin, mycophenolate mofetil, and/or mycophenolic acid. In some embodiments, the unit dosage form as disclosed herein comprises the one or more background SLE medications.

In some embodiments, the invention provides the unit dosage form as disclosed herein, wherein the antibody or antigen binding fragment thereof is administered in a dosage selected from the group consisting of 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, and 140 mg. In some embodiments, the antibody or antigen binding fragment thereof is administered in a dosage of 40 mg. In some embodiments, the antibody or antigen binding fragment thereof is administered in a dosage of 45 mg. In some embodiments, the antibody or antigen binding fragment thereof is administered in a dosage of 50 mg. In some embodiments, the antibody or antigen binding fragment thereof is administered in a dosage of 55 mg. In some embodiments, the antibody or antigen binding fragment thereof is administered in a dosage of 60 mg. In some embodiments, the antibody or antigen binding fragment thereof is administered in a dosage of 65 mg. In some embodiments, the antibody or antigen binding fragment thereof is administered in a dosage of 70 mg. In some embodiments, the antibody or antigen binding fragment thereof is administered in a dosage of 75 mg. In some embodiments, the antibody or antigen binding fragment thereof is administered in a dosage of 80 mg. In some embodiments, the antibody or antigen binding fragment thereof is administered in a dosage of 85 mg. In some embodiments, the antibody or antigen binding fragment thereof is administered in a dosage of 90 mg. In some embodiments, the antibody or antigen binding fragment thereof is administered in a dosage of 95 mg. In some embodiments, the antibody or antigen binding fragment thereof is administered in a dosage of 100 mg. In some embodiments, the antibody or antigen binding fragment thereof is administered in a dosage of 105 mg. In some embodiments, the antibody or antigen binding fragment thereof is administered in a dosage of 110 mg. In some embodiments, the antibody or antigen binding fragment thereof is administered in a dosage of 115 mg. In some embodiments, the antibody or antigen binding fragment thereof is administered in a dosage of 120 mg. In some embodiments, the antibody or antigen binding fragment thereof is administered in a dosage of 125 mg. In some embodiments, the antibody or antigen binding fragment thereof is administered in a dosage of 130 mg. In some embodiments, the antibody or antigen binding fragment thereof is administered in a dosage of 135 mg. In some embodiments, the antibody or antigen binding fragment thereof is administered in a dosage of 140 mg.

In some embodiments, the invention provides the unit dosage form as disclosed herein, wherein the dosage is a dosage administered once every week, once every two weeks, once every three weeks or once every four weeks.

In some embodiments, the invention provides the unit dosage form as disclosed herein further comprising at least one pharmaceutically acceptable carrier, excipient or stabilizer.

In some embodiments, the invention provides the unit dosage form as disclosed herein, wherein administering the antibody or antigen binding fragment thereof results in less than 10% incidence of grade 3 or 4 of one or more treatment-related adverse events (TRAEs) or treatment-emergent adverse events (TEAEs). In some embodiments, the TRAEs or TEAEs are selected from the group consisting of gastrointestinal disorders, nausea, infestations, pyrexia, Herpes Zoster, urinary tract infection, skin and cutaneous tissue disorders, headache, fever, chills/rigors, vomiting, diarrhea, arthralgia, myalgia, hypotension, respiratory, thoracic and mediastinal disorders, thrombocytopenia, leukopenia, lymphopenia, cardiac disorders, palpitations, and dyspnea. In some embodiments, the administration of the antibody or antigen binding fragment thereof results in one or more TRAEs or TEAEs having a maximum intensity of Common Terminology Criteria for Adverse Events (CTCAE) Grade 1 or Grade 2.

In some embodiments, the invention provides the unit dosage form as disclosed herein, wherein the isolated antibody or antigen binding fragment thereof comprises an HC as set forth in SEQ ID NO:11 and an LC as set forth in SEQ ID NO:12; and wherein the antibody or antigen binding fragment thereof is subcutaneously administered once every 3 weeks for 12 weeks. In some embodiments, the invention provides the unit dosage form as disclosed herein, wherein the isolated antibody or antigen binding fragment thereof further comprises one or more engineered glycoforms, wherein the engineered glycoform comprises glycosylation of one or more polypeptides and the glycosylation is N-linked glycosylation.

### Treatment Modalities

In the methods of the invention, therapy is used to provide a positive therapeutic response with respect to a disease or condition. The term “positive therapeutic response” refers to an improvement in a disease or condition, and/or an improvement in the symptoms associated with the disease or condition.

Positive therapeutic responses in any given disease or condition can be determined by standardized response criteria specific to that disease or condition. In addition to the positive therapeutic responses, the subject undergoing therapy may experience the beneficial effect of an improvement in the symptoms associated with the disease.

Measurements of efficacy in treating SLE can be assessed based on SLE disease activity scales in accordance with the SOE study activity tables (Table 4 and Table 5, Example 1). The SLE disease assessments are based on assessment tools including but not limited to SLEDAI-2K disease assessment tool, Cutaneous Lupus Erythematosus Disease Area and Severity Index, 44-Joint Assessment, Physician's Global Assessment of Disease (Visual Analog Scale) as disclosed in Example 1.

Treatment according to the present invention includes a “therapeutically effective amount” of the medicaments used. The terms “therapeutically effective amount” and “therapeutically effective dosage” refer to an amount of a therapy that is sufficient to reduce or ameliorate the severity and/or duration of a disorder or one or more symptoms thereof, prevent the advancement of a disorder; cause regression of a disorder; prevent the recurrence, development, onset, or progression of one or more symptoms associated with a disorder; or enhance or improve the prophylactic or therapeutic effect(s) of another therapy (e.g., prophylactic or therapeutic agent), at dosages and for periods of time necessary to achieve a desired therapeutic result. A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

### Anti-CD38 Antibody Kits

In another aspect of the invention, kits are provided for the treatment of SLE. In some embodiments, kits are provided for the treatment of moderate or severe SLE. In some embodiments, kits are provided for the treatment of severe SLE. In one embodiment, the kit comprises a dose of an anti-CD38 antibody described herein, such as TAK-079. In one embodiment, the kit comprises a dose of an anti-CD38 antibody described herein, such as Mezagitamab. In some embodiments, the kits provided herein may contain one or more doses of a liquid or lyophilized formulation as provided herein. When the kits comprise a lyophilized formulation of an anti-CD38 antibody described herein such as TAK-079, generally the kits will also contain a suitable liquid for reconstitution of the liquid formulation, for example, sterile water or a pharmaceutically acceptable buffer. In some embodiments, the kits may comprise an anti-CD38 antibody formulation described herein prepackaged in a syringe for subcutaneous administration by a health care professional or for home use.

In certain embodiments, the kit will be for a single administration or dose of an anti-CD38 antibody described herein such as TAK-079. In other embodiments, the kit may contain multiple doses of an anti-CD38 antibody described herein such as TAK-079 for subcutaneous administration. In one embodiment, the kit may comprise an anti-CD38 antibody formulation described herein prepackaged in a syringe for subcutaneous administration by a health care professional or for home use.

### Articles Of Manufacture

In other embodiments, an article of manufacture containing materials useful for the treatment of the disorders described above is provided. The article of manufacture comprises a container and a label. Suitable containers include, for example, bottles, vials, syringes, and test tubes. The containers may be formed from a variety of materials such as glass or plastic. The container holds a composition which is effective for treating the condition and may have a sterile access port (for example, the container may be an intravenous solution bag or a vial having a stopper pierceable by a hypodermic injection needle). The active agent in the composition is the antibody. The label on, or associated with, the container indicates that the composition is used for treating the condition of choice. The article of manufacture may further comprise a second container comprising a pharmaceutically acceptable buffer, such as phosphate-buffered saline, Ringer's solution or dextrose solution. It may further include other materials desirable from a commercial and user standpoint, including other buffers, diluents, filters, needles, syringes, and package inserts with instructions for use.

### EXAMPLES

### Example 1: a Phase 1B Study to Evaluate the Safety, Pharmacokinetics, and Pharmacodynamics of Tak-079 in Combination with Standard Background Therapy in Patients with Moderate to Severe Systemic Lupus Erythematosus Study Objectives and Investigational Plan

This phase 1b double-blind, placebo-controlled, multicenter study evaluated the safety, pharmacokinetics, and pharmacodynamics of mezagitamab in a study population receiving standard principal investigator-directed background therapy for moderate to severe SLE.

The primary objective of this study was to evaluate the safety and tolerability of TAK-079 in comparison with matching placebo, administered once every 3 weeks over a 12-week dosing period in subjects with active SLE who were receiving stable background therapy for SLE.

The secondary objective of the study was to assess the pharmacokinetics (PK), pharmacodynamics (PD), and immunogenicity of TAK-079 administration over a 12-week dosing period.

The exploratory objectives were to assess the effects of repeated administration of TAK-079 on SLE disease activity using clinical rating scales and biomarkers.

This phase 1b study was aimed at assessing safety, PK, PD, and immunogenicity of TAK-079. Additionally, it was designed to better understand the proof of mechanism and potential biologic activity of TAK-079 in a patient population with specific, measurable clinical manifestations of SLE. The study design allowed for subjects to continue their SLE background therapy, managed under the care of their physician, while evaluating the benefit-risk of TAK-079 as an investigational add-on therapy.

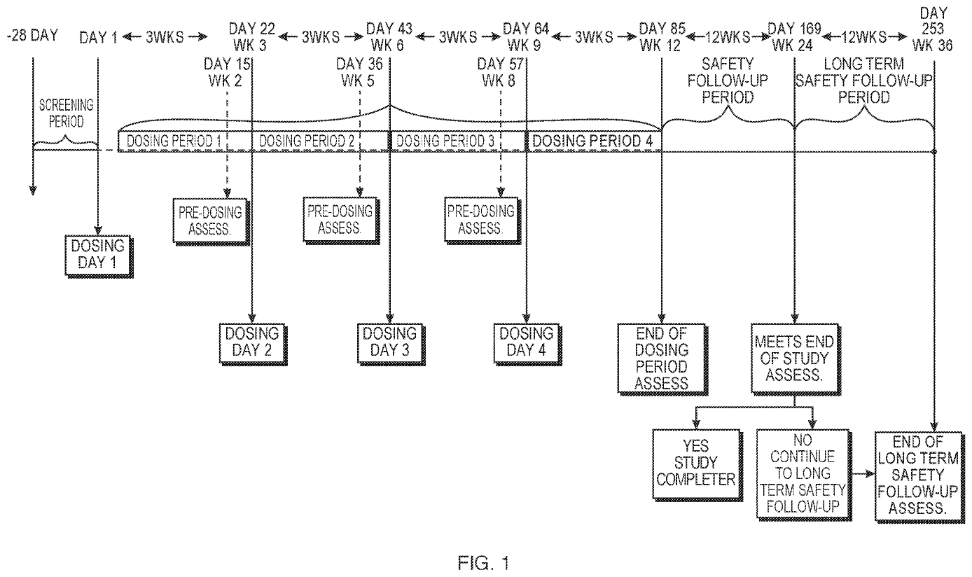

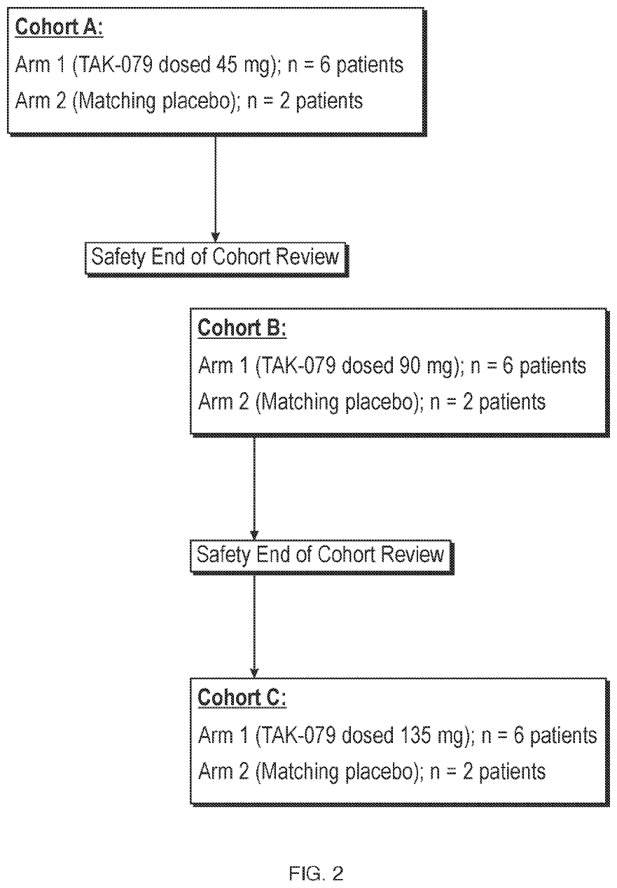

The study compared active TAK-079 with matching placebo in combination with a background SLE standard treatment across 3 sequentially enrolling cohorts in a double-blinded study design (FIG. 1 and FIG. 2). A 3:1 randomization within each cohort assigned each subject to: (1) TAK-079 administered as a single subcutaneous (SC) injection every 3 weeks for 12 weeks (total of 4 doses); or (2) matching placebo given in same route and schedule. Each cohort aimed to enroll 8 subjects; 6 subjects were randomized to TAK-079 dosing and 2 subjects to TAK-079 matching placebo. After each dosing day, subjects returned for postdose assessments as outlined in Table 4 and Table 5.

Following completion of the 12-week dosing period, subjects were assessed for an additional 12-week safety follow-up period, completing safety visits every 4 weeks and ending this postdosing study period with a safety visit on Week 24. Based on clinical assessments at this visit, subjects may have completed the study or may have advanced to the long-term safety follow-up period, for an additional on-study 12-week safety monitoring period (based on end-of-study criteria outlined in Table 13), during which time the end-of-study parameters not met and deemed by the principal investigator as study-related continued to be assessed and followed as outlined in Section of “End-of-Study Assessments”.

Sequentially enrolling the double-blind cohorts allowed for the evaluation of TAK-079 dosing in a step-wise, double-blinded design. Additionally, the double-blinded design provided support for unbiased safety and tolerability assessment as well as an intracohort and cross-cohort analysis of active TAK-079 and matching placebo for PK, PD, immunogenicity, and efficacy endpoints. (See, FIG. 2.)

### Selection of Study Population

The study population was limited to subjects with SLE who exhibited moderate to severe disease with persistent disease activity and who had not responded adequately to standard SLE background therapy treatment but had not recently had an acute flare that was moderate to severe in nature. All subjects must have been positive for anti-dsDNA antibodies and/or anti-extractable nuclear antigens (ENA) antibodies.

Based on findings from the first-in-human (FIH) study (TAK-079-101), most subjects will have detectable expression of CD38. The level of CD38 expression (quantified in molecules of equivalent soluble fluorochrome [MESF]) on PB cells was evaluated from samples obtained at screening in the TAK-079-101 study in healthy subjects. A total of 129 evaluable subjects were analyzed for expression of CD38, of whom 100% had detectable levels of CD38. The level of CD38 expression in these healthy subjects ranged approximately 5-fold from 693,474 MESF units to 3,531,222 MESF units, with a mean of 1,747,840 MESF units. The lowest CD38 expression was approximately 7-fold higher than background, indicating that healthy subjects express high levels of CD38 on the surface of peripheral blood PBs.

### Inclusion Criteria

Subjects eligible for enrollment in the study must have met the following criteria: (a) the subject understood and agreed to study participation by providing a signed and dated written ICF and any required privacy authorization before the initiation of any study procedures (as applicable, the subject's legally acceptable representative could provide written ICF in accordance to local and regional regulatory requirements) and, in the opinion of the investigator, was capable of complying with protocol requirements; (b) the subject was aged 18 to 75 years, at the time of signing the study ICF; (c) the subject was diagnosed with SLE as defined by either the 2012 Systemic Lupus International Collaborating Clinics or the American College of Rheumatology diagnostic criteria; (d) the subject had a Systemic Lupus Erythematosus Disease Activity Index 2000 (SLEDAI-2K) score ≥6; (e) the subject was positive for anti-dsDNA antibodies and/or anti-ENA antibodies; (f) the subject was receiving current concomitant medication that was consistent with medication restrictions and limitations outlined in Table 7 and Table 8, in the best medical judgment of the principal investigator, the subject was considered likely to be able to maintain dosages consistent with protocol limitations throughout the study dosing period, unless precluded by toxicity or the need for protocol-defined rescue therapy; and (g) the subject met and/or agreed to the following birth control requirements.

Birth control requirements for men: (i) sterile, or (ii) if nonsterile, agreed to (1) remain abstinent or (2) use an appropriate method of contraception, including a condom with spermicide, from study drug administration on the first day of the first dose until 90 days or 5 half-lives of study drug, whichever was longer, after the last dose of study drug administration. No restrictions were required for a vasectomized male subject provided the subject was at least 1-year postbilateral vasectomy procedure before study drug administration on first day of the first dose. A male subject whose vasectomy procedure was performed less than 4 months before study drug administration on the first day of the first dose must follow the same restrictions as a nonvasectomized man. Appropriate documentation of surgical procedure was to be provided. Agreed to not donate sperm from study drug administration on the first day of the first dose, until 90 days or 5 half-lives of study drug, whichever is longer, after the last dose of study drug administration.

Birth control requirements for women: (i) women of child-bearing potential: (1) female subjects who were of child-bearing potential must have agreed to remain abstinent or use double barrier contraception, consisting of protocol-defined, medically acceptable methods (e.g., implants, injectables, oral contraceptives, intrauterine devices) and contraception had to be used from the time of signing the ICF through 90 days or 5 half-lives of study drug, whichever was longer, after the last dose of study drug administration; or (ii) women with no child-bearing potential were defined as meeting at least 1 of the following criteria: (1) postmenopausal (defined as 12 months of spontaneous amenorrhea in women with serum follicle-stimulating hormone levels >40 mIU/mL) with appropriate documentation of follicle-stimulating hormone levels was required; (2) surgically sterile by hysterectomy and/or bilateral oophorectomy with appropriate documentation of surgical procedure; (3) had a tubal ligation with appropriate documentation of surgical procedure; or (4) has a congenital condition resulting in no uterus.

### Exclusion Criteria

Subjects meeting any of the following criteria were not eligible for study enrollment. (a) The subject had participated in another investigational study within 4 weeks or 5 half-lives of study drug, whichever was the longer, before the screening visit (the 4-week window was derived from the date of the last study procedure, and/or adverse event (AE) related to the study procedure in the previous study, to the screening visit of the current study). (b) The subject had a positive pregnancy test. (c) The subject was currently lactating/nursing or had plans to nurse during the study (to include during the 12-week safety follow-up period of the study). (d) The subject had a history of chronic alcohol or drug abuse ≤12 months before the screening visit. (e) The subject had a history of a malignant disease (except for successfully treated basal cell carcinoma, squamous cell carcinoma, or cervical carcinoma in situ)≤5 years before the screening visit. (f) Subjects had chronic obstructive pulmonary disease (COPD) or asthma with a forced expiratory volume in 1 second (FEV1)<50% of predicted normal. Note: FEV1testing was required for subjects suspected of having COPD or asthma. (g) The subject had a major surgery and/or donated or lost >1 unit of blood (approximately 500 mL) in ≤4 weeks before the screening visit. (h) The subject had an opportunistic infection 12 weeks before initial study dosing or was currently undergoing treatment for a chronic opportunistic infection, such as tuberculosis (TB), pneumocystis pneumonia, cytomegalovirus, herpes simplex virus, herpes zoster, or atypical mycobacteria. (i) The subject currently had, or recently had, an acute or chronic infection requiring 1 or more of the following interventions: (1) hospitalization ≤30 days before the screening visit or (2) administered parenteral (IV or intramuscular) antibacterial, antiviral, antifungal, or antiparasitic agents ≤30 days before the screening visit. (j) The subject had a positive T cell interferon-γ release assay (TIGRA) (resulted through QuantiFERON TB Gold test or T-Spot/Elispot) at the screening visit (to be analyzed by local laboratory), noting the following: (1) a purified protein derivative skin test could be used as a replacement, if TIGRA testing was not available, or (2) subjects with an indeterminate TIGRA result must have met the following criteria: (i) negative purified protein derivative skin test (defined as <5 mm induration), or (ii) low risk of acquiring TB (e.g., avoided close contact with TB positive individual[s]) and/or chest x-ray ≤6 months before the screening visit that was consistent with no evidence of latent or active TB. (k) The subject had drug-induced SLE or any other rheumatologic or autoimmune disease (excluding secondary Sjögren syndrome or mixed connective tissue disease). (1) The subject required therapeutic intervention 60 days before initial study dosing for active neuropsychiatric SLE, as demonstrated by, but not limited to, the following: (1) new or worsening impaired level of consciousness, (2) psychosis, (3) delirium or confusional state, (4) grand mal seizure (including status epilepticus), (5) aseptic meningitis, (6) ascending or transverse myelitis, or (7) chorea, cerebellar ataxia, or demyelinating syndromes. (m) The subject had an active glomerulonephritis (i.e., a concurrent acute renal flare or documented acute renal flare in the previous 3 months that required lupus nephritis induction therapy) that met at least 1 of the following criteria: (1) proteinuria (protein >3000 mg/24 hours), (2) urine protein-to-creatinine ratio of >300 mg/mmol, or (3) an estimated glomerular filtration rate (GFR)<30 mL/min/1.73 m² (as calculated by the Modification of Diet in Renal Disease [MDRD] formula; see Section of “Laboratory Evaluations”). (n) The subject had at least 1 of the following laboratory test values: (1) alanine aminotransferase or aspartate aminotransferase >3 times the upper limit of normal, (2) total bilirubin >1.5 times upper limit of normal (Note: Subjects with a confirmed diagnosis of Gilbert syndrome that was documented in the subject's medical record were not excluded based on this criterion), (3) platelets <75,000/mm³, (4) neutrophils <1500/mm³, (5) hemoglobin <8 g/dL, or (6) IgG less than lower limit of normal. (o) The subject had a positive test result for hepatitis B surface antigen or hepatitis C antibody, or HIV antibody/antigen, at screening. (p) The subject had a concurrent medical condition that, in the opinion of the investigator, could confound interpretation of results or affect the subject's ability to fully participate in the study. (q) The subject had a history of severe allergic or anaphylactic reactions to recombinant proteins or excipients used in the TAK-079 formulation.

Removal of Subjects from Therapy or Assessment

Laboratory findings in which TAK-079 dosing was to be temporarily held, or permanently discontinued, are outlined in Table 11. Subjects exhibiting these or other clinical findings for which the principal investigator considered that continued TAK-079 dosing may place the subject at undue risk were immediately held from further TAK-079 dosing and continued to be followed for safety. In such instances, TAK-079 could be resumed under consultation with the study medical monitor (MM) and in accordance with protocol-defined dosing criteria.

Treatment with study drug could be discontinued permanently for any of the following reasons: (a) AE and/or serious adverse event (SAE); (b) withdrawal by subject; (c) protocol violation; (d) study terminated by sponsor; (e) lost to follow-up; or (f) other.

Once study drug had been discontinued, all study procedures outlined for follow-up were completed as specified in the schedule of events (SOE) (Table 4 and Table 5). Subjects withdrawing from study treatment were asked if they would permit follow-up assessment or not. If the subject completely withdrew consent, no further follow-up assessments were completed. The primary reason for study drug discontinuation was recorded on the electronic case report form (eCRF).

TABLE 4
Schedule of Study Events: Dosing Period

| Study Procedures | | | | | | | | |
|---|---|---|---|---|---|---|---|---|
| | Dosing | | | | Pre- | Dosing | | |
| Screening | V1 | HV1 | HV2 | HV3 | dose V1 | V2 | HV4 | HV5 |
| Study day (window [days])^{b} | | | | | | | | |
| | | D 2 | D 5 | D 8 | D 15 | | D 23 | D 26 |
| −28-1 | D 1 | (+1) | (+1) | (+1) | (+1) | D 22 | (+1) | (+1) |

Laboratory Assessments (NOTE: See Table 10 for additional laboratory draws)

| | | | | |
|---|---|---|---|---|
| Informed consent | X | | | |
| Inclusion and | X | | | |
| exclusion criteria | | | | |
| Medical history | X | | | |
| including SLE | | | | |
| SLE assessment^{c} | X | X^{d} | | X^{d} |
| Physical | X | X^{d} | | X^{d} |
| examination^{e} | | | | |
| Vital signs^{f} | X | X | | X |
| Dosing criteria | | X^{d} | X | X^{d} |
| assessment | | | | |

| | |
|---|---|
| Concomitant | Recorded ≤28 days before the first dose of TAK-079, through completion of final study safety visit |
| medications | (i.e., final safety follow-up visit, or when applicable, the final long-term safety follow-up visit). |
| AE assessments^{g} | (Serious) AEs (to include pretreatment, and related and unrelated treatment-emergent events) are to |
| | be recorded from the time of signing the ICF through to completion of the final study safety visit |
| | (ie, final safety follow-up visit, or when applicable, the final long-term safety follow-up visit). |

Dosing

| | | | | | | |
|---|---|---|---|---|---|---|
| Premedication | | X^{d} | | | | X^{d} |
| dosing^{h} | | | | | | |
| TAK-079 dosingⁱ | | X | | | | X |
| Pregnancy test^{j} | X | X^{d} | | | | X^{d} |
| Hematology | X | | | | X | |
| Chemistries | X | | | | X | |
| Urinalysis^{k} | X | X^{d} | | | X | X^{d} |
| Immune profiling | X | | | | X | |
| Circulating | | X^{d} | X | X | | X^{d} |
| biomarkers^{l} | | | | | | |
| Cytokine markers^{m} | | X^{d} | | | | X^{d} |
| SLE Abⁿ | X | X^{d} | | | | X^{d} |
| Quantitative | X | | | | X | |
| IgA/IgM/IgG | | | | | | |
| Immunogenicity | | X^{d} | | | | X^{d} |
| assessments (to | | | | | | |
| include ADA) | | | | | | |

| Study Procedures | | | | | | | | |
|---|---|---|---|---|---|---|---|---|
| | | | | | | | | End of |
| | Pre- | Dosing | | Pre- | Dosing | | | Dosing |
| HV6 | dose V2 | V3 | HV7 | Dose V3 | V4 | HV8 | HV9 | Visit^{a} |
| Study day (window [days])^{b} | | | | | | | | |
| D 29 | D 36 | | D 50 | D 57 | | D 71 | D 78 | D 85 |
| (+1) | (+1) | D 43 | (+1) | (+1) | D 64 | (+1) | (+1) | (+1) |

Laboratory Assessments (NOTE: See Table 10 for additional laboratory draws)

| | | | | | |
|---|---|---|---|---|---|
| Informed consent | | | | | |
| Inclusion and | | | | | |
| exclusion criteria | | | | | |
| Medical history | | | | | |
| including SLE | | | | | |
| SLE assessment^{c} | | X^{d} | | X^{d} | X |
| Physical | | X^{d} | | X^{d} | X |
| examination^{e} | | | | | |
| Vital signs^{f} | | X | | X | X |
| Dosing criteria | X | X^{d} | X | X^{d} | |
| assessment | | | | | |

| | |
|---|---|
| Concomitant | Recorded ≤28 days before the first dose of TAK-079, through completion of final study safety visit |
| medications | (i.e., final safety follow-up visit, or when applicable, the final long-term safety follow-up visit). |
| AE assessments^{g} | (Serious) AEs (to include pretreatment, and related and unrelated treatment-emergent events) are to |
| | be recorded from the time of signing the ICF through to completion of the final study safety visit |
| | (ie, final safety follow-up visit, or when applicable, the final long-term safety follow-up visit). |

Dosing

| | | | | | |
|---|---|---|---|---|---|
| Premedication | | X^{d} | | X^{d} | |
| dosing^{h} | | | | | |
| TAK-079 dosingⁱ | | X | | X | |
| Pregnancy test^{j} | | X^{d} | | X^{d} | X |
| Hematology | X | | X | | X |
| Chemistries | X | | X | | X |
| Urinalysis^{k} | X | X^{d} | X | X^{d} | X |
| Immune profiling | X | | X | | X |
| Circulating | | X^{d} | | X^{d} | X |
| biomarkers^{l} | | | | | |
| Cytokine markers^{m} | | X^{d} | | X^{d} | X |
| SLE Abⁿ | | X^{d} | | X^{d} | X |
| Quantitative | X | | X | | X |
| IgA/IgM/IgG | | | | | |
| Immunogenicity | | X^{d} | | X^{d} | X |
| assessments (to | | | | | |
| include ADA) | | | | | |

Ab: antibody; ADA: antidrug antibodies; AE: adverse event; CLASI: Cutaneous Lupus Erythematosus Disease Area and Severity Index; CRS: cytokine release syndrome; D: study day; dsDNA: double-stranded DNA; ENA: extractable nuclear antigen; HV: home visit; ICF: informed consent form; IgA: immunoglobulin A; IgG: immunoglobulin G; IgM: immunoglobulin M; IRB: institutional review board; SLE: systemic lupus erythematosus; VAS: visual analog scale.
^{a}After completing Week 12 of study dosing, subjects will continue in the study for a 12-week safety follow-up period as outlined in Table 5.
^{b}Additional time is allowed for extenuating circumstances, as granted on approval of medical monitor or designee.
^{c}SLE assessments are to include the following evaluations: CLASI, Systemic Lupus Erythematosus Disease Activity Index 2000, 44-Joint Assessment, and the Physician’s Global Assessment of Disease (VAS); assessments are to be completed at screening and each dosing day, before each dose.
^{d}To be performed before dose administration.
^{e}Physical examinations are to be symptom-and SLE disease directed with significant clinical findings noted as AEs.
^{f}Vital signs, including temperature, pulse, respiratory rate, and blood pressure, are to be assessed before each study dose and 4 hours after dosing, as part of postinjection assessments; vital signs should also be assessed at any time it is clinically warranted, either during clinic or home study visits (i.e., in instances where the subject exhibits signs or symptoms of injection reaction, CRS, or hypersensitivity reactions). See Section of “Vital Signs” for further details.
^{g}Assessments for AEs arc to include a symptomatic examination of the subject’s current discase state, conditions, and treatments (scc Section of “AEs”).
^{h}Premedication dosing and dosages are outlined in Section of “Premedication”.
ⁱThe time and the anatomical site of injection are to be recorded for each study dose; subjects should be closely monitored in the clinic for at least 4 hours after each dose; before discharge all subjects must receive information on signs and symptoms of anaphylactic reactions and CRS.
^{j}Pregnancy testing is done. Additional testing must be assessed if the subject’s menstrual period is delayed, or if the IRB requests.
^{k}Urinalysis, including protein-to-creatinine ratio and nitrates, will be performed by central laboratory 1 week before dosing; additional protein and nitrate assessments are to be performed by local laboratory (i.e., dipstick) on study dosing days, before dosing; abnormal findings by local laboratory assessment must be confirmed by central laboratory evaluation. Further detail is provided in Section of “Laboratory Evaluations”.
^{l}Circulating biomarkers are to include assessment of complement C3 and C4 levels.
^{m}Additional samples of cytokine markers are to be drawn if CRS is suspected; additional PK and biomarker sampling may be requested.
ⁿSLE antibodies include, but are not limited to, antiphospholipid, anti-dsDNA, and anti-ENA antibodies.

TABLE 5
Schedule of Study Events: Safety Follow-up and Long-term Safety Follow-up Periods

| Safety Follow-up Period | | | Long-Term Safety Follow-up Period^{a} | | |
|---|---|---|---|---|---|
| | | Safety/Early End- | Long-term | Long-term | Long-term End- |
| Safety | Safety | of-Study Visit^{a} | Safety | Safety | of-Study Visit |
| V1 | V2 | V3 | V1 | V2 | V3 |
| Study Week | | | | | |
| Week 16 | Week 20 | Week 24 | Week 28 | Week 32 | Week 36 |

| | | | |
|---|---|---|---|
| SLE assessment | X | X | X |
| tools^{b} | | | |

| | |
|---|---|
| Concomitant | Recorded <28 days before the first dose of TAK-079 through completion |
| medications | of final study safety visit (i.e., final safety follow-up visit, or if |
| | applicable, at the final long-term safety follow-up visit) |
| AE assessmentc | (Serious) AEs (includes pretreatment, and related and unrelated treatment- |
| | emergent events) are to be recorded from the signing of the ICF through |
| | completion of final study safety visit (i.e., final safety follow-up visit, |
| | or if applicable, the final long-term safety follow-up visit) |

Laboratory Assessments

| | | | |
|---|---|---|---|
| Pregnancy test^{d} | | | X |
| Hematology | X | X | X |
| Chemistries | X | X | X |
| Urinalysis^{e} | X | X | X |

AE: adverse event; CLASI: Cutaneous Lupus Erythematosus Disease Area and Severity Index; HV: home visit; ICF: informed consent form; IRB: institutional review board; SLE: systemic lupus erythematosus; V: visit; VAS: visual analog scale.
^{a}Clinical parameters that do not meet the end-of-study criteria, as outlined in Table 10, and are deemed by the principal investigator as study related, will continue to be assessed no less than every 4 weeks until resolution, as outlined in Section of “End-of-Study Assessments”.
^{b}SLE assessment tools include: CLASI, Systemic Lupus Erythematosus Disease Activity Index 2000, 44-Joint Assessment, and the Physician’s Global Assessment of Disease (VAS).
^{c}AE assessments are to include a symptomatic examination of the subject’s current disease state, conditions, and treatments.
^{d}Pregnancy testing is done. Additional testing must be assessed if menstrual period is delayed, or at IRB request.
^{e}Urinalysis, including protein-to-creatinine ratio and nitrates, will be performed by central laboratory; additional protein and nitrate assessments are to be performed on each clinic visit; abnormal findings by local laboratory assessment must be confirmed by central laboratory evaluation. Further detail is provided in Section of “Laboratory Evaluations”.

### Collection of Demographic and Medical History Data

A complete medical history was compiled for each subject during the screening period (i.e., ≤28 days before study Day 1) and included assessment and documentation of prior medical history, comorbidities, and concomitant treatments. This included assessments of current SLE signs, symptoms, morbidities, as evaluated and scored by disease activity tools and previous and current SLE therapies (see Section of “Efficacy Measurements”). Demographics included the date of birth, race, ethnicity, and sex of the subject during screening.

### Treatments

### Treatments Administered

TAK-079 or matching placebo was administered as a single SC injection every 21 days (i.e., 3 weeks) over the course of 12 weeks for 4 total doses as part of each study cohort. Subjects in cohorts A, B, and C received 45, 90, and 135 mg TAK-079, respectively, or matching placebo.

The selection of doses and frequency of administration of TAK-079 were based on a comprehensive review and analysis of data derived from the following (1) dosing of healthy subjects with TAK-079 (Study TAK-079-101); (2) nonclinical repeat TAK-079 dosing studies (TAK-079-10015, TAK-079-1018, and TAK-079-10019) in cynomolgus monkeys; and (3) data derived from repeat dosing of daratumumab (Daralex®), a related anti-CD38 cytolytic antibody that is licensed for treatment in multiple myeloma.

The starting dose of 45 mg for the initial dosing cohort (i.e., Cohort A) was selected based on the favorable safety profile and PD target effect (i.e., a sustained reduction of PBs) observed after a 0.6 mg/kg dose was administered to healthy subjects (Study TAK-079-101). A single SC dose of 0.6 mg/kg dose reduced the level of PBs in peripheral blood >90% and NK cells >80% without comparable reductions in monocytes and B and T cells. Levels of PBs and NK cells recovered to 50% of baseline levels 21 days after administration, on average. At this dose, there were no SAEs, on-study deaths, or AEs that led to study discontinuation. No remarkable findings for laboratory tests, electrocardiogram (ECGs), vital signs, or physical examinations were reported that were related to TAK-079 administration. Two subsequent dosing cohorts were planned at doses of 90 mg (2-fold increase from the first dose cohort) and 135 mg (a 50% increase from the previous cohort). Because patients with lupus generally exhibit higher levels of PBs expressing CD38 than those seen in healthy study subjects, higher doses may be needed to achieve comparable PD effects; subjects were carefully monitored to maintain a balance of benefit-risk.

### Identity of Study Drugs

Study drug was supplied as 100 mg TAK-079 in 1 mL (100 mg/mL) of aqueous solution of histidine, histidine hydrochloride monohydrate, sucrose, polysorbate 20, and pH 5.9 buffer, to be administered as a single SC injection (Table 6).

TABLE 6
Identity of Study Drug

| Study | Product Dose | Study | Mode of |
|---|---|---|---|
| Drugs | Strength and Form | Dosage | Administration |
| TAK-079 | 100 mg/mL solution | 45 mg/90 mg/ | SC |
| | | 135 mg | |
| Placebo | NA | NA | SC |

NA: not applicable; SC: subcutaneous.

### Packaging, Labeling, and Storage

TAK-079 drug product and matching placebo were supplied in aseptically filled, single-use, clear, type I, borosilicate glass vials with fluoropolymer-coated butyl rubber stoppers and aluminum crimp seals with flip-off caps.

Supplies of TAK-079 and matching placebo were labeled according to the current ICH guidelines on GCP and Good Manufacturing Practices and included any locally required statements.

TAK-079 and matching placebo were stored according to the manufacturer's stipulation, as specified on the label, and remained in the original container until dispensed. A daily temperature log of the drug storage area was maintained every day.

### Study Reference Product Composition

The matching placebo product consisted of histidine, histidine hydrochloride monohydrate, sucrose, polysorbate 20, and pH 5.9 buffer administered via SC injection following the same procedures as for the TAK-079 drug product. The Process B mezagitamab drug product is made in 2 strengths, 5 mg/mL or 100 mg/mL. Each strength is a clear-to-opalescent, colorless-to-brownish-yellow solution containing mezagitamab in an aqueous solution of histidine, histidine hydrochloride monohydrate, sucrose, polysorbate 20, and water for injection at approximately pH 5.9. The Process B placebo is a clear, colorless solution containing an aqueous solution of histidine, histidine hydrochloride monohydrate, sucrose, polysorbate 20, and water for injection at approximately pH 5.9. The Process B mezagitamab drug product and placebo are supplied in aseptically filled, single use, clear, type I, borosilicate glass vials with fluoropolymer coated butyl rubber stoppers and aluminum crimp seals with flip-off caps.

### Method of Assigning Subjects to Treatment

The assignment of study subjects to 1 of 2 study arms within each sequentially-enrolled cohort was maintained through a blinded randomization schedule, generated and maintained by an interactive voice/web response system (IXRS).

### Selection and Timing of Dose for Each Subject

Since infusion reactions and other antibody-mediated hypersensitivity reactions had been reported with other biologic agents, similar AEs could have occurred following treatment with TAK-079. To prevent potential infusion-related reactions (IRRs), premedication was mandatory for all patients before each study dose. Postdose medication was to be administered at the investigator's discretion to further minimize delayed IRRs as clinically indicated.

### Premedication

On each dosing day, 1 to 3 hours before TAK-079 administration, subjects were premedicated with a regimen consistent with, but not limited to, the following: (1) antipyretic: oral acetaminophen (650 to 1000 mg) and (2) antihistamine: oral or IV diphenhydramine (25 to 50 mg, or equivalent). The clinical site was responsible for sourcing premedications outlined in the protocol.

### Study Drug Dosing

After subjects received premedication treatment, the TAK-079 dose or matching placebo was administered with a syringe as SC injections up to a maximum volume of 2 mL, such that the full schedule dose was administered. The time and anatomical site of SC injection was recorded for each dose, with the site of injection rotated for each dose (injection sites of the abdomen, thighs, arms, and upper buttock area are acceptable).

### Postdose Medications

Subjects could receive low-dose methylprednisolone (<20 mg) for the prevention of delayed injection-related reaction, as clinically indicated, and under the discretion of the principal investigator.

Subjects with a higher risk of respiratory complications (e.g., subjects with a history of COPD and subjects with asthma) could be administered the following, after each study dose (at the investigator's discretion) to further prevent IRRs: (a) an antihistamine (diphenhydramine or equivalent) on the first and second days after study dosing; (b) a short-acting P2-adrenergic receptor agonist, such as salbutamol (albuterol) aerosol, or (c) control medications for lung disease, such as the following: (i) inhaled corticosteroids with or without long-acting p2-adrenergic receptor agonists for subjects with asthma, or (ii) long-acting bronchodilators, such as tiotropium or salmeterol with or without inhaled corticosteroids, for subjects with COPD.

Based on emerging data, the Takeda physician/designee could enhance treatments administered pre- or post-TAK-079 injection to ensure subject safety.

### Blinding

The randomization and medication schedules were generated and maintained by an IXRS. All randomization information was stored in a secured area, accessible only by authorized personnel.

To maintain the integrity of the study, all study personnel, including the investigators, site personnel, the contract research organization MM, study clinicians, and the sponsor, were blinded to the treatment assignments during the treatment period. Treatment assignments were obtained through the IXRS according to the procedures outlined in the study manual. Information regarding the treatment assignments was kept securely at Takeda or designee, per its standard operating procedures.

Records of the subject number, the date the study drug was dispensed, and the treatment assignment were maintained by the study site.

Emergency unblinding, if necessary, was conducted via the IXRS. There were no incidences of emergency unblinding during this study.

Cohort A and B treatment assignments were unblinded after all subjects in Cohorts A and B had completed the study and all data had been source data verified. This unblinding was deemed appropriate as, at the time of unblinding, (1) Cohort A and B subjects had all finished participation in the study (i.e., completed the study or discontinued the study early); (2) the ongoing Cohort C followed a separate randomization schedule and therefore was not impacted; (3) information collected in this interim analysis was unlikely to introduce bias to the ongoing Cohort C for this small and early phase study; and (4) the initial statistical analysis plan (SAP) had been finalized and approved.

### Prior and Concomitant Therapy

### Background SLE Therapy

Eligible subjects received SLE background therapy for ≥12 weeks (with stable dosing ≥8 weeks) before screening. Once enrolled into the study, subjects remained on background therapy, as managed by their principal investigator, in accordance with local institutional practices, and in alignment with the study protocol, throughout study participation. Background SLE therapy ongoing at the time of screening was recorded in the eCRF, as were any changes to this therapy.

### Rescue Therapy

Rescue therapy was defined as additional dosing of concomitant medications in accordance with institutional practices or the physician's best medical judgment to control and manage underlying SLE conditions.

Subjects remained on their stable dose of immunosuppressive and corticosteroid therapies throughout the study. Decreases in the dose of immunosuppressants, due to toxicity, were allowed at the discretion of the investigator. Increasing, adding, or changing background immunosuppressive therapy, or adding a medication not otherwise within protocol limits, as deemed necessary by the principal investigator to treat manifestations of SLE, resulted in discontinuation of the subject from study dosing and advancement to safety follow-up; these subjects will be categorized as study nonresponders.

NOTE: Subjects who required an increase in corticosteroid dose above 0.5 mg/kg/day (or 40 mg/day of prednisone) or equivalent, for control of SLE activity, or management of a new SLE flare during the study, were discontinued from study drug. The prednisone dosage or equivalent could be increased to a dose that was less than or equal to 0.5 mg/kg/day or 40 mg/day, whichever is lower. However, dosing should have been tapered to the preflare level or 20 mg/day, whichever was greater, within 4 weeks or less. If tapering was not possible AND a higher dose was needed to treat SLE manifestations, the subject was discontinued from study drug, advanced to safety follow-up, and was designated as a study nonresponder.

### Excluded and Dose-Limited Medications

Excluded and dose-limited concomitant medications are provided in Table 7 and Table 8.

TABLE 7
Excluded Medications

| | Exclusion Criteria |
|---|---|
| | Exclusion Criteria Were Upheld Throughout Study Participation, |
| Category or Agent | in Addition to the Time Points Indicated Below)^{a} |
| Belimumab, abatacept, | <3 months before the screening visit |
| or tocilizumab | |
| Cyclophosphamide | Restricted from <12 weeks before initiation of on-study dosing |
| Immunosuppressant | To include calcineurin-inhibitors (e.g., systemic cyclosporine and |
| regimen | tacrolimus), leflunomide, to be excluded throughout study |
| | participation. Must be discontinued at least 3 months before |
| | screening visit. |
| Live vaccines | All vaccines: Maintain restrictions as follows and throughout study |
| | participation, until 12 weeks following the last dose of study dosing |
| | Live vaccines: 90 days before initial dosing on study |
| | Day 1 Inactivated vaccines: 30 days before initial dosing on study |
| | Day 1 |
| Rituximab or other cell | <6 months before the screening visit |
| depleting biological | |
| agents | |
| Alemtuzumab | No previous or concurrent dosing while on study |

AE: adverse event.
^{a}Exceptions to excluded medications could be allowed for treatment of AEs after discussion and agreement between the sponsor and principal investigator.

TABLE 8
Dose-Limited Concomitant Medications

| | Dosing Criteria |
|---|---|
| | (Criteria Were Maintained from Screening to Completion of |
| Medications | Study Dosing Phase) |
| Immunosuppressants^{a} | Generally, agents must be dosed for at least 12 weeks before |
| | screening, with stable dosing for at least 8 weeks before the first |
| | dose of TAK-079 or matching placebo. |
| | Regimens consisting of <2 agents while on study (not applicable |
| | to topical (Table 7). |
| Azathioprine | 1 to 2 mg/kg/day |
| Mycophenolate mofetial | 1 to 2 g/day |
| Methotrexate | 7.5 to 25 mg/week |
| Corticosteriod^{a} | Stable dosing equivalent to <20 mg/day of oral prednisone |
| | maintained for >28 days before the first dose of TAK-079 or |
| | matching placebo. |
| Alemtuzumab | Stable dosing maintained for >28 days and is likely to maintain |
| | the current dose throughout the dosing period, as determined by |
| | the principal investigator. |

^{a}Dose limitations not applicable to topical agents.

### Documentation of Concomitant Medication

Concomitant medications, blood products, and procedures were recorded from the first dose of TAK-079 or matching placebo, through the end of the safety follow-up period (i.e., end of safety follow-up visit in Week 24 or, when applicable, end of long-term safety follow-up visit in Week 36). Trade name and international nonproprietary name (if available), indication, and start and end dates of the administered medication were recorded.

### Treatment Compliance

Study drug is administered in clinic. Treatment compliance will be calculated as outlined in Section of “Extent of Exposure and Compliance”

### Efficacy, Pk, Pd, Biomarker and Safety Variables

### Measurements Assessed and Flow Chart

Schedules of study procedures are presented for the dosing period in Table 4 and for the safety/long-term safety follow-up periods in Table 5. Additional information on PK; CD38; T lymphocyte, B lymphocyte, and NK cells samples; and ECG times is provided in Table 9.

TABLE 9
PK, CD38, TBNK Samples, and ECG Times by Visit

| | Study | Specialty Laboratory Sample Times | | | | |
|---|---|---|---|---|---|---|
| Visit | Day | TBNK | CD38 | RNA | PK^{c} | ECG Assessments^{a} |
| Screening | NA | | | | | X |
| Dosing | DI | X^{b} | X^{b} | X^{b} | All 3 visit time points as follows: | X^{b} |
| Visit 1 | | | | | Any time point before dosing | Pre-dose |
| | | | | | 2 hr (±1 hr) after dosing | 5 hr after dosing |
| | | | | | 5 hr (±1 hr) after dosing | (before PK draw) |
| HVI | D 2 | X | X | X | 33 hr (±9 hr) after dosing | X |
| HV2 | DS | X | X | X | 108 hr (±12 hr) after dosing | X |
| HV3 | D 8 | | | | 168 hr (±24 hr) after dosing | |
| Predose Visit 1 | D 15 | X | X | X | Any time during visit | |
| Dosing | D 22 | X^{b} | X^{b} | X^{b} | All 3 visit time points as follows: | X^{b} |
| Visit 2 | | | | | Any time point before dosing | |
| | | | | | 2 hr (±1 hr) after dosing | |
| | | | | | 5 hr (±1 hr) after dosing | |
| HV4 | D 23 | X | X | X | 33 br (±9 hr) after dosing | X |
| HV5 | D 26 | X | X | X | 108 hr (±12 hr) after dosing | |
| HV6 | D 29 | | | | Any time during visit | |
| Predose Visit 2 | D 36 | X | X | X | Any time during visit | |
| Dosing | D 43 | X^{b} | X^{b} | X^{b} | Both visit time points as follows: | X^{b} |
| Visit 3 | | | | | Any time point before dosing | |
| | | | | | 5 hr (±1 hr) after dosing | |
| HV7 | D 50 | X | X | X | Any time during visit | X |
| Predose Visit 3 | D 57 | X | X | X | Any time during visit | |
| Dosing | D 64 | X^{b} | X^{b} | X^{b} | Both visit time points as follows: | X^{b} |
| Visit 4 | | | | | Any time point before dosing | |
| | | | | | 5 hr (±1 hr) after dosing | |
| HV8 | D 71 | X | X | X | Any time during visit | X |
| HV9 | D 78 | | | | Any time during visit | |
| End-of-dosing | D 85 | X | X | X | Any time | X |
| visit | | | | | during visit | |
| End-of-safety | Week | | | | | X |
| follow-up visit | 24 | | | | | |

D: day; ECG: electrocardiogram; hr; hour; HV; home visit; MM: medical monitor NA: not applicable; PK: pharmacokinetic; TBNK: T lymphocyte, B lymphocyte, and natural killer cells.
^{a}ECGs must be obtained just before PK sampling when scheduled on the same study days; as part of study screening and at the end of safety follow-up visit, a single ECG must be obtained and assessed (read locally); during study-treatment, triplicate 12-lead ECGs should be obtained, (read locally), with a copy submitted centrally for future analysis. (see Section of ECGs”).
^{b}To be performed before study dosing.
^{c}Additional PK draws may be requested by the MM.

### Efficacy Measurements

The study principal investigator or appropriately trained delegated study site staff assessed each subject for disease activity based on SLE disease activity scales in accordance with the SOE study activity tables (Table 4 and Table 5).

### SLEDAI-2K

The SLEDAI-2K disease assessment tool uses 24 items, of which 16 are clinical and 8 are based solely on laboratory results (urinary casts, hematuria, proteinuria, pyuria, low complement levels, increased DNA binding, thrombocytopenia, and leukopenia). A manifestation was recorded if it presented ≤10 days from the time of assessment, regardless of severity or whether it improved or worsened. Individual item scores range from 1 to 8, with a total score ranging from 0 to 105. See e.g. Gladman et al. (2002). The Journal of rheumatology, 29(2):288-291.

For purposes of this study, items for which laboratory results were not available were scored as negative or normal.

### Cutaneous Lupus Erythematosus Disease Area and Severity Index

The Cutaneous Lupus Erythematosus Disease Area and Severity Index (CLASI) consists of 2 scores: the first summarizes the activity of the disease, whereas the second is a measure of the damage done by the disease. Activity is scored based on erythema, scale/hyperkeratosis, mucous membrane involvement, acute hair loss, and nonscarring. Damage is scored in terms of dyspigmentation and scarring (to include scarring alopecia). Dyspigmentation related to SLE lesions which remains visible for more than 12 months is classified as permanent and scored double.

### 44-Joint Assessment

The 44-Joint Assessment assesses the number of tender and swollen joints, by location, as outlined in the disease assessment tool.

### Physician's Global Assessment of Disease (Visual Analog Scale)

The Physician's Global Assessment (PGA) of disease is a visual analog scale (VAS) that reflects the clinician's judgment of overall SLE disease activity. The disease activity index scored on a VAS ranges from 0 to 3, with an increase of ≥1.0 since the last visit indicating a flare.

### PK Measurements

Blood samples were collected by venipuncture or indwelling catheter at the time points detailed in the SOE (Table 4) for the measurement of serum concentrations of TAK-079.

Samples were tested at a central laboratory.

The timing of samples could be modified during the study based on emerging PK data if a change in the sampling scheme was considered necessary to better characterize the PK profile of TAK-079. Additional PK samples could be requested if deemed necessary by the MM for specific events of clinical interest or AEs.

### Immunogenicity Samples

Serum samples for the measurement of anti-TAK-079 antibody (i.e., antidrug antibody [ADA]) are collected at multiple time points as specified in the SOE (Table 4).

The samples were taken before each dosing. The immunogenicity samples were screened for potential ADA positive samples, which were then further verified for true positivity and titer by the bioanalytical laboratory. The relationships among immunogenicity response, efficacy, and safety may be explored.

### Biomarker Measurements

In this study, several biomarkers were assessed to test for correlation with safety, PK, and, if possible, with efficacy. Biomarkers of disease activity included anti-dsDNA, anti-ENA antibodies, complement C3, C4, urine protein-to-creatinine ratio, and/or markers of CD38 pathway modulation. These biomarkers are intended to be used to identify subjects who have a higher probability of response or adverse reactions to TAK-079.

Blood samples were collected by venipuncture or indwelling catheter at the time points detailed in the SOE (Table 4) for the measurement of biomarkers. Samples were tested at a central laboratory.

### PK, IgG, and Autoantibody Quantification

TAK-079 serum concentrations were quantified through a clinically validated electrochemiluminescence immunoassay. The lower limit of quantification (LLOQ) for this assay was 5 ng/mL. IgG measurements were obtained from Roche Cobas 8000 analyzers at a central laboratory. SLE associated autoantibodies were quantified via Thermo Scientific Phadia 250 Analyzers at a central laboratory using clinically validated enzyme linked immunoassay methodology.

### CyTOF Analysis

CyTOF analysis was conducted at CellCarta (Fremont CA). Samples were run in batches of approximately ten. Each sample was thawed and washed, stained with a dye indicating cell viability, then hybridized with the defined antibody panel. Following wash, samples were fixed and incubated with a DNA intercalating agent for 3-7 days at 4° C. Fixative was then removed, and samples were suspended in water for CyTOF analysis. 100,000-250,000 events were analyzed per sample. In addition to viability assessments, we performed an immuno-oncology panel comprising 39 metal ion-coupled antibodies. This panel was modified to include TSF-19 as a noncompetitive antibody against CD38. Samples were obtained at baseline (during screening, up to 28 days prior to mezagitamab initiation) and at Days 15, 36, 57 and 85. Data were collected as FCS plots and assessed as randomly selected analysis of 100,000 cells per patient sample.

Cluster identification and analysis was done using the R package CyTofWorkflow (Nowicka et al. (2017) F1000Res 6: p. 748). FlowSOM (Van Gassen et al. (2015) Cytometry A 87(7): p. 636-45), and ConsensusClusterPlus (Wilkerson, M. D. and D. N. Hayes (2010) Bioinformatics 26(12): p. 1572-3.) were utilized to identify distinct cell populations based on their expression profiles, and results were visualized using TSNE plots (Amir el et al. (2013) Nat. Biotechnol, 31(6): p. 545-52). FlowSOM employed a self-organizing map algorithm to cluster cells into metaclusters, and ConsensusClusterPlus employed a consensus clustering approach to identify stable clusters. Additional analysis was conducted in-house. Data were expressed as median CD38 expression and % of parent population (+/− standard error of mean, when applicable). Statistical analyses and figure generation were conducted in Excel, GraphPad Prism 9, and R code. Flow cytometry panels were generated using FlowJo 10.

### Safety Measurements

Safety measurements were performed at the times specified in the SOE (Table 4 and Table 5).

Takeda clinicians conducted reviews of AEs, SAEs, and related clinical parameters to ensure consistency with an acceptable benefit-risk ratio throughout the study. At the subject level, safety and tolerability were closely monitored for each subject at the completion of each dose and before the next subsequent dose to ensure clinical dosing criteria were met before each subsequent administration of TAK-079 or matching placebo. Furthermore, a comprehensive safety assessment was conducted at the end of treatment in each cohort, which allowed for decisions regarding enrollment in subsequent cohorts. A 12-week postdose safety follow-up period allowed for continued observation and assessment of safety and sustainability of biologic activity. Subjects whose safety parameters did not return to adequate protocol-defined rebound levels by the end of the safety follow-up period progressed to a long-term follow-up period for continued monitoring (Table 5, Table 11). These continued safety observation periods provided optimal oversight and assessment of any ongoing study-related sequela.

### Physical Examination

A symptom-directed physical examination with assessments for SLE signs and symptoms was completed in accordance with standard of care.

### Vital Signs

Vital signs included body temperature, pulse, respiratory rate (RR), and blood pressure (BP). Vital signs were assessed before each study dose and 4 hours after dosing, as part of postdose assessments. Pulse and BP were assessed in similar positions at each study assessment.

In addition to the assessments scheduled in the SOE, vital signs were also assessed at any time it was clinically warranted, either in the clinic or home visit setting (i.e., subject exhibited signs or symptoms of injection reaction, CRS, or hypersensitivity reactions).

Clinically significant values, as determined by the principal investigator, were documented as an AE and closely monitored for follow-up.

NOTE: Additional vital signs to include BP measures were assessed any time a subject reported symptoms consistent with an infusion reaction. If the subject experienced hypotension (with or without symptoms), intensive BP monitoring according to local practice was instituted. The subject was not released from the study site until BP values had returned to Grade 1 or baseline for at least 1 hour.

### Laboratory Evaluations

Unless otherwise stated in the protocol, all laboratory samples, with exception to Coombs tests, were sent to the study central laboratory for analysis. Coombs test was performed locally.

Clinical chemistry and hematologic assays are outlined Table 10 and urinalysis assays are outlined in Table 12. The timings of these assessments are outlined in the SOE (Table 4 and Table 5).

TABLE 10
Clinical Chemistry and Hematology Assessments

| Hematology | | Serum Chemistry |
|---|---|---|
| ANC | Albumin | CRP |
| Hematocrit | ALP | Glucose (nonfasting) |
| Hemoglobin | ALT | LDH |
| Platelet count | AST | Potassium |
| WBC count | Bilirubin (total) | Sodium |
| WBC with differential | BUN | Total protein |
| Coagulation panel | Calcium | Urate (uric acid) |
| Coombs test (both | Chloride | Glomerular filtration |
| direct and indirect) | CO2 (bicarbonate) | rate |
| | Creatinine | |

ALP: alkaline phosphatase; ALT: alanine aminotransferase; ANC: absolute neutrophil count; AST: aspartate aminotransferase; BUN: blood urea nitrogen; CRP: C-reactive protein; LDH: lactate dehydrogenase; WBC: white blood cell.

The GFR was calculated using the MDRD formula, as follows:

$$GFR = 175 \times {( serum creatinine )}^{- 1.154} \times {( age )}^{- 0.023}$$

For female subjects, multiply product by 0.742. For African American subjects, multiply product by 1.212.

A full urinalysis and urine protein-to-creatinine ratio was conducted by the study central laboratory (Table 12) as part of the predosing assessment 1 week before each dosing day and on each follow-up safety visit. Subsequently, on each dosing day before dosing and on each safety follow-up visit, urine was assessed for the presence of protein and nitrates as analyzed by the local laboratory (i.e., dipstick analysis).

Any abnormal findings from the local laboratory urine protein-to-creatinine ratio assessments were followed by a confirming evaluation by the study central laboratory. The central laboratory performed urine microscopy if the urinalysis was abnormal. Microscopy consisted of red blood cells/high-power field, white blood cells/high-power field, and casts.

Before each study dose, principal investigators provided the MM with a summary of assessment outcomes associated with the dosing criteria for review and approval. In instances where clinical parameters did not meet dosing criteria, study dosing was temporarily withheld until parameters met dosing levels or discontinued, as outlined in Table 11. Subjects returned for re-evaluation of dosing criteria every 3 weeks until completion of Week 12 of the dosing period, at which time the subject began the safety follow-up period.

TABLE 11
Summary of Dosing Criteria

| | | | |
|---|---|---|---|
| | | Dosing Criteria | |
| Safety Parameter | Continuing Dose | Dose Hold^{a} | Dose Discontinuation^{b} |

Laboratory Investigations

| | | |
|---|---|---|
| Neutrophils | ANC ≥1500/mm³ | Total neutrophils ≥Grade |
| | Thrombocytopenia ≤Grade | 2^{c} |
| | 2 | ANC <1500/mm³ |
| | | Thrombocytopenia |
| | | ≥Grade 3^{c} |
| Platelets | Platelets ≥50,000/mm³ | Platelets <50,000/mm³ |
| T cells | T cells ≥500 cells/μL | |
| | NOTE: Dosing may | |
| | continue in instances of T | |
| | cells <500 cells/μL if | |
| | CD3+ cell count is ≥70% | |
| | of the baseline level | |
| Hgb | ≥8 g/dL | <8 g/dL |
| IgG, IgA, IgM levels | >50% LLN | ≤50% LLN |

Events of Clinical Interest

| | | | |
|---|---|---|---|
| TAK-079 related infusion | <Grade I with symptomatic | | ≥Grade 2 IRR |
| reaction (to include systemic | treatment allowed in | | or CRS^{f} leading to |
| signs and symptoms related to | accordance with Lee et al.^{g} | | moderate clinical |
| CRS^{d}) | NOTE: Dosing may | | symptoms based on a |
| NOTE: IRR is classified in | resume after symptoms | | global assessment of |
| accordance with the NCI | have resolved^{f}. | | symptoms |
| CTCAE criteria, version 4.03. | | | |
| CRS is classified in | | | |
| accordance with Lee et al.^{g} | | | |
| | ≤Grade 2 | | ≥Grade 3^{c} |
| Hypersensitivity (allergic | In accordance with NCI | | In accordance with NCI |
| reaction or anaphylaxis)^{e} | CTCAE. version 4.03 | | CTCAE (version 4.03) |
| | grading for allergic | | grading for allergic |
| | reactions which respond | | reaction or any signs or |
| | promptly to symptomatic | | symptoms of an |
| | measures | | anaphylactic reaction^{h} |
| | | Grade 2^{c} | >Grade 3^{c} |
| Systemic infection | | Hold dosing until the | or infections requiring |
| | | infection is resolved | hospitalization |

ANC: absolute neutrophil count; CRS: cytokine release syndrome; NCI CTCAE: National Cancer Institute Common Terminology Criteria for Adverse Events; Hgb: hemoglobin; IRR: infusion-related reaction; LLN: lower limit of normal; SLE: systemic lupus erythematosus.
^{a}Subjects whose clinical parameters met dose-hold criteria, did not receive their next scheduled cohort dose (i.e., TAK-079 or matching placebo), and instead returned for the next scheduled dose-criteria assessment for reassessment and evaluation of subsequent dosing.
^{b}Subjects whose clinical parameters met dose discontinuation criteria were permanently discontinued from study dosing (i.e., TAK-079 or matching placebo); subjects then advanced to the safety follow-up period of the study, completing all associated assessments, while continuing to receive standard background therapy for SLE, in accordance to principal investigator's discretion and local institutional practices.
^{c}Laboratory grading, allergic reactions, and infection grading was based on NCI CTCAE version 4.03.
^{d}A full cytokine panel was obtained for any suspected events, at any grade, of CRS.
^{e}Further information regarding study-related hypersensitivity reactions is not shown here.
^{f}Further details on the grading and clinical signs and symptoms of CRS is not shown here.
^{g}Lee et al. (2014) Blood 124(2): 188-95; incorporated herein by reference in its entirety.
^{h}Regan and Markowitt (2016) Advancing Transfusion and Cellular Therapies Worldwide Association Bulletin #16-02: 1-4; incorporated herein by reference in its entirety.

TABLE 12
Clinical Urinalysis Assessments

| | Urinalysis |
|---|---|
| Bilirubin | pH |
| Glucose | Protein (including protein-to-creatinine ratio)^{a} |
| Ketones | Specific gravity |
| Leukocytes | Turbidity and color |
| Nitrite^{a} | Urobilinogen |
| Occult blood | |

^{a}Protein and nitrates were assessed as part of the full urinalysis, analyzed by the central laboratory 1 week before each dosing day. In addition, before dosing on study dosing days, protein and nitrates were assessed by local laboratory (i.e., dipstick). Abnormal findings by local laboratory assessment were confirmed by full urinalysis by central laboratory. Further details on assessment timings are provided in Table 5 and Table 6.

### ECGs

Each ECG recording was performed according to standard institutional practice. All study ECGs, including triplicate ECGs, were interpreted by a qualified person (i.e., read locally). Additionally, triplicate 12-lead ECGs were recorded electronically and submitted to a central vendor for storage and for future analysis.

ECGs with clinically significant findings, as judged by the investigator, were considered to be a treatment-emergent adverse event (TEAE); (except for ECGs obtained as part of the screening visit, which were considered part of medical history). Clinically significant findings were recorded on the source documentation and in the eCRF, and underwent continued monitoring.

### AEs

An AE was defined as any untoward medical occurrence in a clinical investigation subject who has signed ICF to participate in a study; it did not necessarily have to have a causal relationship with the treatment. An AE could therefore be any unfavorable and unintended sign (e.g., a clinically significant abnormal laboratory finding), symptom, or disease temporally associated with the use of a drug, whether or not it was considered related to the drug.

Collection of AEs (i.e., AEs, SAEs, AEs of clinical interest, and abnormal liver function tests) commenced at the time the subject signed the ICF. AEs ongoing at end of treatment were monitored until they resolved, returned to baseline, were clearly determined to be due to a subject's stable or chronic condition or intercurrent illness(es), or 6 months after end of treatment had occurred, whichever came first. For subjects who withdrew before the administration of study medication, AEs were followed until the subject discontinued study participation.

All AEs were documented in the AE page of the eCRF.

### Pregnancy

Urine or serum pregnancy testing was performed as outlined in Table 4 and Table 5. Study pregnancy testing could be conducted at a designated local laboratory as determined and confirmed by the sponsor, with appropriate laboratory documentation provided in advance of study testing.

### End-of-Study Assessments

End-of-study clinical parameters, as outlined in Table 13, were assessed on Week 24 (end of safety follow-up period). If clinical presentation and parameters did not meet the end-of-study criteria and were deemed by the principal investigator as study-related, the subject was required to continue on-study, completing a 12-week long-term safety follow-up period, during which time study-related parameters not meeting end-of-study criteria continued to be assessed.

TABLE 13
End-of-Study Clinical Parameters

| Safety Parameter | End-of-Study | |
|---|---|---|
| Laboratory Investigations | Criteria | Continuation to Long-Term Follow-up |
| B cells | ≥LLN or study | <LLN or study baseline levels that is directly related to |
| Neutrophils | baseline levels | dosing of investigational product. |
| Platelets | | |
| T cells | | |
| Hgb | | |
| IgG, IgA, and IgM levels | | |
| Events of clinical interest | | |
| TAK-079-related infusion | ≤Grade 1^{a} | >Grade 2 reaction^{a} CRS leading to moderate clinical |
| reaction (systemic signs and | | symptoms based on a global assessment of symptoms, as |
| symptoms) to include CRS | | classified by the CTCAE version 4.03 (symptoms to include |
| | | but not be limited to headache, fever, chills/rigors, nausea, |
| | | vomiting, diarrhea, arthralgia, myalgia, and hypotension) |
| Systemic infection | | >Grade 2 |
| | | Dosing is to be held until the infection is resolved |

CRS: cytokine release syndrome; CTCAE: Common Terminology Criteria for Adverse Events; Hgb: hemoglobin; LLN: lower limit of normal.
^{a}Grading based on Lee, 2014.

### Appropriateness of Measurement

All of the assessments used in this study are appropriate for the subject population and widely used in studies of subjects with SLE.

### Endpoints

### Primary Endpoint

The primary endpoints evaluated the safety and tolerability of TAK-079, including the incidence, type, and grade of AEs, as well as the percentage of subjects with ≥1 AE leading to study treatment discontinuation.

### Secondary Endpoints

The secondary endpoints included: (a) PK evaluations of TAK-079; (b) PD evaluations of TAK-079; and (c) Immunogenicity assessments of TAK-079.

### Exploratory Endpoints

The exploratory objectives assessed the effects of repeated administration of TAK-079 on disease activity using the following clinical rating scales: (a) CLASI, (b) SLEDAI-2K, (c) PGA of Disease (VAS), and (d) 44-Joint Assessment.

Additionally, biomarkers of disease activity (e.g., anti-dsDNA, anti-ENA antibodies), complement C3 and C4, urine protein-to-creatinine ratio, and/or specific markers of CD38 pathway modulation were also evaluated.

### Drug Concentration Measurement

For drug concentration measurements, see Section of “PK Measurements”.

### Management of Events of Clinical Interest

### Hypersensitivity Reactions: Infusion and Injection Site Reactions

Since infusion reactions and other antibody-mediated hypersensitivity reactions have been reported with other biologic agents, similar AEs may have been seen with TAK-079. Infusion reactions are potentially dose-limiting AEs, but not uncommonly associated with IV administration of biologic agents, but are less frequently associated with SC injection of these therapies.

Symptoms of hypersensitivity may range from mild skin rash to more severe reactions, wheezing, hypotension, poor perfusion, respiratory arrest, and rarely death. Nonanaphylactic clinical hypersensitivity typically occurs within the first hour; however, delayed responses have been reported in the literature. Symptoms of anaphylaxis, a potentially life-threatening condition, range from swelling, angioedema, bronchospasm, respiratory distress, and shock. Hypersensitivity reactions in the literature often occur within a few hours following drug intake. Based on outcomes from studies with daratumumab, patients with pre-existing COPD or asthma may be at particular risk for such respiratory complications as bronchospasm should an infusion reaction event occur. Therefore, patients who forced expiratory volume in 1 second (FEV₁) is <50% of predicted normal were excluded from study participation. Eligible patients with a history of COPD may have required additional postdose medications to manage respiratory complications.

To date, subjects administered TAK-079 have not exhibited anaphylactic symptoms. In the clinical study of healthy subjects (TAK-079-101), an infusion-related reaction (IRR) was defined as a treatment-emergent adverse event (TEAE) occurring within 2 hours of the start of an infusion; there were no IRRs in this study as no allergic or cytokine release reactions were observed within this time period.

### Cytokine Release Syndrome (CRS)

CRS represents an important infusion reaction often associated with the use of monoclonal antibodies used in anti-inflammatory and antitumor therapies. Onset of CRS may occur early in therapy, often after the first infusion of the drug due to a high level of activation of the immune system and engagement and proliferation of T cells that can result in increased cytokine release.

Nonclinical studies have shown that TAK-079 has no agonist activity, suggesting that TAK-079 is unlikely to cause cytokine release due to cell activation. There were no TAK-079-related increases in cytokines at 0.1 mg/kg. At >0.3 mg/kg, dose-related increases (up top 29.5-fold for any individual monkey) in tumor necrosis factor (TNF-α) were observed 30 minutes are the first dose of TAK-079. These increases were considered small compared with elevations observed during a cytokine storm event, were not accompanied by changes in other cytokines, and did not translate into clinical manifestation of an IRR. Increases in serum TNF-α may be related to TAK-079-mediated lyses of CD38+ lymphocytes.

In the FIH study, conducted in healthy subjects, rarely observed symptoms consistent with mild CRS were reported, particularly at higher doses; dose adjustment or interruption was not required.

The CRS hallmark is fever. CRS also presents with rash, urticaria, headache, chills, fatigue, nausea, and/or vomiting. Severe CRS is characterized by severe dyspnea, often accompanied by bronchospasm and hypoxia, in addition to fever, chills, rigors, urticaria, and angioedema. The acute respiratory failure may be accompanied by such events as pulmonary interstitial infiltration or edema visible on a chest x-ray. The syndrome frequently manifests within 1 or 2 hours of initiating the first infusion. Patients with a history of pulmonary insufficiency or those with pulmonary tumor infiltration may be at greater risk of poor outcome and should be treated with increased caution. On the basis of outcomes from the studies of the anti-CD38 mAb daratumumab, patients with pre-existing COPD or asthma may be at particular risk for respiratory complications, such as bronchospasm, should an infusion reaction or CRS event occur.

Therefore, if the patient's FEV₁ is <50% of predicted normal, they were excluded from study participation. Eligible patients with a history of COPD may require additional postinfusion medications to manage respiratory complications.

### Hematologic Effects

Reductions in platelets, lymphocytes, and red blood cells (RBCs) occurred in nonclinical studies in some animals administered doses of TAK-079 higher than the no-observed-adverse-effect level (NOAEL) of 0.3 mg/kg. In the FIH study, no decreases were seen in RBCs or platelets, despite observations of reductions in these cell counts in monkey toxicology studies after repeated dosing at higher dose levels (≥1 mg/kg).

Patients were monitored closely, including testing of hematology parameters throughout this clinical study. In instances where clinical parameters did not meet dosing criteria, study dosing was temporarily or permanently withheld. Medical interventions were administered according to institutional guidelines.

### Infections

In a Good Laboratory Practice (GLP)-compliant 13-week toxicology study, bacterial and/or viral infection, secondary to immune suppression, was observed in cynomolgus monkeys at IV doses of 3, 30, and 80 mg/kg administered once every 2 weeks. The NOAEL dose of 0.3 mg/kg, administered IV once every week, was not associated with infections. In the FIH study, mild infections, specifically nasopharyngitis, was reported.

Patients were monitored for any signs and symptoms of infections throughout this clinical study. In instances where clinical parameters did not meet dosing criteria, study dosing was temporarily or permanently withheld. Management of infections according to standard medical care was recommended.

### Antidrug Antibody Interactions

Antidrug antibody (ADA) responses were detected in most monkeys in the single-dose PK studies and the 4-week (non-GLP) and 13-week (GLP) toxicology studies. Stronger positive ADA responses were generally associated with lower serum concentrations of TAK-09, and this was especially notable in the 13-week repeat-dose toxicity studies and at lower doses. In the single-dose healthy subject study (TAK-079-1001), 5 of 54 TAK-079-treated subjects were positive for ADA (3 subjects with transient ADA and 2 subjects with persistent ADA). Of these, one subject was treated with the 0.06 mg IV cohort and the remaining 4 subjects were treated with 0.03 mg/kg (2 subjects), 0.1 mg/kg (1 subject) or 0.6 mg/kg (1 subject) SC TAK-079. Immunogenicity was not associated with clinically significant AEs, even in the 2 subjects with persistent immunogenicity.

### Overdose

An overdose is defined as a known deliberate or accidental administration of investigational drug, to or by the study subject, at a dose above that which was assigned to that individual subject according to the study protocol.

To date, there is no experience with overdose. If an overdose had occurred, close monitoring and supportive treatment, as medically required, was recommended.

### Statistical Methods and Determination of Sample Size

### Analysis Sets

Safety Analysis Set: The safety analysis set included all subjects who were enrolled and received at least 1 dose of study drug. This analysis set was used for demographic, baseline characteristics, efficacy, and safety summaries.

PK Analysis Set: The PK analysis set included all subjects who received study drug and had at least 1 measurable serum concentration.

PD Analysis Set: The PD analysis set included all subjects who received study drug and had at least 1 postdose PD measurement.

Immunogenicity Analysis Set: The immunogenicity analysis set included all subjects from the safety population who had the baseline immunogenicity sample and at least 1 postdose immunogenicity sample assessment.

### General Considerations

Continuous data was summarized using the following descriptive statistics: number of subjects, mean, SD, median, minimum, and maximum, where appropriate. The percent coefficient of variation (% CV) and geometric mean was included in the summary of continuous data where indicated. Arithmetic means, geometric means, and medians were presented to 1 more decimal place than the recorded data and SDs were presented to 2 more decimal places than the recorded data, where appropriate.

For PK related tables, mean, SD, median, minimum, and maximum were rounded to 3 significant digits and % CV was presented to 1 decimal place.

Categorical data was summarized using the number and percent of subjects for each category, where appropriate. Percentages were reported to 1 decimal place.

Unless otherwise stated, baseline value was defined as the last observed value before the first dose of study medication.

There were no visit windows for the dosing visits, safety follow-up period, and long-term safety follow-up period. All non-dosing visits during the dosing period had a window of +/−1 day.

For values with several non-missing measurements, for example triplicate ECGs, the average of triplicate assessments was used.

Subjects who received placebo in each cohort were pooled across the cohorts. All summaries were presented by the pooled placebo group, TAK-079 doses, TAK-079 overall subjects in the study separately.

For analyses based on the safety, PK, PD, or immunogenicity analysis sets, subjects were analyzed as treated, that is, actual treatment. For analyses based on all randomized subjects, subjects were analyzed as randomized, that is, planned treatment.

### Disposition of Subjects

The summary and listing of disposition of subjects was based on all randomized subjects. The number of subjects in the safety analysis set was presented. Disposition was tabulated in terms of subjects who prematurely discontinued from study treatment, subjects who completed study treatment, subjects who prematurely discontinued from the study, and subjects who completed the study. Primary reasons for premature discontinuation from study or study treatment, as entered on the eCRF, were tabulated. Percentages were based on the number of subjects in the safety analysis set.

In addition, the number of subjects randomized was summarized for each country and site.

Major protocol deviations were summarized and listed based on all randomized subjects. Protocol deviations related to coronavirus disease 2019 (COVID-19) were summarized.

The date of first dose, date of last dose, duration of treatment and the reason for premature discontinuation of study drug/study visit were presented for each subject in listings.

### Demographic and Other Baseline Characteristics

### Demographics

Summary statistics were presented for continuous variables (for example, age and weight). The number and percentage subjects within each category were presented for categorical variables (for example, race and sex). Individual subject demographic and baseline characteristic data were listed. Placebo data was pooled across the cohorts.

Demographic variables of screen failure subjects and reasons for screen failures were summarized for subjects who were screened, but not enrolled in the study.

Individual demographic characteristics, date of ICF, and reason for screen failure were listed.

The analysis and listing of demographics and baseline characteristics were based on the safety analysis set.

### Medical History

Medical history refers to any significant conditions or diseases that stopped at or before informed consent or are ongoing at informed consent.

Medical history was coded using the Medical Dictionary for Regulatory Activities (MedDRA, version 24.0) and was summarized by treatment using System Organ Class (SOC) and MedDRA Preferred Term (PT). The table includes number and percentages of subjects, and is sorted in alphabetical order by SOC. Within an SOC, PTs were sorted in decreasing frequency based on the total number of subjects. A subject was only counted once within a particular class even if he/she had multiple conditions/symptoms. Summaries were based on the safety analysis set.

All medical history data was presented in listings based on the safety analysis set.

### Prior and Concomitant Medications

Concomitant medications, blood products, and procedures were recorded from the first dose of TAK-079 or matching placebo through the end of the safety follow-up period (i.e., end of safety follow-up visit in Week 24 or, when applicable, end of long-term safety follow-up visit in Week 36). Trade name and international nonproprietary name (if available), indication, and start and end dates of the administered medication were recorded. Medications were coded using the World Health Organization Drug Dictionary (B3 Global version SEP2018 or later). The number and percentage of subjects taking prior medications and concomitant medications was tabulated by World Health Organization Drug Dictionary generic term based on safety analysis set.

All prior medications and concomitant medications data were presented in a listing based on the safety analysis set. There was no imputation for missing concomitant medication dates.

### Efficacy Analysis

Exploratory efficacy endpoints were summarized by descriptive statistics. Additionally, where applicable, analyses of treatment effects were presented in terms of point estimates and 2-sided 95% CIs. The safety analysis set was used for all efficacy analyses.

Rating scales were also analyzed using longitudinal modeling and responder analysis.

### Longitudinal Modeling

Change from baseline at each scheduled visit were calculated for the CLASI, SLEDAI-2K, PGA of Disease (VAS), and 44-Joint Assessment assessments.

Analyses of rating scale change from baseline by visit was performed using a mixed model for repeated measures (MMRM) analysis, including treatment, visit, and (treatment x visit) interaction terms as the factors, with baseline values as covariates, and further adjusted by baseline-by-visit interaction. The unstructured covariance was used as the default structure for the model. Alternative covariance structures were evaluated if there were convergence issues. Comparisons of mean changes from baseline between different doses of TAK-079 and placebo were performed for all scheduled postbaseline assessment time points. CIs based on least squared means for the differences between groups were presented. Based on a “missing at random” assumption, this analysis was performed using observed case data only.

Observed rating scale values and change from baseline by visit were summarized descriptively. The observed and MMRM model based mean changes from baseline for each treatment group were plotted. The rating scales were provided in the by-subject listing. Individual subject profiles over time for selected rating scales or sub-scales may have been plotted as needed.

### Responder Analysis

The percentage of subjects meeting the following clinical response criteria for each clinical rating scales were calculated for each scheduled assessment:

CLASI total activity score: score decreased from baseline either by ≥4 points or by ≥20% (only subjects who had a baseline score >0 were analyzed).

SLEDAI-2K total score: score decreased from baseline by ≥4 points.

PGA of Disease (VAS): score decreased from baseline by ≥0.3 points.

Subjects meeting the criteria for clinical response in each clinical rating scale were considered as responders on that clinical rating scale. Comparisons of responder rates between different doses of TAK-079 and placebo were performed for all scheduled postbaseline assessment time points. CIs for differences of proportions were presented. This analysis was based only on observed data at each time point (i.e., complete case analysis). Missing data was not imputed.

The observed percentage of responders were summarized descriptively by visit. 95% CIs for percentage of responders by treatment group may have been produced. The observed percentage of responders over time may have been plotted.

### Safety Analysis

### AEs

TEAEs were defined as AEs that occurred after the first dose of study drug received in the treatment period and until the end of safety follow-up.

TEAEs were presented by intensity. Treatment-emergent SAEs, TEAEs leading to study drug discontinuation, TEAEs leading to dose modification (e.g., described as dose delay, dose skipped) and TEAEs leading to death were also summarized using SOC and PT.

When calculating the frequency and percentage of subjects who reported TEAEs, a subject was counted only once for each SOC or PT when multiple TEAEs were coded to the same SOC or PT. For the intensity or relatedness summaries, if a subject reported multiple TEAEs coded to the same SOC or PT, the TEAE with maximum intensity or strongest relationship was included.

AEs with missing intensity were listed as such in the AE listings, but were summarized as severe in summary tables. Similarly, if the relationship of an event was missing, the event was considered as related but in listings it was presented as missing.

There was no imputation for missing AEs dates.

### Clinical Laboratory Evaluations

If a subject had repeated laboratory values for a given time point, the value from the last evaluation was used.

All laboratory test parameters were displayed in individual subject data listings in both Système Internationale (SI) units and conventional (CV) units. For test results not in SI units, the conversion to SI units used the known conversion factors. If necessary, SI units from the central laboratory may have been converted to Takeda's preferred SI units. All summaries and analyses were based on the values using preferred SI units; data was presented in both SI and conventional units in data listings.

Common Terminology Criteria for Adverse Events (CTCAE) grading (version 4.03) for laboratory values were summarized by creating a shift table for the toxicity grades for baseline to worst toxicity grade postbaseline.

### Vital Signs

Individual results of vital signs that met abnormal criteria and were clinically significant per the PI were captured as an AE by the investigator.

### 12-Lead ECGs

Overall ECG interpretation category (normal, abnormal not clinically significant, abnormal clinically significant, not evaluable) was collected by eCRF at baseline and at each scheduled postbaseline visit. Shifts in ECG interpretation were presented as cross-tabulations (baseline versus each postbaseline visit) of numbers of subjects with normal, abnormal not clinically significant, and abnormal clinically significant interpretations, not evaluable, with missing, if applicable, and total categories overall.

Rate-corrected QT intervals (milliseconds) of electrocardiograph (corrected QT [QTc]) were calculated using Bazett correction and Fridericia correction, if necessary. The formulas were:

$$QTc ( Bazett ) = QT / ( RR \hat{} 0.5 )$$

$$QTc ( Fridericia ) = QT / ( RR \hat{} 0.33 )$$

where RR=60/heart rate (beats per minute). All ECG parameters were listed in a data listing.

### Extent of Exposure and Compliance

The total number of doses taken and the total amount of dose taken were summarized descriptively in the pooled placebo group and TAK-079 arms.

Treatment compliance was summarized in terms of the percent of scheduled doses received in the pooled placebo group and TAK-079 arms. The percent of scheduled doses received for each subject was defined as: [(actual total number of doses taken)/(planned number of doses)]×100.

The date and time of each dose for each subject was reported in a data listing.

### PK, PD, and Biomarker Analyses

### PD and Biomarker Analysis

Individual values, change from baseline, and mean change from baseline and/or percent change from baseline at different time points for placebo and each TAK-079 dose level were calculated for various immune cell subsets including plasma cells, PBs, NK cells, B cells, T cells, monocytes, and total lymphocytes. CD38 expression and receptor occupancy for plasma cells, PBs, NK cells, B cells, T cells, and monocytes were also evaluated. Cytokine measurements and change from baseline at different timepoints for placebo and each TAK-079 dose level were calculated for each subject. Individual values and changes in the levels of autoantibodies, immunoglobulin and complement proteins were summarized.

Descriptive summaries included geometric mean and % CV where needed.

Figures of mean values and mean percent change from baseline were generated. Mean percent change from baseline was not plotted for measures related to receptor occupancy, instead mean change from baseline was generated. Individual subject profiles over time for selected parameters may have been plotted as needed.

### PK Analysis

Types of PK Analysis and Methods: Concentrations of TAK-079 in serum and whole blood summarized by TAK-079 dose level at each scheduled sampling time using descriptive statistics (N, arithmetic mean, SD, median, maximum, and percent coefficient of variation [% CV]). Individual plasma concentration data versus time presented in a data listing. PK parameters of TAK-079 summarized for each dose level using descriptive statistics. Geometric mean computed for maximum observed concentration (Cₘₐₓ) and areas under the concentration-time curve (AUCs). Dose proportionality assessed graphically and using the power model. Plots of Cₘₐₓ and AUCs, as well as dose-normalized Cₘₐₓ and AUCs, versus doses. Other analyses or methods are contemplated, if appropriate.

Due to sparse PK sampling, no noncompartmental analysis was conducted. TAK-079 concentration-time profile was summarized using descriptive statistics. Individual TAK-079 concentration-time data, individual data on the last concentration reached before the next dose administration (C_{trough}) and individual maximum observed concentration (Cₘₐₓ) were presented in listings and tabulated using summary statistics by treatment group. Individual and mean concentration-time profiles were plotted by treatment group. The PK analysis set was used for summaries and analyses of PK parameters.

A population PK model may be developed. If developed, the population PK model will be reported separately. The analysis plan for the population PK analysis will be separately defined, and the results of these analyses will be reported separately.

The PK-PD relationship may be explored graphically. PK/PD models may be developed to explore the relationship between TAK-079 serum concentrations and cell counts (e.g., plasma cells, PBs, NK cells, B cells, T cells, monocytes, and total lymphocytes). If developed, the results will be reported in a separate report. PD parameters will also be listed in data listings. Additional PD analysis to evaluate the dose effect may be performed, if appropriate.

### Immunogenicity Analysis

Immunogenicity analysis (ADA transient, persistent, negative, and titer) was summarized using descriptive statistics as applicable. The relationship between immunogenicity responses and efficacy and safety may be explored.

Transiently positive ADA response was defined in the SAP as subjects who had confirmed positive ADA status in 1 or 2 postbaseline assessment(s).

Persistently positive ADA response was defined in the SAP as subjects who have confirmed positive ADA status in >2 postbaseline assessments.

A positive ADA assessment at each postbaseline visit was defined in the SAP as having either (1) a negative assessment at baseline and a positive assessment value, or (2) a positive value at baseline and an assessment value that met the criterion for treatment-boosted ADA response (>4 times the baseline value).

During the actual analysis of study data, changes and additions were made to the SAP-defined ADA responses outlined above. These changes are described in detail in Section of “Changes to Immunogenicity Analysis”.

### Interim Analyses

The sponsor may have performed unblinded reviews or analyses of data from 1 or more dose cohorts after all subjects enrolled in those cohorts completed the safety follow-up period or prematurely discontinued from the study. The sponsor received treatment assignments only for individual subjects within the pertinent cohorts while remaining blinded to treatment assignments of the other subjects.

### Determination of Sample Size

This study was not statistically powered for any hypothesis testing. The sample size of 6 active and 2 placebo subjects for each of the 3 cohorts (resulting in 6 subjects treated with placebo or with 45 mg, 90 mg, or 135 mg TAK-079) were considered to be sufficient to fulfill the study objectives of the evaluation of safety, tolerability, and PK of each cohort.

### Example 2: Study Subjects

### Disposition of Subjects

In total, 35 subjects were screened but did not enter the study. Reasons for screen failure were not meeting the entrance criteria (28 subjects), withdrawal by subject (5 subjects), and other (2 subjects).

The study planned to randomize 24 subjects; however, enrollment was closed early due to a high study screen failure rate and operational challenges with subject recruitment.

Twenty-three subjects were randomized at 13 study sites in the United States. One randomized subject discontinued the study before receiving study drug and 22 randomized subjects received at least 1 dose of study drug and were included in the safety analysis set (Table 14).

The mean age was lower in the placebo group (36.4 years) than in the mezagitamab groups (pooled mezagitamab group: 49.1 years). The mean baseline weight was higher in the placebo (87.3 kg) and mezagitamab 90 mg (85.6 kg) groups and lower in the mezagitamab 45 mg (75.0 kg) and mezagitamab 135 mg (64.6 kg) groups. Other demographic variables were similar across treatment groups. Most study subjects were female and there were nearly equal percentages of African American and White study subjects.

TABLE 14
Subject Disposition Summary (All Randomized Subjects)

| | Number (%) | | | | | | | | |
|---|---|---|---|---|---|---|---|---|---|
| | Pooled | TAK-079 | | TAK-079 | TAK-079 | Total | | | |
| | Placebo | 45 mg | | 90 mg | 135 mg | TAK-079 | | Overall | |
| | (n = 6) | (n = 6) | | (n = 6) | (n = 6) | (n = 17) | | (n = 23) | |
| Safety analysis set^{a} | 5 | 6 | | 6 | 5 | 17 | | 22 | |
| Discontinued study | 0 | 0 | | 4 (66.7) | 2 (40.0) | 6 | (35.3) | 6 | (27.3) |
| treatment prematurely | | | | | | | | | |
| Completed study treatment | 5 (100.00) | 6 | (100.00) | 2 (33.3) | 3 (60.00) | 11 | (64.7) | 16 | (72.7) |
| Primary reason discontinued | | | | | | | | | |
| study treatment prematurely | | | | | | | | | |
| AE | 0 | 0 | | 0 | 1 (20.0) | 1 | (5.9) | 1 | (4.5) |
| Protocol deviation | 0 | 0 | | 0 | 0 | 0 | | 0 | |
| Lost to follow-up | 0 | 0 | | 0 | 0 | 0 | | 0 | |
| Withdrawal by subject | 0 | 0 | | 1 (16.7) | 1 (20.0) | 2 | (11.8) | 2 | (9.1) |
| Termination of study by | 0 | 0 | | 0 | 0 | 0 | | 0 | |
| sponsor | | | | | | | | | |
| Other | 0 | 0 | | 3 (50.0) | 0 | 3 | (17.6) | 3 | (13.6) |
| Discontinued study | 0 | 1 | (16.7) | 1 (16.7 | 2 (40.0) | 4 | (23.5) | 4 | (18.2) |
| prematurely^{b} | | | | | | | | | |
| Completed study^{b} | 5 (100.00) | 5 | (83.3) | 5 (83.3) | 3 (60.0) | 13 | (76.5) | 18 | (18.8) |
| Primary reason discontinued | | | | | | | | | |
| study prematurely | | | | | | | | | |
| Death | 0 | 0 | | 0 | 0 | 0 | | 0 | |
| AE | 0 | 0 | | 0 | 0 | 0 | | 0 | |
| Protocol deviation | 0 | 0 | | 0 | 0 | 0 | | 0 | |
| Lost to follow-up | 0 | 0 | | 0 | 0 | 0 | | 0 | |
| Withdrawal by subject | 0 | 1 | (16.7) | 1 (16.7) | 2 (40.0) | 4 | (23.5) | 4 | (18.2) |

| | | | | | | |
|---|---|---|---|---|---|---|
| Lack | 0 | 0 | 0 | 0 | 0 | 0 |
| Termination of study by | 0 | 0 | 0 | 0 | 0 | 0 |
| sponsor | | | | | | |
| Other | 0 | 0 | 0 | 0 | 0 | 0 |

AE: adverse event.
^{a}The safety analysis set consisted of all subjects who were enrolled a received at least 1 dose of study drug.
^{b}A subject who completed the Week 24 visit was considered to have completed the study. All other subjects were considered to have discontinued the study prematurely.

### Protocol Deviations

In total, 3 subjects reported major protocol deviations, 1 under the category of informed consent and 2 under the category of study treatment administration/dispensing.

### Impact of the Covid-19 Pandemic on Study Subject

In total, 2 subjects reported protocol deviations related to COVID-19. A subject in the TAK-079 90 mg group reported deviations in the 2 categories of study procedure/assessments and visit scheduling, and a subject in the TAK-079 135 mg group reported deviations in the 2 categories of study treatment compliance and visit scheduling.

### Example 3: Pk, Pd, Immunology, and Efficacy Evaluations Data Sets Analyzed

The number of subjects in each analysis set is shown in Table 15. The safety analysis set was used for demographic, baseline characteristics, efficacy, and safety summaries.

TABLE 15
Summary of Analysis Sets (All Randomized Subjects)

| | Number (%) | | | | | | | | | | | |
|---|---|---|---|---|---|---|---|---|---|---|---|---|
| | Pooled | | TAK-079 | | TAK-079 | | TAK-079 | | Total | | | |
| | Placebo | | 45 mg | | 90 mg | | 135 mg | | TAK-079 | | Overall | |
| Started | 6 | (100.0) | 6 | (100.0) | 6 | (100.0) | 5 | (100.0) | 17 | (100.0) | 23 | (100.0) |
| Safety set^{a} | 5 | (83.3) | 6 | (100.0) | 6 | (100.0) | 5 | (100.0) | 17 | (100.0) | 22 | (95.7) |
| PK set^{b} | 0 | | 6 | (100.0) | 6 | (100.0) | 5 | (100.0) | 17 | (100.0) | 17 | (73.9) |
| PD set^{c} | 5 | (83.3) | 6 | (100.0) | 6 | (100.0) | 5 | (100.0) | 17 | (100.0) | 22 | (95.7) |
| Immunogenicity set^{d} | 5 | (83.3) | 6 | (100.0) | 5 | (83.3) | 5 | (100.0) | 16 | (94.1) | 21 | (91.3) |
| Completed | 5 | (83.3) | 5 | (83.3) | 5 | (83.3) | 3 | (60.0) | 13 | (76.5) | 18 | (78.3) |

| | | | | | | | | | | | |
|---|---|---|---|---|---|---|---|---|---|---|---|
| Withdrawal by subject | 0 | 1 | (16.7) | 1 | (16.7) | 2 | (40.0) | 4 | (23.5) | 4 | (17.4) |

| | | | | | | | | |
|---|---|---|---|---|---|---|---|---|
| Randomized but not treated | 1 | (16.7) | 0 | 0 | 0 | 0 | 1 | (0.04) |

PK: pharmacokinetic; PD: pharmacodynamic.
Percentages are based on all subjects who were randomized. One subject was randomized but did not receive any dose.
^{a}The safety analysis set consisted of all subjects who were enrolled and received at least 1 dose of study drug.
^{b}The PK set consisted of all subjects who received study drug and had at least 1 measurable serum concentration.
^{c}The PD set consisted of all subjects who received study drug and had at least 1 postdose PD measurement.
^{d}The immunogenicity set consisted of all subjects who received study drug and had a baseline and at least 1 postbaseline immunogenicity sample assessment.

### Demographic Characteristics

Demographic characteristics are summarized in Table 16.

The mean age was lower in the placebo group (36.4 years) than in the TAK-079 groups (pooled TAK-079 group: 49.1 years). Other demographic variables were similar across treatment groups. Most study subjects were female and there were nearly equal percentages of African American and White study subjects.

TABLE 16
Demographics (Safety Analysis Set)

| | Pooled | | TAK-079 | | TAK-079 | | TAK-079 | | Total | | | |
|---|---|---|---|---|---|---|---|---|---|---|---|---|
| | Placebo | | 45 mg | | 90 mg | | 135 mg | | TAK-079 | | Overall | |
| | (N = 5) | | (N = 6) | | (N = 6) | | (N = 5) | | (N = 17) | | (N = 22) | |
| Age (years) | | | | | | | | | | | | |
| n | 5 | | 6 | | 6 | | 5 | | 17 | | 22 | |
| Mean (SD) | 36.4 | (6.58) | 51.0 | (22.01) | 46.7 | (6.53) | 49.6 | (13.50) | 49.1 | (14.63) | 46.2 | (14.17) |
| Median | 36.0 | | 62.5 | | 46.5 | | 56.0 | | 50.0 | | 44.0 | |
| Min, max | 26, 43 | | 19, 70 | | 39, 57 | | 31, 63 | | 19, 70 | | 19, 70 | |
| Sex, n(%) | | | | | | | | | | | | |
| Male | 0 | | 0 | | 1 | (16.7) | 1 | (20.0) | 2 | (11.8) | 2 | (9.1) |
| Female | 5 | (100.0) | 6 | (100.0) | 5 | (83.3) | 4 | (80.0) | 15 | (88.2) | 20 | (90.9) |
| Ethnicity, n(%) | | | | | | | | | | | | |
| Hispanic or Latino | 1 | (20.0) | 2 | (33.3) | 0 | | 1 | (20.0) | 3 | (17.6) | 4 | (18.2) |
| Non-Hispanic and | 4 | (80.0) | 4 | (66.7) | 6 | (100.0) | 4 | (80.0) | 14 | (82.4) | 18 | (81.8) |
| Latino | | | | | | | | | | | | |
| Not reported | 0 | | 0 | | 0 | | 0 | | 0 | | 0 | |
| Unknown | 0 | | 0 | | 0 | | 0 | | 0 | | 0 | |
| Racc, n(%)^{a} | | | | | | | | | | | | |
| American Indian or | 0 | | 0 | | 0 | | 1 | (20.0) | 1 | (5.9) | 1 | (4.5) |
| Alaska Native | | | | | | | | | | | | |
| Asian | 0 | | 0 | | 0 | | 1 | (20.0) | 1 | (5.9) | 1 | (4.5) |
| Black or African | 2 | (40.0) | 4 | (66.7) | 1 | (16.7) | 2 | (40.0) | 7 | (41.2) | 9 | (40.9) |
| American | | | | | | | | | | | | |
| Native Hawaiian or | 0 | | 0 | | 0 | | 0 | | 0 | | 0 | |
| other Pacific Islander | | | | | | | | | | | | |
| White | 3 | (60.0) | 2 | (33.3) | 4 | (66.7) | 1 | (20.0) | 7 | (41.2) | 10 | (45.5) |
| Not reported | 0 | | 0 | | 0 | | 0 | | 0 | | 0 | |
| Multiracial | 0 | | 0 | | 1 | (16.7) | 0 | | 1 | (5.9) | 1 | (4.5) |
| Baseline weight (kg) | | | | | | | | | | | | |
| n | 5 | | 6 | | 6 | | 5 | | 17 | | 22 | |
| Mean (SD) | 87.3 | (17.7) | 75.0 | (18.4) | 85.6 | (28.4) | 64.6 | (8.16) | 75.7 | (21.2) | 78.3 | (20.7) |

| | | | | | | |
|---|---|---|---|---|---|---|
| Median | 89.400 | 82.500 | 74.810 | 62.700 | 74.620 | 74.810 |
| Min, max | 68.40, 112.26 | 49.44, 90.30 | 65.41, 141.07 | 55.57, 77.02 | 49.44, 141.07 | 49.44, 141.07 |

Max: maximum; min: minimum.
^{a}A subject may have more than 1 race. Those subjects selecting multiple races are counted only under “multiracial”.

### Medical History and Other Baseline Characteristics

All 22 (100%) subjects had any reported medical history. Common medical history excluding SLE (≥25% of total subjects) included gastrooesophageal reflux disease (13 subjects [59.1%]), hypertension (11 subjects [50.0%]), depression (10 subjects [45.5%]), anxiety (9 subjects [40.9%]), hyperthyroidism (7 subjects [31.8%]), and migraine, insomnia, and vitamin D deficiency (6 subjects [27.3%] each).

### Medication History and Concomitant Medications

### Medication History

All 22 (100%) subjects had at least 1 prior medication. Common prior medications (≥25% of total subjects) included hydroxychloroquine (13 subjects, 59.1%), prednisone (9 subjects, 40.9%), gabapentin (8 subjects, 36.4%), and hydroxychloroquine sulfate (6 subjects, 27.3%).

### Concomitant Medications

All 22 (100%) subjects had at least 1 concomitant medication, reflecting the fact that all subjects were required to receive stable background SLE therapy throughout the study. The most common background SLE medications in this study included the following:

Antimalarials: pooled placebo, 4 subjects (80.0%); TAK-079 45 mg, 5 subjects (83.3%); TAK-079 90 mg, 6 subjects (100%); TAK-079 135 mg, 4 subjects (80.0%).

Hydroxychloroquine (13 subjects, 59.1%): pooled placebo, 3 subjects (60.0%); TAK-079 45 mg, 3 subjects (50.0%); TAK-079 90 mg, 4 subjects (66.7%); TAK-079 135 mg, 3 subjects (60.0%). Hydroxychloroquine sulfate (6 subjects, 27.3%): pooled placebo, 1 subject (20.0%); TAK-079 45 mg, 2 subjects (33.3%); TAK-079 90 mg, 2 subjects (33.3%); TAK-079 135 mg, 1 subject (20.0%).

Corticosteroids [mean dose, mg]: pooled placebo, 3 subjects (60.0%) [8.3 mg]; TAK-079 45 mg, 2 subjects (33.3%) [10 mg]; TAK-079 90 mg, 2 subject (33.3%) [7 mg]; TAK-079 135 mg, 3 subjects (60.0%) [4.6 mg]. Prednisone (9 subjects, 40.9%): pooled placebo, 3 subjects (60.0%); TAK-079 45 mg, 2 subjects (33.3%); TAK-079 90 mg, 1 subject (16.7%); TAK-079 135 mg, 2 subjects (60.0%). Methylprednisolone (1 subject, 4.5%): pooled placebo, 0 subjects; TAK-079 45 mg, 0 subjects; TAK-079 90 mg, 1 subject (16.7%); TAK-079 135 mg, 0 subjects.

Gabapentin (8 subjects, 36.4%): pooled placebo, 2 subjects (40%); TAK-079 45 mg, 3 subjects (50%); TAK-079 90 mg, 1 subject (16.7%); TAK-079 135 mg, 2 subjects (40%).

Mycophenolate: pooled placebo, 1 subject (20.0%); TAK-079 45 mg, 2 subjects (33%); TAK-079 90 mg, 1 subject (16.7%); TAK-079 135 mg, 3 subjects (60.0%). Mycophenolate mofetil: pooled placebo, 1 subject (20.0%); TAK-079 45 mg, 1 subject (16.7%); TAK-079 90 mg, 0 subjects; TAK-079 135 mg, 2 subjects (40.0%). Mycophenolate sodium: pooled placebo, 0 subjects; TAK-079 45 mg, 0 subjects; TAK-079 90 mg, 1 subject (16.7%); TAK-079 135 mg, 0 subjects. Mycophenolic acid: pooled placebo, 0 subjects; TAK-079 45 mg, 1 subject (16.7%); TAK-079 90 mg, 0 subjects; TAK-079 135 mg, 1 subject (20.0%).

Methotrexate: pooled placebo, 1 subject (20.0%); TAK-079 45 mg, 2 subjects (33.3%); TAK-079 90 mg, 0 subjects; TAK-079 135 mg, 0 subjects.

Overall, background medications were balanced across treatment groups and were not changed during the treatment period. Among those subjects receiving corticosteroids (prednisone and methylprednisolone), slightly higher mean daily doses were observed in the placebo (8.3 mg) and TAK-079 45 mg (10.0 mg) groups compared with the TAK-079 90 mg (7.0 mg) and 135 mg (4.7 mg) groups during the treatment period.

### Measurements of Treatment Compliance

Study drug compliance data is provided in Table 17.

TABLE 17
Study Drug Exposure and Compliance (Safety Analysis Set)

| | Pooled | TAK-079 | TAK-079 | TAK-079 | Total |
|---|---|---|---|---|---|
| | Placebo | 45 mg | 90 mg | 135 mg | TAK-079 |
| | (N = 5) | (N = 6) | (N = 6) | (N = 5) | (N = 17) |
| Study drug exposure, months | | | | | |
| Mean (SD) | 3.2 (0.84) | 3.2 (0.75) | 2.5 (1.38) | 2.8 (0.84) | 2.8 (1.01) |
| Median | 3.0 | 3.0 | 2.5 | 3.0 | 3.0 |
| Min, max | 2, 4 | 2, 4 | 1, 4 | 2, 4 | 1, 4 |
| Total amount of dose taken (mg) | | | | | |
| Mean (SD) | 0.0 (0.00) | 142.5 (33.87) | 225.0 (124.06) | 378.0 (112.95) | 240.9 (134.00) |
| Median | 0.0 | 135.0 | 225.0 | 405.0 | 180.0 |
| Min, max | 0, 0 | 90, 180 | 90, 360 | 270, 540 | 90, 540 |
| Treatment compliance (%)^{a} | | | | | |
| Mean (SD) | 80.0 (20.92) | 79.2 (18.82) | 62.5 (34.46) | 70.0 (20.92) | 70.6 (25.36) |
| Median | 75.0 | 75.0 | 62.5 | 75.0 | 75.0 |
| Min, max | 50, 100 | 50, 100 | 25, 100 | 50, 100 | 25, 100 |

^{a}Treatment compliance (%) was calculated as (actual number of doses taken)/(planned number of doses) × 100

### Analysis of Pk, Pd, Immunology, and Efficacy Results

### PK Results—Serum Concentrations of TAK-079

Serum concentrations of TAK-079 were detectable in all subjects at all dose levels. However, TAK-079 concentrations were below the lower limit of quantitation postdose in a number of subjects, particularly in 45 mg dose group. Serum PK data excluded from PK analysis and or/reporting were due to dose holds and premature discontinuations (Section of “Extent of Exposure”), as well as other reasons related to sample availability and condition.

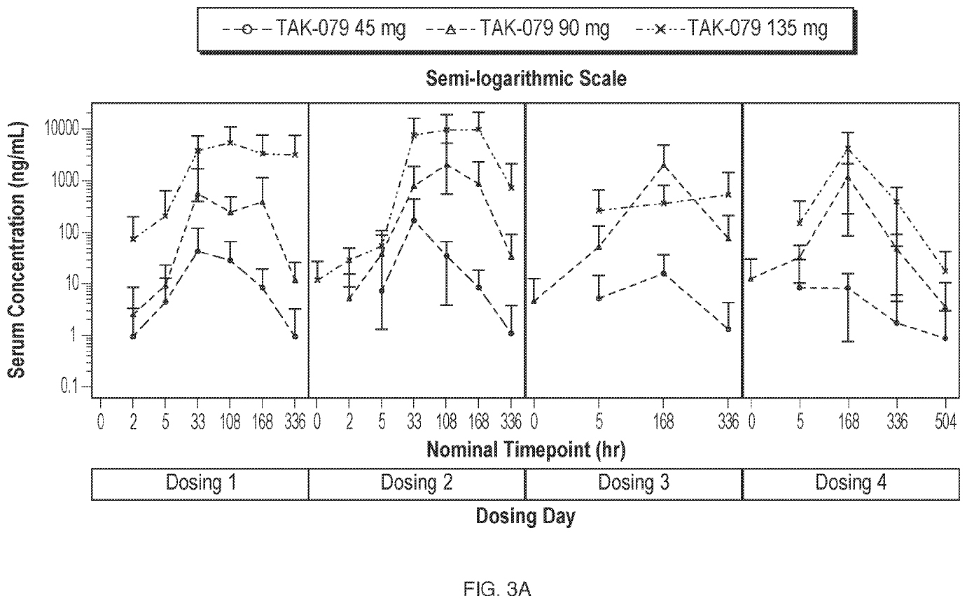

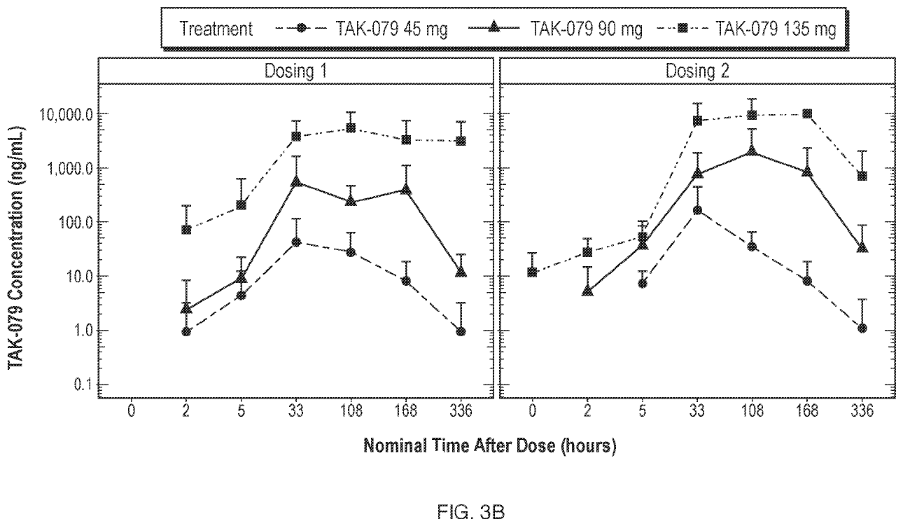

Log-linear plot of mean/SD serum concentrations of TAK-079 versus time following multiple dose administration of TAK-079 with SC injection at 45, 90, and 135 mg are presented in FIG. 3A and FIG. 3B. Cₘₐₓ maximum observed plasma concentration and number of participants with a change from the baseline in CD38 expression level and receptor occupancy of plasma cells, plasmablasts, NK cells, B cells, T cells, and monocytes are presented in Tables 18 and 19, respectively.

TABLE 18
Cₘₐₓ Maximum Observed Plasma Concentration for TAK-079 (PK analysis set).

| | | TAK-079 | TAK-079 | TAK-079 |
|---|---|---|---|---|
| | | 45 mg | 90 mg | 135 mg |
| Overall Number of Participants Analyzed^{a} | | 6 | 6 | 5 |
| Day 1 | Number Analyzed^{b} | 6 | 6 | 5 |
| | Mean (SD) (ng/mL) | 57.5 (71.7) | 660.0 (1050) | 6130 (5170) |
| Day 22 | Number Analyzed | 6 | 5 | 5 |
| | Mean (SD) (ng/mL) | 95.4 (187) | 1570 (2930) | 6330 (9450) |
| Day 43 | Number Analyzed | 6 | 4 | 4 |
| | Mean (SD) (ng/mL) | 10.6 (17.5) | 1440 (2550) | 559 (714) |
| Day 64 | Number Analyzed | 6 | 4 | 4 |
| | Mean (SD) (ng/mL) | 16.4 (17.4) | 568 (841) | 3100 (3900) |

^{a}Pharmacokinetics analysis set consisted of all participants who received study drug and had at least 1 measurable serum concentration.
^{b}Number analyzed is the number of participants available for analysis at the given time point.

TABLE 19
Number of Participants with Change from Baseline in (Cluster of Differentiation
38) CD38 Expression Level and Receptor Occupancy (PK analysis set).

| | | | |
|---|---|---|---|
| Pooled | TAK-079 | TAK-079 | TAK-079 |
| Placebo | 45 mg | 90 mg | 135 mg |

Overall Number of Participants

| | | | | |
|---|---|---|---|---|
| Analyzed^{a} | 5 | 6 | 6 | 5 |

Receptor Occupancy Plasma cells

| | | | | | |
|---|---|---|---|---|---|
| Baseline | Number Analyzed | 5 | 6 | 6 | 5 |
| | Count^{b} | 4 (80.0%) | 6 (100.0%) | 6 (100.0%) | 5 (100.0%) |
| EOT^{c} | Number Analyzed | 5 | 6 | 6 | 5 |
| | Count | 4 (80.0%) | 3 (50.0%) | 4 (66.7%) | 4 (80.0%) |

Receptor Occupancy Plasmablasts

| | | | | | |
|---|---|---|---|---|---|
| Baseline | Number Analyzed | 5 | 6 | 6 | 5 |
| | Count | 4 (80.0%) | 6 (100.0%) | 6 (100.0%) | 5 (100.0%) |
| EOT | Number Analyzed | 5 | 6 | 6 | 5 |
| | Count | 4 (80.0%) | 5 (83.3%) | 4 (66.7%) | 4 (80.0%) |

CD38+ NK Cells

| | | | | | |
|---|---|---|---|---|---|
| Baseline | Number Analyzed | 5 | 6 | 6 | 5 |
| | Count | 4 (80.0%) | 6 (100.0%) | 6 (100.0%) | 5 (100.0%) |
| EOT | Number Analyzed | 5 | 6 | 6 | 5 |
| | Count | 4 (80.0%) | 5 (83.3%) | 4 (66.7%) | 4 (80.0%) |

CD38+ B Cells

| | | | | | |
|---|---|---|---|---|---|
| Baseline | Number Analyzed | 5 | 6 | 6 | 5 |
| | Count | 4 (80.0%) | 6 (100.0%) | 6 (100.0%) | 5 (100.0%) |
| EOT | Number Analyzed | 5 | 6 | 6 | 5 |
| | Count | 4 (80.0%) | 5 (83.3%) | 4 (66.7%) | 4 (80.0%) |

CD38+ T Cells

| | | | | | |
|---|---|---|---|---|---|
| Baseline | Number Analyzed | 5 | 6 | 6 | 5 |
| | Count | 4 (80.0%) | 6 (100.0%) | 6 (100.0%) | 5 (100.0%) |
| EOT | Number Analyzed | 5 | 6 | 6 | 5 |
| | Count | 4 (80.0%) | 5 (83.3%) | 4 (66.7%) | 4 (80.0%) |

CD38+ Monocytes

| | | | | | |
|---|---|---|---|---|---|
| Baseline | Number Analyzed | 5 | 6 | 6 | 5 |
| | | Pooled | TAK-079 | TAK-079 | TAK-079 |
| | | Placebo | 45 mg | 90 mg | 135 mg |
| EOT | Count | 4 (80.0%) | 6 (100.0%) | 6 (100.0%) | 5 (100.0%) |
| | Number Analyzed | 5 | 6 | 6 | 5 |
| | Count | 4 (80.0%) | 5 (83.3%) | 4 (66.7%) | 4 (80.0%) |

^{a}Pharmacokinetics analysis set consisted of all participants who received study drug and had at least 1 measurable serum concentration.
^{b}Reported as count of participants with a change from baseline.
^{c}Baseline up to Day 85 (EOT).

TAK-079 peak exposure was greater than dose proportional over the dose range tested, 45 mg to 135 mg. After the first administration, a 3-fold increase in dose resulted in an approximately 100-fold increase in mean Cₘₐₓ from 57.5 ng/mL to 6130 ng/mL. This greater than dose proportional increase in exposure was generally maintained in the subsequent dosing intervals.

Individual values for time to reach Cₘₐₓ (tₘₐₓ) ranged between 33 and 168 hours after injection in each dosing interval for subjects with available and measurable concentration data. The majority of subjects reached maximum drug concentrations at 108 hours after the first and second dose of TAK-079 across all dose groups. No PK collection was specified between 5 hours and 168 hours after the third and fourth dose, and apparent tₘₐₓ was at 168 hours in these dosing intervals in all but 2 subjects.

### Biomarker Validation

Prevalidation characterization and technical validation were completed for flow cytometric assays to evaluate CD45+ lymphocytes, T cells, B cells, NK cells, monocytes, granulocytes, PBs, and plasma cells in whole blood. Additionally, this assay was validated to quantitatively determine the CD38 receptor occupancy and receptor density across the respective cell types. The number of participants with a change from the baseline in immune cells (i.e., plasma cells, plasmablasts, NK cells, B cells, T cells, monocytes, and lymphocytes) are presented in Table 20.

TABLE 20
Number of Participants with Change from Baseline in Immune Cell Subsets
from Baseline up to Day 85 End of Treatment (EOT) (PD analysis set).

| | Pooled | TAK-079 | TAK-079 | TAK-079 |
|---|---|---|---|---|
| | Placebo | 45 mg | 90 mg | 135 mg |
| Overall Number of Participants Analyzed^{a} | 5 | 6 | 6 | 5 |

Plasma cells/μL

| | | | | | |
|---|---|---|---|---|---|
| Baseline | Number Analyzed | 5 | 6 | 6 | 5 |
| | Count^{b} | 4 (80.0%) | 6 (100.0%) | 6 (100.0%) | 5 (100.0%) |
| EOT^{c} | Number Analyzed | 5 | 6 | 6 | 5 |
| | Count | 4 (80.0%) | 6 (100.0%) | 4 (66.7%) | 4 (80.0%) |

Plasmablasts/μL

| | | | | | |
|---|---|---|---|---|---|
| Baseline | Number Analyzed | 5 | 6 | 6 | 5 |
| | Count | 4 (80.0%) | 6 (100.0%) | 6 (100.0%) | 5 (100.0%) |
| EOT | Number Analyzed | 5 | 6 | 6 | 5 |
| | Count | 4 (80.0%) | 6 (100.0%) | 4 (66.7%) | 4 (80.0%) |

NK cells/μL

| | | | | | |
|---|---|---|---|---|---|
| Baseline | Number Analyzed | 5 | 6 | 6 | 5 |
| | Count | 4 (80.0%) | 6 (100.0%) | 6 (100.0%) | 5 (100.0%) |
| EOT | Number Analyzed | 5 | 6 | 6 | 5 |
| | Count | 4 (80.0%) | 6 (100.0%) | 4 (66.7%) | 4 (80.0%) |

B cells/μL

| | | | | | |
|---|---|---|---|---|---|
| Baseline | Number Analyzed | 5 | 6 | 6 | 5 |
| | Count | 4 (80.0%) | 6 (100.0%) | 6 (100.0%) | 5 (100.0%) |
| EOT | Number Analyzed | 5 | 6 | 6 | 5 |
| | Count | 4 (80.0%) | 6 (100.0%) | 4 (66.7%) | 4 (80.0%) |

T cells/μL

| | | | | | |
|---|---|---|---|---|---|
| Baseline | Number Analyzed | 5 | 6 | 6 | 5 |
| | Count | 4 (80.0%) | 6 (100.0%) | 6 (100.0%) | 5 (100.0%) |
| EOT | Number Analyzed | 5 | 6 | 6 | 5 |
| | Count | 4 (80.0%) | 6 (100.0%) | 4 (66.7%) | 4 (80.0%) |

Monocytes/μL

| | | | | | |
|---|---|---|---|---|---|
| Baseline | Number Analyzed | 5 | 6 | 6 | 5 |
| | Count | 4 (80.0%) | 6 (100.0%) | 6 (100.0%) | 5 (100.0%) |
| EOT | Number Analyzed | 5 | 6 | 6 | 5 |
| | Count | 4 (80.0%) | 6 (100.0%) | 4 (66.7%) | 4 (80.0%) |

Lymphocytes/μL

| | | | | | |
|---|---|---|---|---|---|
| Baseline | Number Analyzed | 5 | 6 | 6 | 5 |
| | Count | 4 (80.0%) | 6 (100.0%) | 6 (100.0%) | 5 (100.0%) |
| EOT | Number Analyzed | 5 | 6 | 6 | 5 |
| | Count | 4 (80.0%) | 6 (100.0%) | 4 (66.7%) | 4 (80.0%) |

^{a}PD analysis set included all participants who received study drug and had at least 1 postdose PD measurement.
^{b}Reported as count of participants with a change from baseline.
^{c}Baseline up to Day 85 (EOT).

### Target Engagement Based on Receptor Occupancy Flow Cytometric Analyses

The CD38 receptor occupancy assay was developed to evaluate changes in CD45+ lymphocytes, T cells, B cells, NK cells, monocytes, granulocytes, PBs and plasma cells in whole blood. Additionally, CD38 expression and TAK-079 receptor occupancy was evaluated on all the respective cell types by comparing CD38 fluorescence signal for 2 independent flow cytometry samples containing either labeled TAK-079 (for quantification of “free” CD38 receptor) or labeled TSF-19 (non-competitive CD38 antibody for quantification of “total” CD38 receptor).

Trends in PD profiles were most evident after the first dose. Data in subsequent dosing intervals were confounded by dose holds and premature discontinuation (Section of “Extent of Exposure”), as well as other reasons related to sample availability and condition.

The NK cell population is the most abundant CD38-expressing cell population in peripheral blood, and receptor occupancy on this cell type can be used as a surrogate marker for CD38 engagement on target cells. TAK-079 engaged the CD38 target on CD38+ NK cells in a dose-dependent manner, with median receptor occupancy increasing from 43.8% to 88.4% in the studied dose range of 45 mg to 135 mg one day after the first dose. The trend for dose-dependent target engagement was less apparent with subsequent doses, such that a 90 mg dose reached similar or greater mean receptor occupancy compared to a 135 mg dose. Maximum receptor occupancy was achieved at approximately 4 to 7 days after drug administration for all dose groups and returned to near baseline before the next dosing interval, with exception of the 90 mg dose group in which receptor occupancy did not fully return to baseline starting from the second dosing interval.

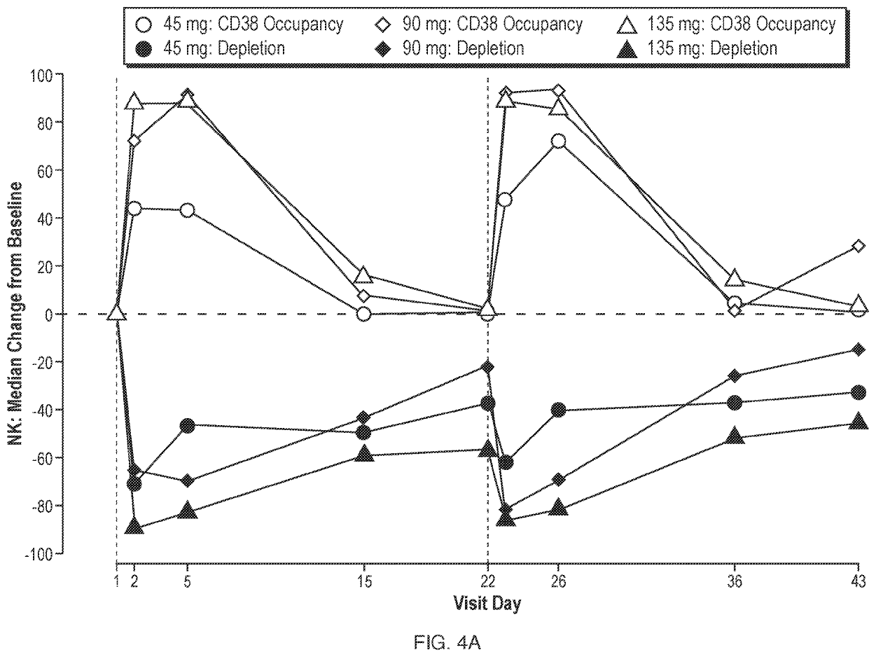

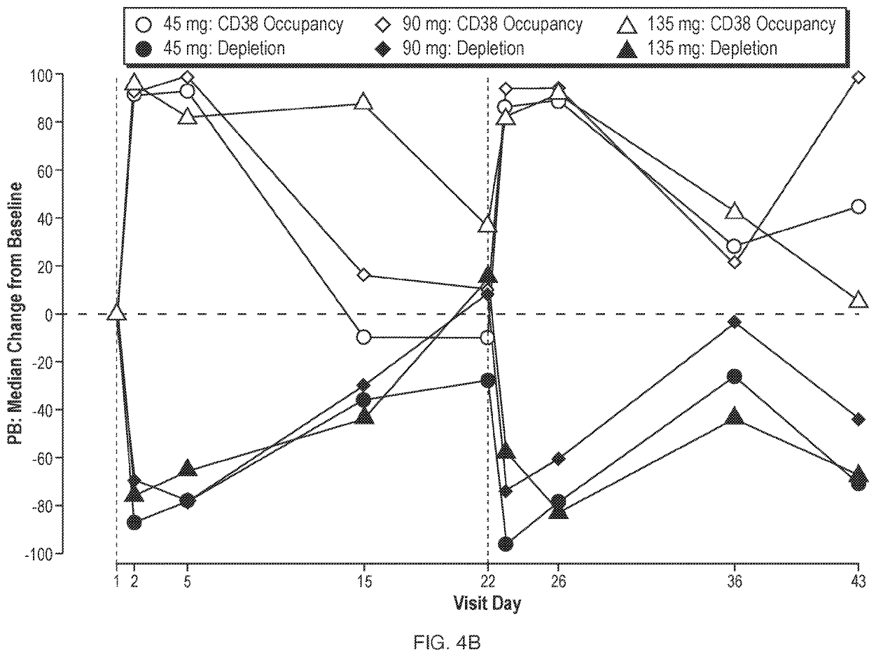

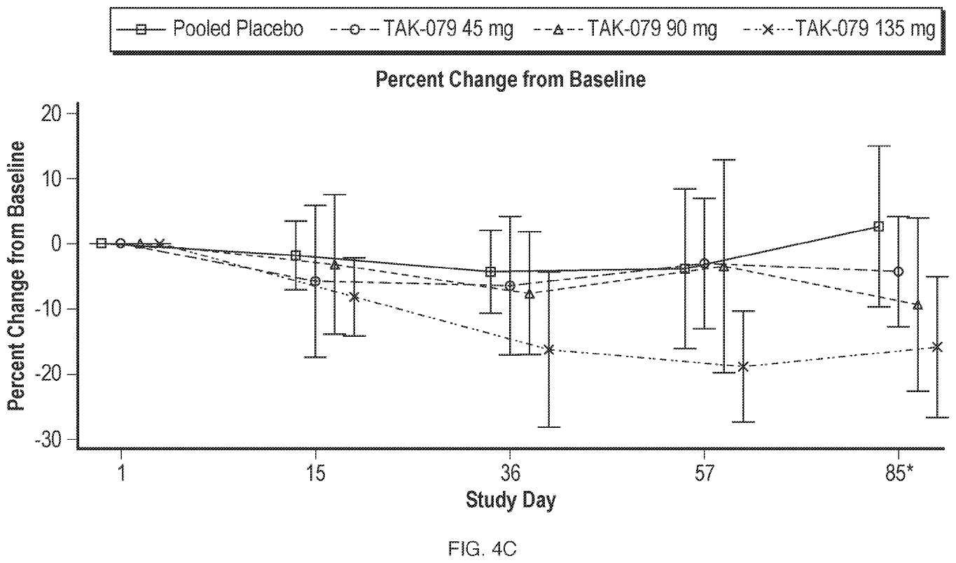

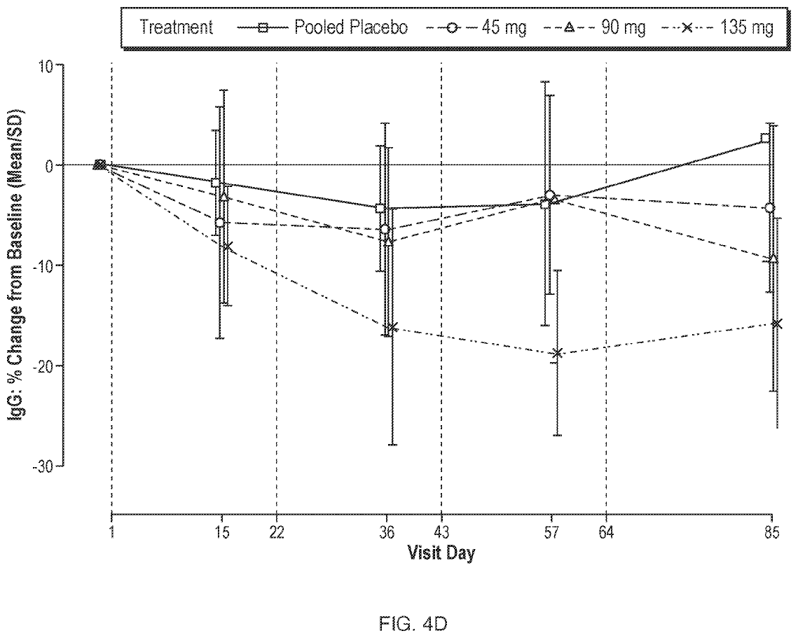

Target engagement was accompanied by changes in absolute cell counts of CD38+ NK cells. Corresponding reductions in CD38+ NK cells were approximately similar for all TAK-079 dose groups, with −71.5%, −65.5%, and −90.0% median change from baseline observed for 45 mg, 90 mg, and 135 mg, respectively (FIG. 4A). These reductions occurred at 2 days after the first dose and did not show complete return to baseline by the next dose administration. Similar trends were observed during subsequent dosing intervals. CD38+ NK cells were depleted by 33.7% change from baseline at the end of treatment (Day 85) across the pooled TAK-079 group. Placebo-treated subjects showed no reductions from baseline in CD38+ NK cells throughout the study.

Changes in absolute counts of PBs were examined because these cells represent the target cells for TAK-079 that can be quantified in peripheral blood. Reductions in absolute PB counts were similar across TAK-079 dose groups. The maximum effects were observed one day after the first dose with −87.0%, −69.4%, and −75.8% median change from baseline for 45 mg, 90 mg, and 135 mg, respectively (FIG. 4B). PB counts returned to baseline before the second dose administration. Similar trends were observed during subsequent dosing intervals, albeit PB depletion was less prominent.

### Downstream Pharmacology of Targeting CD38

Potential effects on plasma cells were assessed indirectly because these cells predominantly reside in tissues (such as bone marrow), with little presence in peripheral blood. Therefore, concentrations of total IgA, IgG, and IgM in serum were evaluated as a surrogate biomarker for TAK-079 effects on plasma cells in tissues.

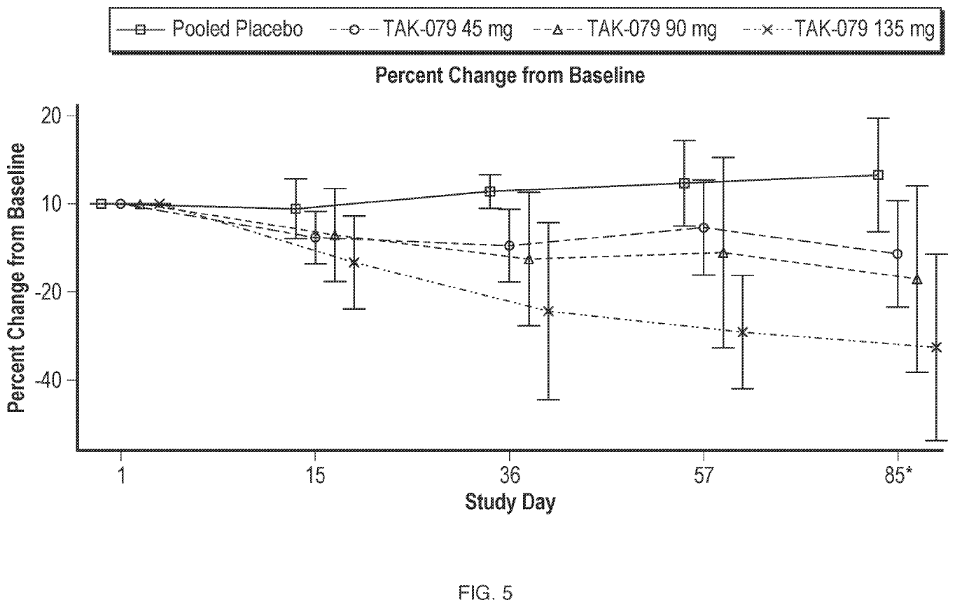

Multiple administrations of either 45 mg or 90 mg dose of TAK-079 resulted in modest reductions in IgG of less than 10% mean decrease from baseline at any given time point and did not show substantial difference compared to the placebo group, which had approximately 5% maximum mean decrease from baseline during the dosing period (FIG. 4C and FIG. 4D). A 135 mg dose of TAK-079 resulted in 18.8% maximum mean decrease from baseline in IgG. Depletion of IgA trended towards dose-dependency, with 11.3%, 17.1% and 32.6% maximum mean decrease from baseline in IgA achieved with 45 mg, 90 mg, and 135 mg, respectively (FIG. 5). Reductions in IgM concentrations were similar in all dose groups, with approximately 11.3% to 16.7% mean decrease from baseline across the pooled TAK-079 group during the dosing period. Reductions in immunoglobulins generally did not return to baseline levels by Day 85, which was the last time point examined in the study.

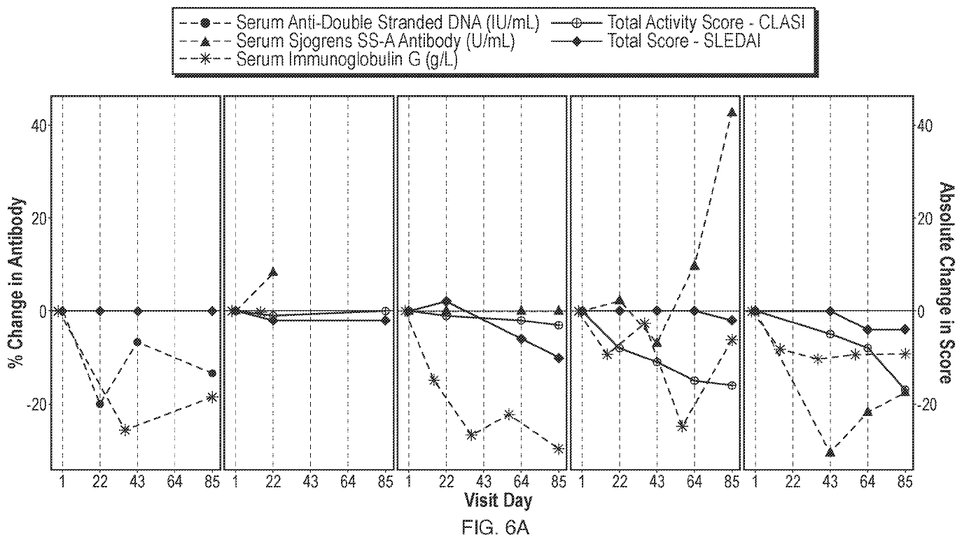

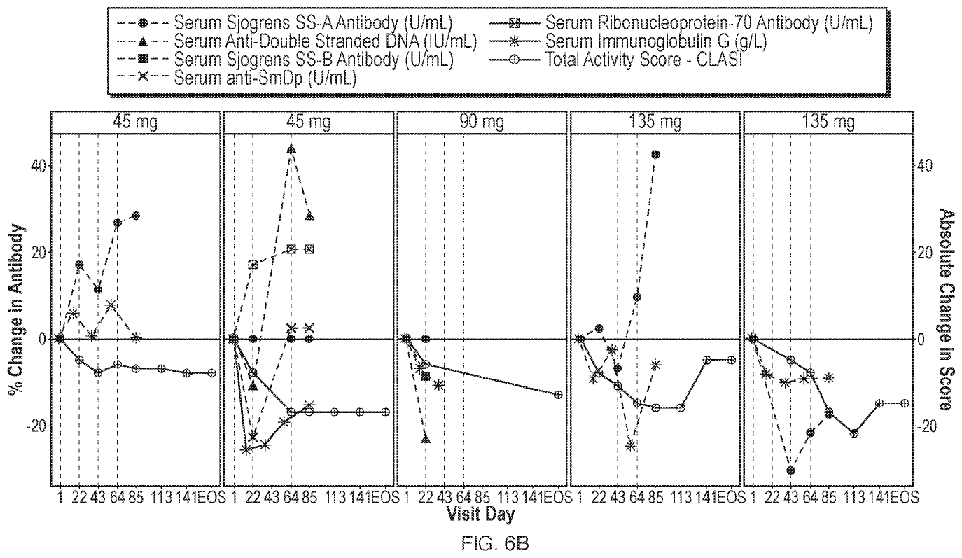

In addition to changes in total immunoglobulin concentrations, the effects of TAK-079 were assessed for changes in serum concentrations of autoantibodies in subjects positive for a given autoantibody at baseline. There were 6 main autoantibodies for which subjects were positive: anti-dsDNA, anti-SmD^{P}, beta-2 glycoprotein 1 IgM, ribonucleoprotein-70, Sjogrens SS-A and Sjogrens SS-B. Many subjects were positive for anti-dsDNA (9 of total 22 subjects) or Sjogrens SS-A (14 of total 22 subjects). Changes in autoantibody concentrations did not appear to be dose-dependent and generally did not show strong concordance with changes in total immunoglobulins (FIG. 6A and FIG. 6B). Maximum reductions in autoantibody concentrations were approximately 20% mean decrease from baseline for all evaluated autoantibodies.

### CyTOF

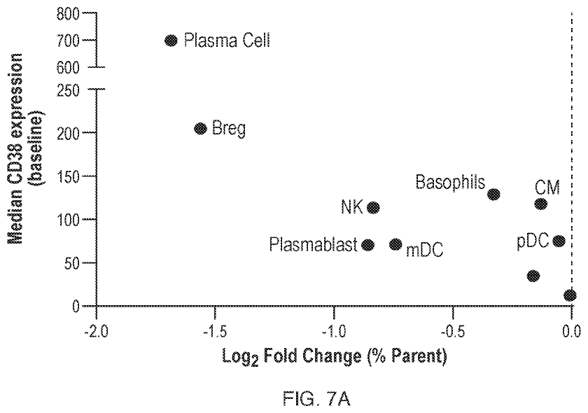

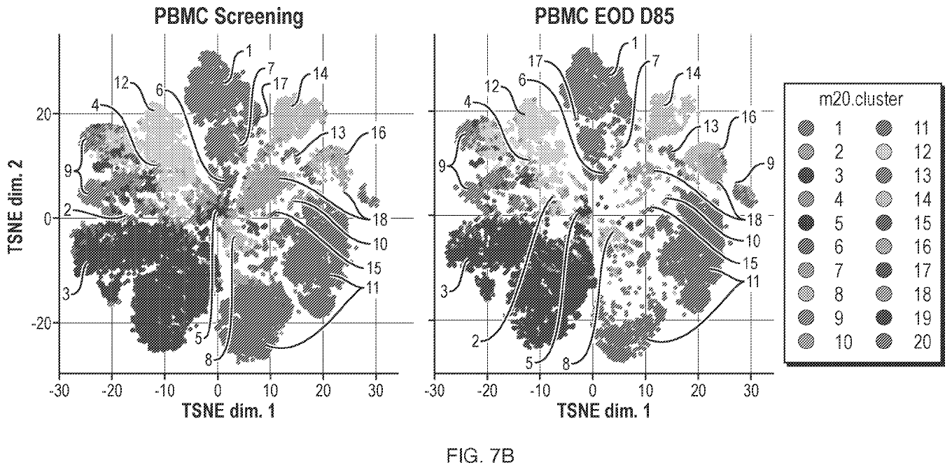

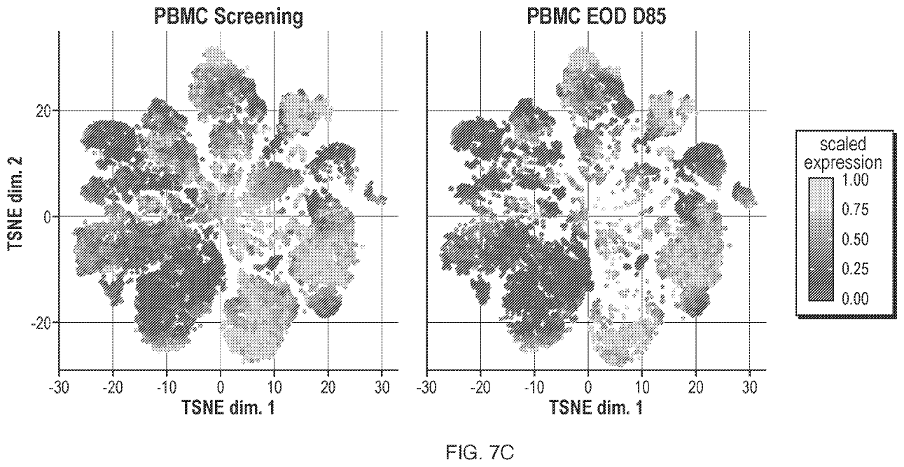

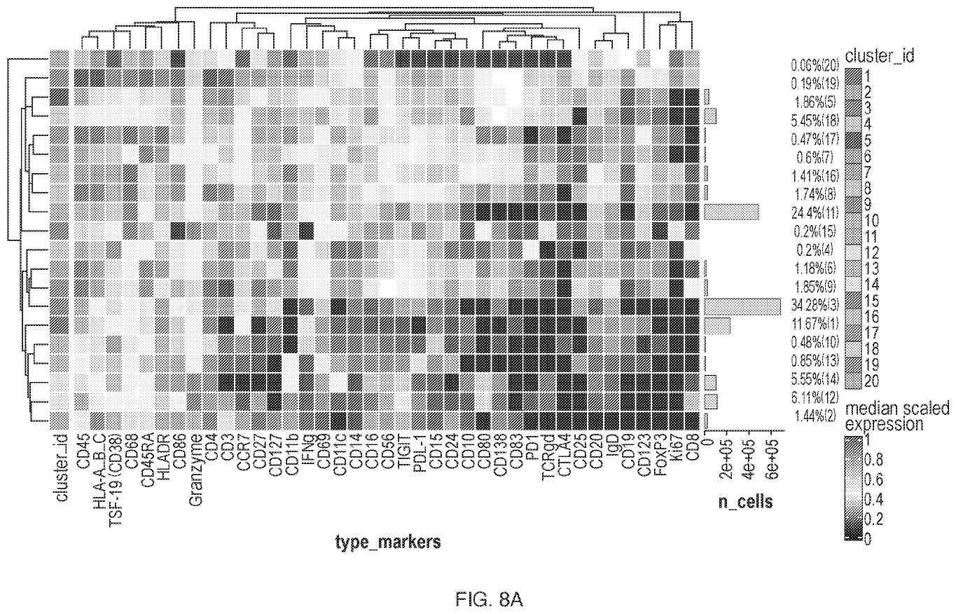

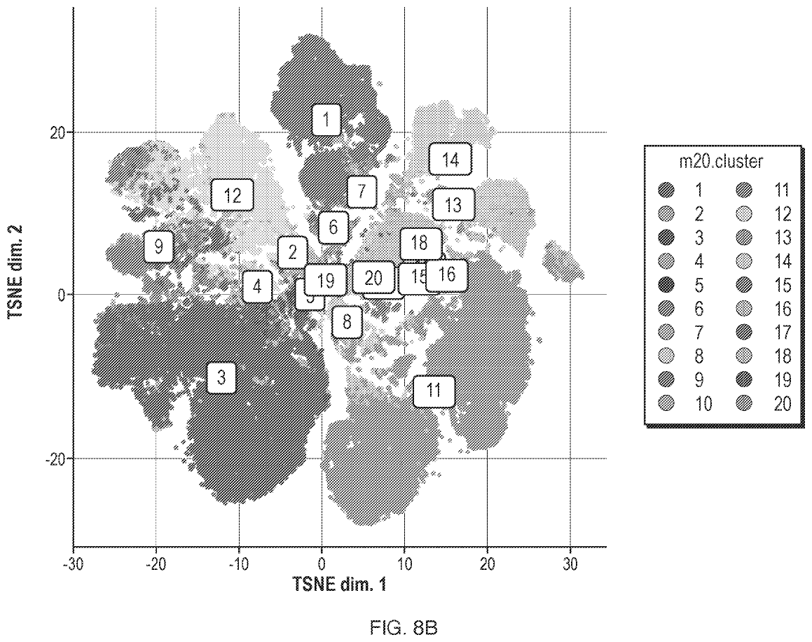

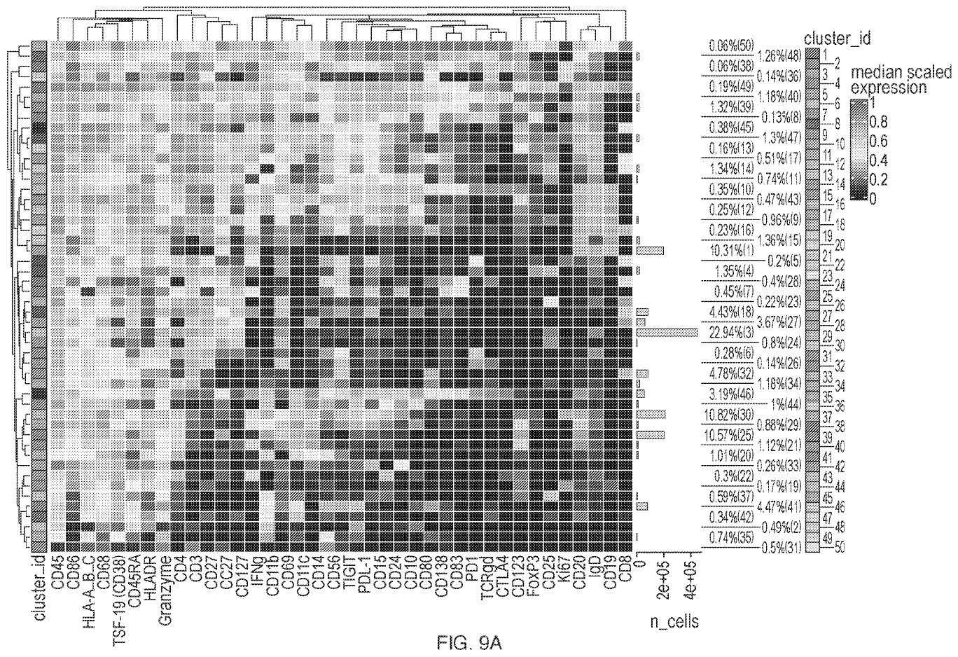

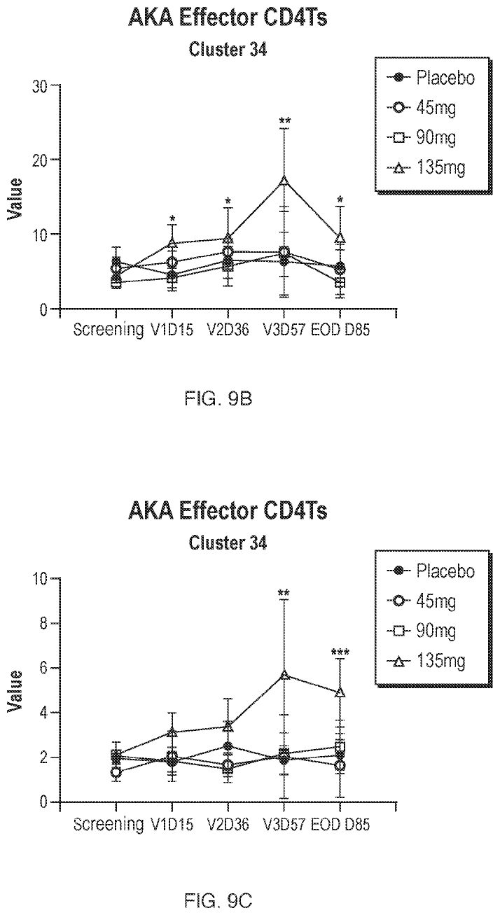

To gain further insight into the broad immune landscape changes induced by mezagitamab, CyTOF analysis was performed on PBMC samples collected during the study. These results showed a generalized trend that CD38 expression was correlated with the extent of cell depletion (FIG. 7A). Plasma cells, Bregs, NK cells and plasmablasts were among the most impacted populations, in alignment with the receptor occupancy data for a subset of these cells. Additionally, the unsorted CyTOF data was subjected to FlowSOM clustering and tSNE visualization (FIG. 7B, FIG. 7C, FIG. 8A, and FIG. 8B). These data showed changes in cluster size specifically around clusters expressing high levels of CD38. To obtain more information on specific clusters undergoing substantial alteration, cluster number was increased from 20 to 50 (FIG. 9A). This more granular analysis revealed two clusters of CD8+ and CD4+(32 and 34, FIGS. 9B and 9C) that express granzyme, are CCR7−, and are CD45RAlo/−. Assessing treatment effect on this population revealed an increase in the prevalence of these populations, which appeared to dependent on time on treatment and/or dose. Taken together, data showed that mezagitamab targets high CD38-expressing cells, resulting in their depletion and overall reduction of CD38 signal in immune cells.

### IFN Gene Signature

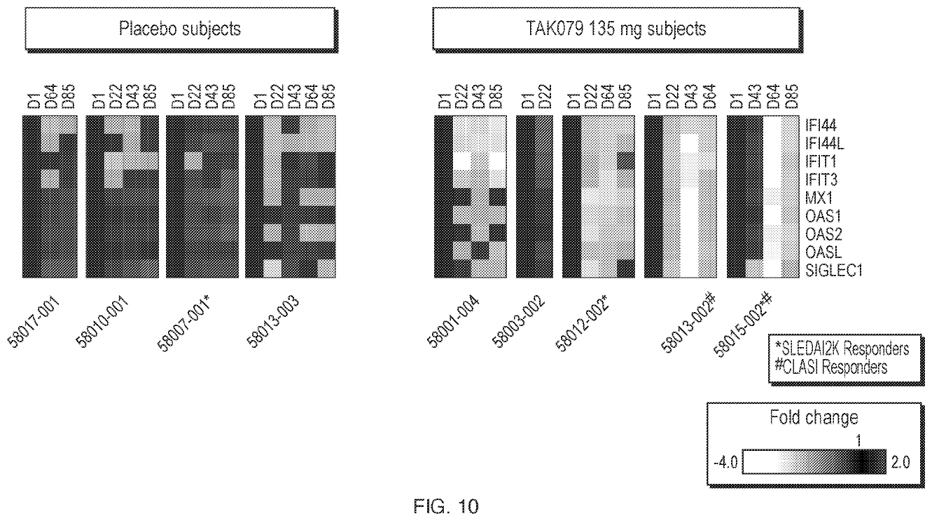

Whole blood RNA samples were collected at discrete timepoints throughout the mezagitamab treatment course and subjected to nanostring's autoimmune profiling panel. Genes associated with a Type 1 IFN response were identified to be downregulated in patients in the 135 mg cohort compared to placebo (FIG. 10). This response was most pronounced in the patients that saw a strong cutaneous response based on CLASI score.

### Immunogenicity Results

Changes made to immunogenicity analysis after the final SAP are described in Section of “Changes to Immunogenicity Analysis.”

A baseline and at least 1 postbaseline ADA assessment was performed for 21 subjects in the immunogenicity set. One subject had a missing assessment at baseline and so was not included in the immunogenicity set.

Subjects with a baseline positive ADA result were considered to have pre-existing ADAs. Three subjects in the placebo group, no subjects in the TAK-079 45 mg group, and 1 subject in each of the TAK-079 90 mg and 135 mg groups had pre-existing ADAs, giving an overall pre-existing ADA incidence rate of 23.8% (Table 21). The high proportion of subjects with pre-existing ADAs is likely to be the result of active autoimmune disease in these subjects with SLE (Faustini et al. (2021) Arthritis Res. Ther. 23(1): 211-23).

Subjects with a baseline positive ADA result and with ≥4-fold postbaseline increase in titer versus baseline titer were considered to have treatment-boosted ADA; however, no subjects met these criteria in this study. Subjects with a baseline negative ADA result and any postdose positive ADA result were considered to have treatment-emergent ADAs. No subjects in the placebo group, 2 subjects in each of the TAK-079 45 mg and 135 mg groups, and 1 subject in the TAK-079 90 mg group had treatment-emergent ADAs, giving an overall treatment-induced ADA incidence rate of 23.8% (Table 21). Among subjects with treatment-emergent ADA, 4 (19% of total subjects) were transiently ADA positive and only 1 (4.8% of total subjects) was persistently ADA positive.

The overall ADA prevalence, including both pre-existing and treatment-emergent ADA at any point in time was 47.6% (10 of 21 subjects) (Table 21). This proportion was similar across treatment groups, including the placebo group.

The minimum required dilution or the minimum titer in this study was 20 and titer was only assessed for ADA positive samples. Among all subjects with treatment-emergent ADAs, the medium titer was 200 and the range was 20 to 327,680 (Table 21). No correlation between ADA titer and the dose of drug administered was observed.

Immunogenicity was not associated with clinically significant AEs or hypersensitivity reactions, even in the subject with persistently positive ADA response. AEs and efficacy outcomes were closely assessed in 2 subjects (1 subject in the TAK-079 45 mg group and 1 subject in the TAK-079 135 mg group) who had ADA titers >40,000. Both subjects were negative for ADA at baseline but presented with dysregulated immune function at study start based on their medical history, which may partly explain the development of high ADA titers. No clinically relevant AEs or hypersensitivity reactions were observed in these subjects, and efficacy outcomes were consistent with study group response. Due to the limited number of subjects, the drug dose-dependence of immunogenicity could not be assessed, however, no obvious difference in the ADA positive response among the different treatment dosing groups was observed (Table 21).

Individual subject figures of serum TAK-079 concentrations over time alongside ADA results did not reveal any relationship between Cₘₐₓ and ADA response. At the individual level, Cₘₐₓ for subjects with treatment-emergent positive (transient or persistent) ADA response was generally lower following positive ADA assessment. However, comparable decreases in drug concentrations with continued dosing was also observed in subjects with negative ADA response. In addition, there was a substantial variability between subjects in Cₘₐₓ irrespective of ADA, for example, a geometric % CV of 186.3% was reported for the TAK-079 45 mg group at dose 1, in which all subjects had negative ADA status. Overall, data is too limited and variable to determine the impact of immunogenicity on the PK of TAK-079, but there was no apparent trend for differences in exposures for subjects with positive ADA response compared to those with negative ADA response.

TABLE 21
Summary of ADA Response (Immunogenicity Set)

| | Number (%) | | | | | | | |
|---|---|---|---|---|---|---|---|---|
| | Pooled | TAK-079 | TAK-079 | TAK-079 | Total | | | |
| | Placebo | 45 mg | 90 mg | 135 mg | TAK-079 | | Overall | |
| ADA Response | (N = 5) | (N = 6) | (N = 5) | (N = 5) | (N = 16) | | (N = 21) | |
| Negative ADA | 2 (40.0) | 4 (66.7) | 3 (60.0) | 2 (40.0) | 9 | (56.3) | 11 | (52.4) |
| Pre-existing ADA^{a} | 3 (60.0) | 0 | 1 (20.0) | 1 (20.0) | 2 | (12.5) | 5 | (23.8) |
| Treatment-emergent transient | 0 | 2 (33.3) | 1 (20.0) | 1 (20.0) | 4 | (25.0) | 4 | (19.0) |
| Treatment-emergent persistent | 0 | 0 | 0 | 1 (20.0) | 1 | (6.3) | 1 | (4.8) |
| ADA prevalence^{d} | 3 (60.0) | 2 (33.3) | 2 (40.0) | 3 (60.0) | 7 | (43.8) | 10 | (47.6) |
| ADA incidence^{e} | 0 | 2 (33.3) | 1 (20.0) | 2 (40.0) | 5 | (31.3) | 5 | (23.8) |

ADA: antidrug antibody.
Baseline value is defined as the last observed value before the first dose of study medication.
^{a}Pre-existing ADA response is defined as any subject with positive ADA status at baseline and either negative postbaseline samples or positive postbaseline samples with a liter not exceeding a 4-fold increase from baseline.
^{b}Treatment-emergent transient ADA response is defined as subjects who have a negative ADA status at baseline and confirmed positive ADA status in I or 2 post baseline assessment(s).
^{c}Treatment-emergent persistent ADA response is defined as subjects who have a negative ADA status at baseline and confirmed positive ADA status in >2 post baseline assessments.
^{d}ADA prevalence is the proportion of the study population having ADA, including pre-existing ADA, at any point in time
^{e}ADA incidence is the proportion of the study population with treatment-emergent ADA (including both transient and persistent ADA) or treatment-boosted ADA at any point in time. Treatment-boosted ADA is defined as >4-fold postbaseline increase in titer versus baseline titer; however, no subjects met the criteria for treatment-boosted ADA in this study.

TABLE 22
Summary of ADA Titer (Immunogenicity Set)

| | | | | | |
|---|---|---|---|---|---|
| | Pooled | TAK-079 | TAK-079 | TAK-079 | Total |
| Time Point | Placebo | 45 mg | 90 mg | 135 mg | TAK-079 |
| Statistic | (N = 5) | (N = 6) | (N = 5) | (N = 5) | (N = 16) |

Baseline

| | | | | | |
|---|---|---|---|---|---|
| n | 3 | 0 | 1 | 1 | 2 |
| Median | 320.0 | | 160.0 | 80.0 | 120.0 |
| Min, max | 20, 640 | | 160, 160 | 80, 80 | 80, 160 |

Dosing 2, D 22

| | | | | | |
|---|---|---|---|---|---|
| n | 1 | 0 | 1 | 1 | 2 |
| Median | 20.0 | | 160.0 | 20.0 | 90.0 |
| Min, max | 20, 20 | | 160, 160 | 20, 20 | 20, 160 |

Dosing 3, D 43

| | | | | | |
|---|---|---|---|---|---|
| n | 2 | 0 | 1 | 1 | 2 |
| Median | 330.0 | | 320.0 | 2560.0 | 1440.0 |
| Min, max | 20, 640 | | 320, 320 | 2560, 2560 | 320, 2560 |

Dosing 4, D 64

| | | | | | |
|---|---|---|---|---|---|
| n | 2 | 2 | 0 | 1 | 3 |
| Median | 30.0 | 20,500.0 | | 80.0 | 80.0 |
| Min, max | 20, 40 | 40, 40,960 | | 80, 80 | 40, 40,960 |

End of treatment, D 85

| | | | | | |
|---|---|---|---|---|---|
| n | 3 | 2 | 0 | 2 | 4 |
| Median | 320.0 | 163,860.0 | | 20,640.0 | 20,640.0 |
| Min, max | 20, 640 | 40, 327,680 | | 320, 40,960 | 40, 327,680 |

ADA: antidrug antibody; D: study day; max: maximum; min: minimum.
Baseline value is defined as the last observed value before the first dose of study medication.

### Analysis of Efficacy

The exploratory objectives assessed the effects of repeated administration of TAK-079 on disease activity using clinical rating scales.

### CLASI

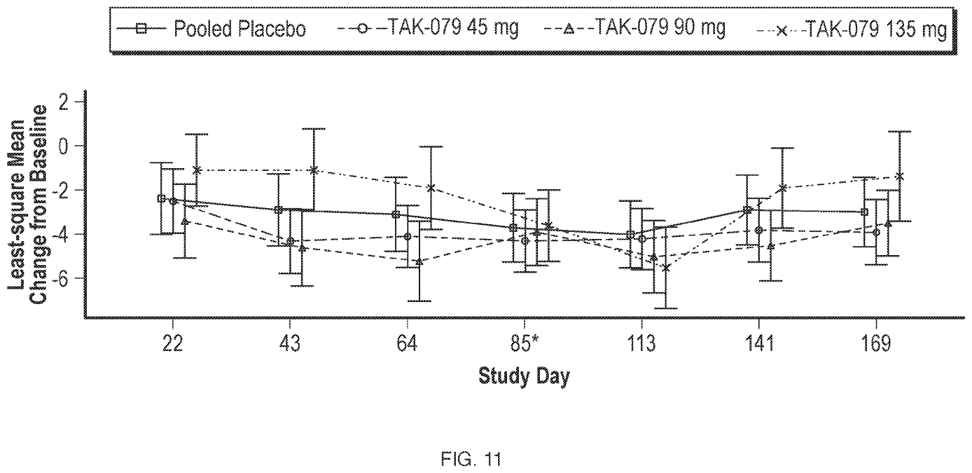

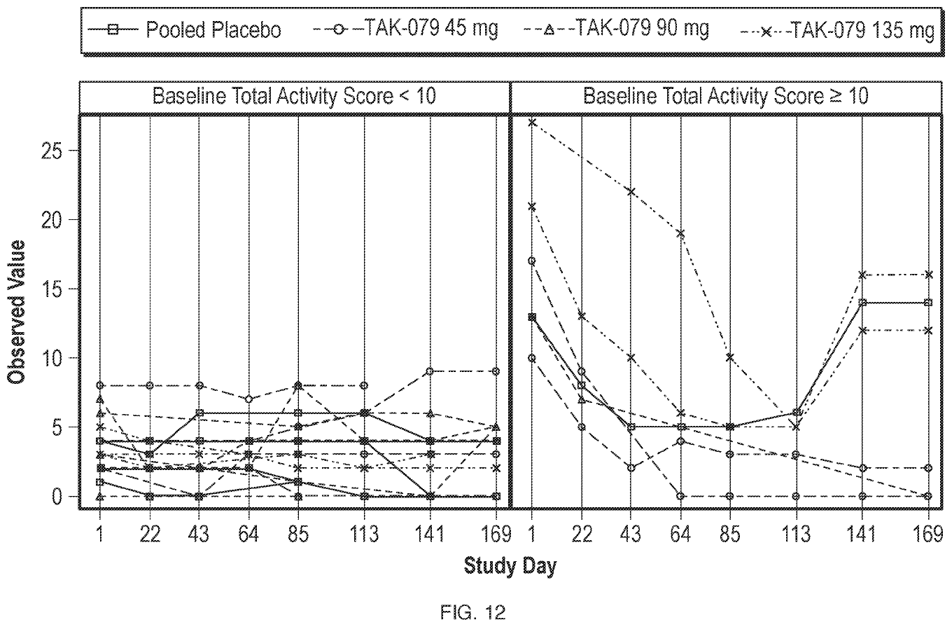

There was moderate improvement from baseline to end of treatment in the CLASI total activity score and no clear change in the CLASI total damage score, with no observable differences between the treatment groups. At baseline, the mean CLASI total activity scores were lower in the placebo (4.8), TAK-079 45 mg (7.2), and TAK-079 90 mg (5.2) groups compared with the TAK-079 135 mg (11.8) group (Table 23). At the end of treatment on Day 85, the change from baseline in total activity score (LS mean) was similar in the placebo group (−3.7) compared with the TAK-079 treatment groups (TAK-079 45 mg: −4.3; TAK-079 90 mg: −3.9; TAK-079 135 mg: −3.6) (Table 23 and FIG. 11). The number of responders, defined as subjects whose score decreased from baseline either by at least 4 points or by at least 20%, was also similar across treatment groups at this time point (Table 23). When individual total activity scores were assessed, all subjects with a baseline CLASI score of >10 met responder criteria at end of treatment (FIG. 12, Table 24). At the end of treatment on Day 85, there was little change from baseline in total damage score (LS mean) both in the placebo group (0.0) and the TAK-079 treatment groups (TAK-079 45 mg: −1.3; TAK-079 90 mg: 0.0; TAK-079 135 mg: 0.4).

TABLE 23
Summary and Analysis of CLASI Total Activity
Score by Visit (Safety Analysis Set)

| | | | | |
|---|---|---|---|---|
| | Pooled | TAK-079 | TAK-079 | TAK-079 |
| Time Point; Parameter | Placebo | 45 mg | 90 mg | 135 mg |
| Statistics | (N = 5) | (N = 6) | (N = 6) | (N = 5) |

Baseline; observed values

| | | | | |
|---|---|---|---|---|
| n | 5 | 6 | 6 | 5 |
| Mean (SD) | 4.8 (4.76) | 7.2 (5.81) | 5.2 (4.62) | 11.8 (11.37) |
| Median | 4.0 | 6.0 | 4.5 | 5.0 |
| Min, max | 1, 13 | 2, 17 | 0, 13 | 3, 27 |

End of treatment (Day 85); Change from baseline

| | | | | |
|---|---|---|---|---|
| n | 5 | 6 | 5 | 5 |
| Mean (SD) | −1.4 (3.85) | −4.0 (7.04) | 0.0 (3.54) | −7.2 (8.58) |
| Median | 0.0 | −1.0 | −1.0 | −3.0 |
| LS mean (SEM)^{a} | −3.7 (1.56) | −4.3 (1.39) | −3.9 (1.53) | −3.6 (1.61) |
| Difference in LS mean (SEM)^{a} | | −0.6 (2.09) | −0.2 (2.12) | 0.1 (2.31) |
| 95% CI of the difference | | −4.9, 3.7 | −4.5, 4.1 | −4.6, 4.8 |

End of treatment (Day 85); Subjects meeting clinical response criteria^{b}

| | | | | |
|---|---|---|---|---|
| n | 5 | 6 | 5 | 5 |
| Responders^{c} | 2 (40.0) | 3 (50.0) | 2 (40.0) | 3 (60.0) |

CLASI: Cutaneous Lupus Erythematosus Disease Area and Severity Index; max: maximum; min: minimum.
MMRM: mixed-effect model for repeated measures.
Baseline value is defined as the last observed value before the first dose of study medication.
^{a}From a MMRM analysis over all postbaseline visits, with the change from baseline as the outcome, treatment, visit, and (treatment × visit) interaction terms as the factors, and baseline value and (baseline × visit) interaction as the covariates. Kenward-Rogers method is used to calculate the degrees of freedom and covariance matrix is based on autoregressive (1) covariance structure. The pooled placebo group is used as the controlled group for the difference (active-placebo) in LS mean and its 95% CI.
^{b}Only patients who have a baseline score above zero are included.
^{c}Responder is defined as a subject whose score decreased from baseline either by at least 4 points or by at least 20%. 95% CI for responder percentage is based on the Wilson method.

TABLE 24
CLASI Responder Analysis

| Subject | Study | | Baseline | | EOT | EOT Clinical | |
|---|---|---|---|---|---|---|---|
| ID | Group | | Scores | Baseline Clinical Presentation | Scores | Presentation | Δ |
| 58004-003 | Placebo | | 13 | Verrucous hypertrophic dyspigmentation | 5 | Both resolved | −8 |
| | | | | on nose, red erythema on face | | | |
| 58004-002 | 45 | mg | 10 | Diagnosis of eczema since 2007 ongoing; | 3 | Red erythema resolved | −7 |
| | | | | red erythema of chest and face | | | |
| 58010-002 | 45 | mg | 17 | Red erythema + dyspigmentation of | 0 | Red erythema resolved; | −17 |
| | | | | abdomen, back, hands, scalp | | some dyspigmentation | |
| | | | | | | left | |
| 58007-002 | 90 | mg | 13 | Diagnosis of rash related to SLE since | 0 | All erythema resolved | −13 |
| | | | | 2016 ongoing; red, scaly erythema on | | after 2 doses | |
| | | | | ears, red erythema on nose, shoulder, | | | |
| | | | | V-neck area | | | |
| 58013-002 | 135 | mg | 21 | Red erythema + scaling + | 5 | Only dyspigmentation | −16 |
| | | | | dyspigmentation of arms, chest, ears, | | left; red erythema + | |
| | | | | hands, nose, neck, shoulders and feet | | scaling resolved | |
| | | | | | | (besides face, scalp) | |
| 58015-002 | 135 | mg | 27 | Dark red erythema & purple, violaceus | 10 | Arms only present with | −17 |
| | | | | crusted hemorrhagic erythema on arms; | | red erythema; most | |
| | | | | red erythema + scaling, dyspigmentation | | erythema have pink | |
| | | | | & scarring on back, chest, ears, hands, | | instead of red, scaling | |
| | | | | legs, neck, shoulders, scalp; scarring | | has decreased | |
| | | | | of nose and face | | | |

EOT is Day 85 visit. For patient 58007-002: Day 85 data were not available, the indicated value is based on the follow-up visit.

### SLEDAI-2K

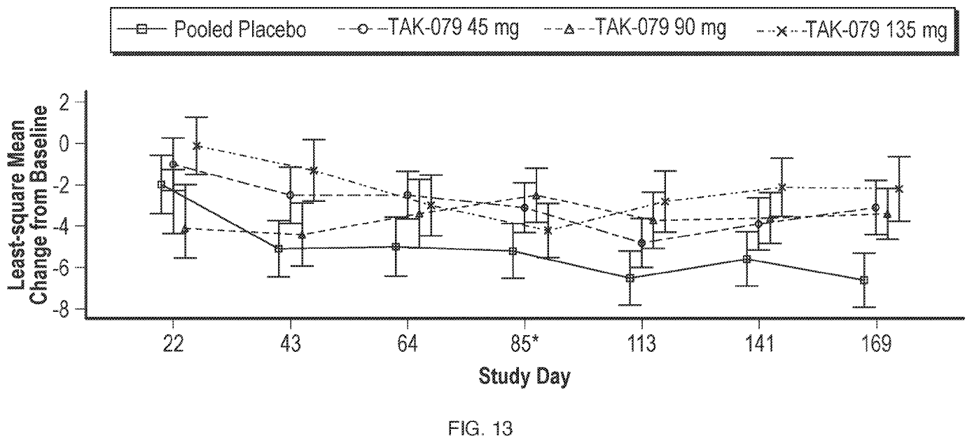

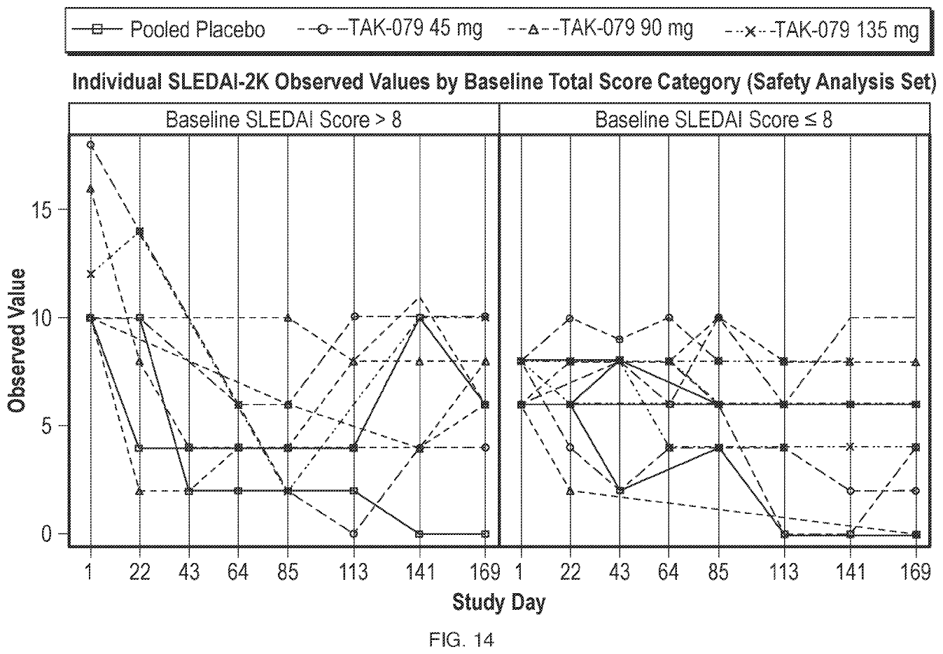

Mild to moderate improvement from baseline to end of treatment in SLEDAI-2K total scores was observed in this study. There were no observable differences across the treatment groups. At baseline, the mean SLEDAI-2K total scores were similar in the placebo (8.4), TAK-079 45 mg (9.7), TAK-079 90 mg (9.7), and TAK-079 135 mg (8.8) groups (Table 25). At the end of treatment on Day 85, the change from baseline in total score (LS mean) was similar in the placebo group (−5.2) compared with the TAK-079 treatment groups (TAK-079 45 mg: −3.1; TAK-079 90 mg: −2.5; TAK-079 135 mg: −4.2) (Table 25 and FIG. 13). The number of responders, defined as subjects whose score decreased from baseline by at least 4 points, was similar across treatment groups at this time point (Table 25). No trends were observed in individual SLEDAI-2K total scores (FIG. 14).

TABLE 25
Summary and Analysis of SLEDAI-2K Total Score by Visit (Safety Analysis Set)

| | | | | |
|---|---|---|---|---|
| | Pooled | TAK-079 | TAK-079 | TAK-079 |
| Time Point; Parameter | Placebo | 45 mg | 90 mg | 135 mg |
| Statistics | (N = 5) | (N = 6) | (N = 6) | (N = 5) |

Baseline; observed values

| | | | | |
|---|---|---|---|---|
| n | 5 | 6 | 6 | 5 |
| Mean (SD) | 8.4 (1.67) | 9.7 (4.27) | 9.7 (3.67) | 8.8 (1.79) |
| Median | 8.0 | 8.0 | 10.0 | 8.0 |
| Min, max | 6, 10 | 6, 18 | 6, 16 | 8, 12 |

End of treatment (Day 85); Change from baseline

| | | | | |
|---|---|---|---|---|
| n | 5 | 6 | 5 | 5 |
| Mean (SD) | −4.0 (3.16) | −3.7 (5.72) | −3.6 (6.07) | −3.6 (3.85) |
| Median | −4.0 | −3.0 | −4.0 | −2.0 |
| LS mean (SEM)^{a} | −5.2 (1.30) | −3.1 (1.17) | −2.5 (1.29) | −4.2 (1.29) |
| Difference in LS mean (SEM)^{a} | | 2.0 (1.76) | 2.7 (1.85) | 0.9 (1.82) |
| 95% CI of the difference | | (−1.5, 5.6) | (−1.0, 6.4) | (−2.7, 4.6) |

End of treatment (Day 85); Subjects meeting clinical response criteria^{b}

| | | | | |
|---|---|---|---|---|
| n | 5 | 6 | 5 | 5 |
| Responders^{b} | 3 (60.0) | 3 (50.0) | 3 (60.0) | 2 (40.0) |

Max: maximum; min: minimum; MMRM: mixed-effect model for repeated measures; SLEDAI-2K: Systemic Lupus Erythematosus Disease Activity Index - 2000.
Baseline value is defined as the last observed value before the first dose of study medication.
^{a}From a MMRM analysis over all postbaseline visits, with the change from baseline as the outcome, treatment, visit, and (treatment × visit) interaction terms as the factors, and baseline value and (baseline × visit) interaction as the covariates. Kenward-Rogers method is used to calculate the degrees of freedom and covariance matrix is based on autoregressive (1) covariance structure. The pooled placebo group is used as the controlled group for the difference (active-placebo) in LS mean and its 95% CI.
^{b}Responder is defined as a subject whose score decreased from baseline either by at least 4 points.

### PGA of Disease (VAS)

A mild to moderate improvement from baseline to end of treatment in the PGA of disease scores was detected. There were no observable differences between the treatment groups.

At baseline, the mean PGA of disease scores presented as disease severity on a 3 point scale were similar in the placebo (1.48), TAK-079 45 mg (1.70), TAK-079 90 mg (2.02), and TAK-079 135 mg (1.84) groups (Table 25).

At the end of treatment on Day 85, the change from baseline score (LS mean) was similar in the placebo group (−0.58) compared with the TAK-079 treatment groups (TAK-079 45 mg: −0.71; TAK-079 90 mg: −0.48; TAK-079 135 mg: −0.90) (Table 26). The number of responders, defined as a subject whose score decreased from baseline by at least 0.3 points, was similar across treatment groups at this time point (Table 26).

No trends in individual PGA of disease scores were observed.

When PGA of disease was presented as disease severity on a VAS scale, similar results were observed.

TABLE 26
Summary and Analysis of PGA of Disease (Disease Severity
on a 3 Point Scale) by Visit (Safety Analysis Set)

| | | | | |
|---|---|---|---|---|
| | Pooled | TAK-079 | TAK-079 | TAK-079 |
| Time Point; Parameter | Placebo | 45 mg | 90 mg | 135 mg |
| Statistics | (N = 5) | (N = 6) | (N = 6) | (N = 5) |

Baseline; observed values

| | | | | |
|---|---|---|---|---|
| n | 5 | 6 | 6 | 5 |
| Mean (SD) | 1.48 (0.277) | 1.70 (0.443) | 2.02 (0.371) | 1.84 (0.439) |
| Median | 1.60 | 1.75 | 2.10 | 1.70 |
| Min, max | 1.0, 1.7 | 1.1, 2.2 | 1.4, 2.4 | 1.4, 2.4 |

End of treatment (Day 85); Change from baseline

| | | | | |
|---|---|---|---|---|
| n | 5 | 6 | 5 | 5 |
| Mean (SD) | −0.50 (0.141) | −0.70 (0.477) | −0.52 (0.356) | −0.94 (0.666) |
| Median | −0.40 | −0.65 | −0.50 | −0.70 |
| LS mean (SEM) ^{a} | −0.58 (0.229) | −0.71 (0.199) | −0.48 (0.235) | −0.90 (0.221) |
| Difference in LS mean (SEM) ^{a} | | −0.12 (0.302) | 0.10 (0.346) | −0.32 (0.325) |
| 95% CI of the difference | | (−0.73, 0.48) | (−0.60, 0.79) | (−0.97, 0.34) |

End of treatment (Day 85); Subjects meeting clinical response criteria

| | | | | |
|---|---|---|---|---|
| n | 5 | 6 | 5 | 5 |
| Responders ^{b} | 5 (100.0) | 5 (83.3) | 4 (80.0) | 4 (80.0) |

Max: maximum; min: minimum; MMRM: mixed-effect model for repeated measures; PGA: Physician's Global Assessment; SLEDAI-2K: Systemic Lupus Erythematosus Disease Activity Index - 2000.
Baseline value is defined as the last observed value before the first dose of study medication.
^{a}From a MMRM analysis over all postbaseline visits, with the change from baseline as the outcome, treatment, visit, and (treatment × visit) interaction terms as the factors, and baseline value and (baseline × visit) interaction as the covariates. Kenward-Rogers method is used to calculate the degrees of freedom and covariance matrix is based on autoregressive (1) covariance structure. The pooled placebo group is used as the controlled group for the difference (active-placebo) in LS mean and its 95% CI.
^{b}Responder is defined as a subject whose score decreased from baseline by at least 0.3 points

### 44-Joint Assessment

Overall, there was moderate improvement from baseline to end of treatment in the total tender joint count, the total swollen joint count, and the total active joint count, with no observable differences between the treatment groups.

At baseline, the mean total tender joint count was similar across treatment groups. At the end of treatment on Day 85, the change from baseline (LS mean) was similar in the placebo group (−9.5) compared with the TAK-079 treatment groups (TAK-079 45 mg: −7.9; TAK-079 90 mg: −14.2; TAK-079 135 mg: −10.1).

At baseline, the mean total swollen joint count was similar across treatment groups. At the end of treatment on Day 85, the change from baseline (LS mean) was similar in the placebo group (−5.8) compared with the TAK-079 treatment groups (TAK-079 45 mg: −3.1; TAK-079 90 mg: −2.1; TAK-079 135 mg: −7.1).

At baseline, the mean total active joint count was similar across treatment groups. At the end of treatment on Day 85, the change from baseline (LS mean) was similar in the placebo group (−6.1) compared with the TAK-079 treatment groups (TAK-079 45 mg: −4.0; TAK-079 90 mg: −6.4; TAK-079 135 mg: −6.7).

### Statistical and Analytical Issues

### Handling of Dropouts or Missing Data

Efficacy data was analyzed using observed case data only based on a “missing at random” assumption.

### Interim Analyses and Data Monitoring

No interim analyses were performed for this study.

Cohort A and B treatment assignments were unblinded after all subjects in Cohorts A and B had completed the study and all data had been source data verified. The sponsor received treatment assignments only for individual subjects within the pertinent cohorts while remaining blinded to treatment assignments of subjects in the other cohorts. Details on the unblinding process and steps to minimize bias are described in Section of “Blinding”.

### Multicenter Studies

This was a multicenter study. No adjustments for study centers were made.

### Multiple Comparisons/Multiplicity

No multiplicity adjustments were made.

### Use of an Efficacy Subset of Subjects

An efficacy subset of subjects was not used in this study.

### Active-Control Studies Intended to Show Equivalence

This was not an active-control study intended to show equivalence.

### Examination of Subgroups

No subgroup analyses were performed in this study.

### PK, PD, Immunogenicity, and Efficacy Conclusions

Consistent with the literature and other SLE studies, most study subjects were female. There was a well-balanced racial representation in the study, with nearly equal percentages of African American and White study subjects.

TAK-079 exposure increase was greater than dose proportional over the dose range tested (45 to 135 mg). After the first administration, a 3-fold increase in dose resulted in an approximately 100-fold increase in mean Cₘₐₓ. This greater than dose proportional increase in exposure was generally maintained in the subsequent dosing intervals.

The majority of subjects reached maximum drug concentrations at 108 hours after the first and second dose of TAK-079 across all dose groups.

TAK-079 engaged the CD38 target on CD38+ NK cells in a dose-dependent manner after the first dose. Maximum receptor occupancy was achieved at approximately 4 to 7 days after drug administration for all dose groups. CD38+ NK cells were depleted by 33.7% change from baseline at the end of treatment (Day 85) in the pooled TAK-079 group. Placebo-treated subjects showed no reductions from baseline in CD38+ NK cells throughout the study.

Reductions in absolute PB counts were similar across TAK-079 dose groups. The maximum effects were observed 2 days after the first dose and reached mean change of −74.0% in the pooled TAK-079 group. Decrease in IgG was most prominent in the TAK-079 135 mg group, with 18.8% maximum mean decrease from baseline. Depletion of IgA trended towards dose-dependency, with 11.3%, 17.1%, and 32.6% maximum mean decrease from baseline in IgA achieved with 45 mg, 90 mg, and 135 mg TAK-079 doses, respectively. Reductions in IgM concentrations were similar in all dose groups, with approximately 11.3% to 16.7% mean decrease from baseline in the pooled TAK-079 group during the dosing period.

Changes in autoantibody concentrations did not appear to be dose-dependent and generally did not show strong concordance with changes in total immunoglobulins. Maximum reductions in autoantibody concentrations were approximately 20% mean decrease from baseline for all evaluated autoantibodies.

A total of 5 subjects from across the placebo and TAK-079 treatment groups had pre-existing ADA, for an overall mean rate of 23.8%. A further 5 subjects from the TAK-079 treatment groups had treatment-emergent ADA, giving an incidence of 23.8%. Immunogenicity was not associated with clinically significant AEs or hypersensitivity reactions and the dose-dependence of immunogenicity could not be assessed due to the limited number of subjects.

Four clinical rating scales were used to assesses exploratory efficacy in this study: CLASI, SLEDAI-2K, PGA, and 44-joint assessment (total tender joint count, total swollen joint count, and total active joint count). No differences were observed across treatment groups for any of these clinical rating scales. When individual CLASI total activity scores were assessed, all patients with a baseline CLASI score of >10 met responder criteria at end of treatment, suggesting there is a trend for more profound response among patients with higher CLASI score at baseline.

Overall, responder analyses for CLASI, SLEDAI-2K, and PGA did not reveal any observable differences across treatment groups.

### Example 4: Safety and Safety Biomarker Evaluation Extent of Exposure

A summary of study drug exposure and compliance is provided in Table 17. In total, 17 subjects were exposed to TAK-079. Mean compliance was 80.0% in the placebo group and 70.6% in the pooled (total) TAK-079 group.

Many subjects did not receive all 4 doses of study drug due to a number of factors, including the COVID-19 pandemic (see Section of “Impact of the COVID-19 Pandemic on Study Subjects”) and dose holds (see Section of “Dose Holds”). A minority of subjects in each treatment group received all 4 doses of study drug: 2 of 5 subjects in the placebo group, 2 of 6 subjects in the TAK-079 45 and 90 mg groups, and 1 of 5 subjects in the TAK-079 135 mg group.

### Dose Holds

Before receiving each study drug dose, subjects were evaluated for dosing criteria based on laboratory evaluations and events of clinical interest as defined in Table 11.

Overall, the number of dose holds was balanced across treatment groups: 3 subjects in the placebo group, 2 subjects in each of the TAK-079 45 and 135 mg groups, and 1 subject in the TAK-079 90 mg group had dose holds based on laboratory evaluations and 1 subject in the TAK-079 45 mg group had a dose hold based on an event of clinical interest.

### AEs

A pretreatment event (PTE) was defined as any untoward medical occurrence in a clinical investigation subject who had signed informed consent to participate in a study, but before administration of any study medication; it did not necessarily have to have a causal relationship with study participation.

An AE was defined as any untoward medical occurrence in a clinical investigation subject administered a drug; it did not necessarily have to have a causal relationship with this treatment. An AE can therefore be any unfavorable and unintended sign (example, a clinically significant abnormal laboratory finding), symptom, or disease temporally associated with the use of a drug, whether or not it is considered related to the drug. A TEAE is defined as an AE with an onset that occurs after receiving study drug. An SAE is an adverse event resulting in any of the following outcomes or deemed significant for any other reason: death; initial or prolonged inpatient hospitalization; life-threatening experience (immediate risk of dying); persistent or significant disability/incapacity; congenital anomaly; any event or symptom described in Table 27. TEAEs were defined as AEs that occurred after the first dose of study drug received in the treatment period and until the end of safety follow-up. The terms “serious TEAEs” and “treatment-emergent SAEs” can be considered interchangeable in this document. The severity of TEAEs will be graded using National cancer institute-Common Terminology Criteria for Adverse Events (NCI-CTCAE) version 4.0 definitions of Grade 1 through Grade 5. Grade 1 Mild; asymptomatic or mild symptoms; clinical or diagnostic observations only; intervention not indicated. Grade 2 Moderate; minimal, local or noninvasive intervention indicated; limiting age-appropriate instrumental activities of daily living. Grade 3 Severe or medically significant but not immediately life-threatening; hospitalization or prolongation of hospitalization indicated; disabling; limiting self care activities of daily living. Grade 4 Life-threatening consequences; urgent intervention indicated. Grade 5 Death related to AE.

TABLE 27
General List of Takeda Medically Significant AEs
Term

| | |
|---|---|
| Acute respiratory failure/acute respiratory | Hepatic necrosis |
| distress syndrome | |
| Torsade de pointes/ventricular fibrillation/ | Acute liver failure |
| ventricular tachycardia | |
| Malignant hypertension | Anaphylactic shock |
| Convulsive seizures | Acute renal failure |
| Agranulocytosis | Pulmonary hypertension |
| Aplastic anemia | Pulmonary fibrosis |
| Toxic epidermal necrolysis/Stevens-Johnson | Confirmed or suspected endotoxin shock |
| syndrome | |
| | Confirmed or suspected transmission of |
| | infectious agent by a medicinal product |
| | Neuroleptic malignant syndrome/malignant |
| | hyperthermia |
| | Spontaneous abortion/stillbirth and fetal death |

PTE and AE verbatim terms were coded by SOC and PT using MedDRA version 24.0.

An overview of TEAEs is presented in Table 28.

TABLE 28
Overview of TEAEs (Safety Analysis Set)

| | Pooled | TAK-079 | TAK-079 | | TAK-079 | Total | |
|---|---|---|---|---|---|---|---|
| | Placebo | 45 mg | 90 mg | | 135 mg | TAK-079 | |
| | (N = 5) | (N = 6) | (N = 6) | | (N = 5) | (N =17) | |
| Any TEAE (% of subjects) | 3 (60.0) | 4 (66.7) | 6 | (100.0) | 2 (40.0) | 12 | (70.6) |
| Grade 3 or higher TEAE | 0 | 0 | 0 | | 0 | 0 | |
| Drug-related TEAE (% of subjects) | 1 (20.0) | 1 (16.7) | 1 | (16.7) | 1 (20.0) | 3 | (17.6) |

| Drug-related grade 3 or higher | 0 | 0 | 0 | 0 | 0 | |
|---|---|---|---|---|---|---|
| TEAE | | | | | | |
| Treatment-emergent SAEs (% of | 0 | 1 (16.7) | 0 | 1 (20.0) | 2 | (11.8) |
| subjects) | | | | | | |
| Drug-related treatment- | 0 | 0 | 0 | 0 | 0 | |
| emergent SAEs | | | | | | |
| TEAEs resulting in study drug dose | 0 | 1 (16.7) | 0 | 1 (20.0) | 2 | (11.8) |
| modification^{a} (% of subjects) | | | | | | |
| TEAEs resulting in study drug | 0 | 0 | 0 | 1 (20.0) | 1 | (5.9) |
| discontinuation (% of subjects) | | | | | | |

| | | | | | |
|---|---|---|---|---|---|
| Deaths | 0 | 0 | 0 | 0 | 0 |

SAE: serious adverse event; TEAE: treatment-emergent adverse event.
^{a}Dose modification includes dose interrupted and drug withdrawn.

The most frequent TEAEs (occurring in more than 2 subjects in TAK-079 total group) are presented by SOC and PT in Table 29.

TABLE 29
Frequent (N > 2) Nonserious TEAEs by SOC and PT (Safety Analysis Set)

| | Number (%) of Subjects | | | | |
|---|---|---|---|---|---|
| | Pooled | TAK-079 | TAK-079 | TAK-079 | Total |
| SOC | Placebo | 45 mg | 90 mg | 135 mg | TAK-079 |
| PT | (N = 5) | (N = 6) | (N = 6) | (N = 5) | (N = 17) |
| Subjects with at least 1 TEAE | 3 (60.0) | 4 (66.7) | 6 (100.0) | 2 (40.0) | 12 (70.6) |
| Gastrointestinal disorders | 1 (20.0) | 3 (50.0) | 0 | 0 | 3 (17.6) |
| Nausea | 1 (20.0) | 3 (50.0) | 0 | 0 | 3 (17.6) |
| Infections and infestations | 2 (40.0) | 2 (33.3) | 3 (50.0) | 1 (20.0) | 6 (35.3) |
| Urinary tract infection | 0 | 2 (33.3) | 1 (16.7) | 1 (20.0) | 4 (23.5) |

MedDRA: Medical Dictionary for Regulatory Activities; PT: Preferred Term; SOC: System Organ Class; TEAE: treatment-emergent adverse event.
PTs which occurred in more than 2 subjects in TAK-079 total group are included. A subject is counted once for each PT and SOC.
Adverse events are coded using MedDRA version 24.0.

### Analysis of AEs

TEAEs were infrequent in the study participants. The 2 most common TEAEs regardless of causality were nausea and urinary tract infection. No subjects in the placebo group and 4 subjects (23.5%) in the pooled TAK-079 group reported TEAEs of urinary tract infection and 1 subject (20.0%) in the placebo group and 3 subjects (17.6%) in the pooled TAK-079 group reported TEAEs of nausea (Table 31). All other TEAEs were reported by 2 subjects or less.

All TEAEs had a maximum intensity of CTCAE Grade 1 or Grade 2.

### Deaths, Other SAES, and Other Significant AES

### Listing of Deaths, Other SAEs, and Other Significant AEs Deaths

No deaths occurred during the study.

### Other SAEs

Two treatment-emergent SAEs occurred during the study: palpitations reported by a subject in the TAK-079 45 mg group and dyspnoea reported by a subject in the TAK-079 135 mg group (Table 30). Both treatment-emergent SAEs were CTCAE Grade 2, were judged to be not related to the study drug, and had an outcome of recovered/resolved. The event of palpitations did not result in any changes to study drug, the event of dyspnoea resulted in withdrawal of the study drug.

TABLE 30
Treatment-Emergent SAEs by SOC and PT (Safety Analysis Set)

| | Number (%) of Subjects | | | | |
|---|---|---|---|---|---|
| | Pooled | TAK-079 | TAK-079 | TAK-079 | Total |
| SOC | Placebo | 45 mg | 90 mg | 135 mg | TAK-079 |
| PT | (N = 5) | (N = 6) | (N = 6) | (N = 5) | (N = 17) |
| Subjects with at least 1 treatment- emergent | 0 | 1 (16.7) | 0 | 1 (20.0) | 2 (11.8) |
| SAE | | | | | |
| Cardiac disorders | 0 | 1 (16.7) | 0 | 0 | 1 (5.9) |
| Palpitations | 0 | 1 (16.7) | 0 | 0 | 1 (5.9) |
| Respiratory, thoracic and mediastinal | 0 | 0 | 0 | 1 (20.0) | 1 (5.9) |
| disorders | | | | | |
| Dyspnoea | 0 | 0 | 0 | 1 (20.0) | 1 (5.9) |

MedDRA: Medical Dictionary for Regulatory Activities; PT: Preferred Term; SAE: serious adverse event; SOC: System Organ Class.
A subject is counted once for each PT and SOC.
Adverse events are coded using MedDRA version 24.0.

### Other Significant AEs

The most frequent non-serious adverse events (AEs) are presented in Table 31.

TABLE 31
Most Frequent Non-Serious Adverse Events (AEs) by SOC and PT (Safety Analysis Set).

| | Number (%) of Subjects | | | | |
|---|---|---|---|---|---|
| | Pooled | TAK-079 | TAK-079 | TAK-079 | Total |
| SOC | Placebo | 45 mg | 90 mg | 135 mg | TAK-079 |
| PT | (N = 5) | (N = 6) | (N = 6) | (N = 5) | (N = 17) |
| Subjects with at least 1 non-serious AE | 3 (60.0%) | 4 (66.7%) | 6 (100%) | 2 (40.0%) | 12 (70.6%) |

Blood and lymphatic system disorders

| | | | | | |
|---|---|---|---|---|---|
| Lymphadenopathy | 0 | 1 (16.7%) | 0 | 0 | 1 (5.9%) |

Gastrointestinal disorders

| | | | | | |
|---|---|---|---|---|---|
| Nausea | 1 (20.0%) | 3 (50.0%) | 0 | 0 | 3 (17.6%) |
| Abdominal pain | 0 | 1 (16.7%) | 0 | 0 | 1 (5.9%) |
| Bezoar | 0 | 1 (16.7%) | 0 | 0 | 1 (5.9%) |
| Diarrhoea | 0 | 1 (16.7%) | 0 | 0 | 1 (5.9%) |
| Diverticulum intestinal | 0 | 1 (16.7%) | 0 | 0 | 1 (5.9%) |
| Flatulence | 0 | 1 (16.7%) | 0 | 0 | 1 (5.9%) |
| Impaired gastric emptying | 0 | 1 (16.7%) | 0 | 0 | 1 (5.9%) |
| Large intestinal polyp | 0 | 1 (16.7%) | 0 | 0 | 1 (5.9%) |

General disorders

| | | | | | |
|---|---|---|---|---|---|
| Pyrexia | 0 | 0 | 1 (16.7%) | 1 (20.0%) | 2 (11.8%) |
| Fatigue | 0 | 1 (16.7%) | 0 | 0 | 1 (5.9%) |

Infections and infestations

| | | | | | |
|---|---|---|---|---|---|
| Urinary tract infection | 0 | 2 (33.3%) | 1 (16.7%) | 1 (20.0%) | 4 (23.5%) |
| Cellulitis | 0 | 0 | 1 (16.7%) | 1 (20.0%) | 2 (11.8%) |
| Herpes zoster | 0 | 1 (16.7%) | 0 | 0 | 1 (5.9%) |
| Sinusitis | 0 | 0 | 1 (16.7%) | 0 | 1 (5.9%) |
| Vulvovaginal mycotic infection | 1 (20.0%) | 0 | 0 | 0 | 0 |
| Tooth infection | 1 (20.0%) | 0 | 0 | 0 | 0 |

Injury, poisoning and procedural complications

| | | | | | |
|---|---|---|---|---|---|
| Arthropod bite | 0 | 0 | 1 (16.7%) | 0 | 1 (5.9%) |
| Muscle strain | 1 (20.0%) | 0 | 1 (16.7%) | 0 | 1 (5.9%) |
| Skin laceration | 0 | 0 | 1 (16.7%) | 0 | 1 (5.9%) |
| Vaccination complication | 1 (20.0%) | 0 | 0 | 0 | 0 |

Musculoskeletal and connective tissue disorders

| | | | | | |
|---|---|---|---|---|---|
| Chondropathy | 0 | 0 | 1 (16.7%) | 0 | 1 (5.9%) |
| Joint swelling | 0 | 0 | 1 (16.7%) | 0 | 1 (5.9%) |
| Pain in extremity | 0 | 0 | 1 (16.7%) | 0 | 1 (5.9%) |
| Systemic lupus erythematosus | 1 (20.0%) | 0 | 0 | 0 | 0 |

Nervous system disorders

| | | | | | |
|---|---|---|---|---|---|
| Headache | 1 (20.0%) | 0 | 0 | 0 | 0 |

Psychiatric disorders

| | | | | | |
|---|---|---|---|---|---|
| Adjustment disorder with depressed | 0 | 0 | 1 (16.7%) | 0 | 1 (5.9%) |
| mood | | | | | |

Respiratory, thoracis and mediastinal disorders

| | | | | | |
|---|---|---|---|---|---|
| Dyspnoea | 0 | 0 | 0 | 1 (20.0%) | 1 (5.9%) |
| Oropharyngeal pain | 0 | 1 (16.7%) | 0 | 0 | 1 (5.9%) |
| Rhinorrhoea | 0 | 1 (16.7%) | 0 | 0 | 1 (5.9%) |

Skin and subcutaneous tissue disorders

| | | | | | |
|---|---|---|---|---|---|
| Rash | 0 | 0 | 1 (16.7%) | 0 | 1 (5.9%) |
| Systemic lupus erythematosus rash | 0 | 0 | 1 (16.7%) | 0 | 1 (5.9%) |

Vascular disorders

| | | | | | |
|---|---|---|---|---|---|
| Hypertension | 1 (20.0%) | 0 | 0 | 0 | 0 |

MedDRA: Medical Dictionary for Regulatory Activities; PT: Preferred Term; AE: adverse event; SOC: System Organ Class.
Safety analysis set included all subjects who were enrolled and received at least 1 dose of study drug.
A subject is counted once for cach PT and SOC.
Adverse events are coded using MedDRA version 24.0.
Frequency threshold for reporting other AEs: >5%

### Drug-Related TEAEs

In total, 1 subject (20.0%) in the placebo group and 3 subjects (17.6%) in the pooled TAK-079 group (1 from each dose group) experienced TEAEs considered by the investigator to be related to the study drug (Table 32).

TABLE 32
Drug-Related TEAEs by SOC and PT (Safety Analysis Set)

| | Number (%) of Subjects | | | | |
|---|---|---|---|---|---|
| | Pooled | TAK-079 | TAK-079 | TAK-079 | Total |
| SOC | Placebo | 45 mg | 90 mg | 135 mg | TAK-079 |
| PT | (N = 5) | (N = 6) | (N = 6) | (N = 5) | (N = 17) |
| Subjects with at least 1 TEAE | 1 (20.0) | 1 (16.7) | 1 (16.7) | 1 (20.0) | 3 (17.6) |
| Gastrointestinal disorders | 1 (20.0) | 1 (16.7) | 0 | 0 | 1 (5.9) |
| Nausea | 1 (20.0) | 1 (16.7) | 0 | 0 | 1 (5.9) |
| General disorders and | 0 | 0 | 0 | 1 (20.0) | 1 (5.9) |
| administration site conditions | | | | | |
| Pyrcxia | 0 | 0 | 0 | 1 (20.0) | 1 (5.9) |
| Infections and infestations | 0 | 1 (16.7) | 0 | 0 | 1 (5.9) |
| Herpes zoster | 0 | 1 (16.7) | 0 | 0 | 1 (5.9) |
| Urinary tract infection | 0 | 1 (16.7) | 0 | 0 | 1 (5.9) |
| Skin and subcutaneous tissue | 0 | 0 | 1 (16.7) | 0 | 1 (5.9) |
| disorders | | | | | |
| Rash | 0 | 0 | 1 (16.7) | 0 | 1 (5.9) |

MedDRA: Medical Dictionary for Regulatory Activities; PT: Preferred Term; SOC: System Organ Class; TEAE: treatment-emergent adverse event.
TEAEs are defined as adverse events that occur after the first does of study drug received in the treatment period and until the end of safety follow-up.
A subject is counted once for each PT and SOC. Events with missing relationships are included.
Adverse events are coded using MedDRA version 24.0.

### TEAEs Leading to Study Drug Discontinuation

One (20.0%) subject in the TAK-079 135 mg group reported a TEAE leading to study drug discontinuation. This event of dyspnoea is described further in Section of “Other SAEs”.

### TEAEs Leading to Dose Modification

Dose modification in this study consisted of either dose interrupted or drug withdrawn. No subjects in the placebo group and 2 subjects (11.8%) in the pooled TAK-079 group reported TEAEs leading to dose modification, including 1 TEAE of diarrhoea in the TAK-079 45 mg group and 1 TEAE of dyspnoea in the TAK-079 135 mg group. The event of dyspnoea was serious, also led to study drug discontinuation, and is described further in Section of “Other SAEs.”

### Analysis and Discussion of Deaths, Other SAEs, and Other Significant AEs

No subjects reported life-threatening or fatal AEs and 2 subjects reported serious TEAEs. No serious TEAEs were considered related to the study drug.

There were no cases of CRS or local injection site reactions in this study. From a systemic perspective, there was 1 CTCAE Grade 2 TEAE event of pyrexia in a subject in the TAK-079 135 mg group on the day after dose 1. This event was considered a hypersensitivity reaction. It was deemed to be related to study drug, did not result in a dose change, was treated with 1000 mg of paracetamol, and had an outcome of recovered/resolved.

### Serum Chemistry

There were no trends observed in the mean values or change from baseline in serum chemistry parameters. The higher mean serum creatinine observed in the TAK-079 135 mg group compared with the other treatment groups was driven by 1 subject who presented with high serum creatinine at baseline, which did not worsen but persisted throughout the study. This subject had underlying chronic kidney disease at study start.

There were no shifts by >1 CTCAE grade from baseline to postbaseline in any serum chemistry parameters.

There were no AEs related to serum chemistry parameters during the study.

### Hematology

There were no trends observed in the mean values or change from baseline in hematology parameters.

Shifts from baseline to postbaseline of >1 CTCAE grade in hematology parameters included shifts from baseline Grade 0 to worst postbaseline Grade 2 in blood neutrophils by 1 subject in the placebo group and 1 subject in the TAK-079 45 mg group, and shifts from baseline Grade 0 to worst postbaseline Grade 2 in blood lymphocytes by 1 subject in the TAK-079 45 mg group.

There were no AEs related to hematology parameters during the study.

### Urinalysis

There were no trends observed in the mean values or change from baseline in urinalysis parameters besides urine protein/creatinine ratio (UPCR) which was higher in the TAK 135 mg group compared with the other treatment groups. The higher mean UPCR in this treatment group was driven by 2 subjects who presented with high UPCR values at baseline that persisted throughout the study. One of the 2 subjects presented with a diagnosis of chronic kidney disease at study start.

Shifts from baseline to postbaseline of >1 CTCAE grade in urinalysis parameters included shifts from baseline Grade 0 to worst postbaseline Grade 2 in urine protein high by 1 subject in the placebo group, 2 subjects in the TAK-079 45 mg group, and 1 subject in the TAK-079 135 mg group:

There were no AEs related to urinalysis parameters during the study.

### Other Laboratory Results

No notable results were observed in other laboratory results.

### Vital Sins

There were no trends observed in the mean values or change from baseline in vital signs parameters.

### Physical Examinations

Physical examinations were symptom- and SLE disease-directed. Any significant clinical findings were noted as AEs (for AEs, see Section of “AEs”).

### ECGs

There were no trends observed in the mean values or change from baseline in ECG parameters.

At baseline, all subjects reported normal or not clinically significant abnormal ECG results. No subjects reported shifts to clinically significant abnormal postbaseline results at any time point.

### Safety Biomarkers

Biomarkers related to safety were based on A167 QTBNK flow cytometric analyses. CD3⁺ total T cells, CD8⁺ cytotoxic T cells, CD4⁺ helper T cells, B cells, and monocytes were examined for safety evaluations related to the potential depletion of immune cell populations.

The overall effect of TAK-079 on the cell counts for these cell types was modest and did not appear to be different between doses. CD3⁺ total and CD4⁺ helper T cell, B cells, and monocytes showed mean reductions of less than 20.0% decrease from baseline across the pooled TAK-079 group throughout the study. CD8⁺ cytotoxic T cells showed maximum mean reduction of 32.8% decrease from baseline, which was observed on Day 2 after the first dose and was transient as cell counts returned to baseline by predose on Day 22. Placebo group did not show any appreciable depletion for any of the aforementioned cell populations.

### Pregnancy

No pregnancies were reported during the study.

### Safety Conclusions

Overall, TAK-079 was well tolerated. There were no substantial imbalances in AEs between the treatment groups and no dose-dependent effects or safety concerns were identified.

Seventeen subjects were exposed to TAK-079 during the study.

The AE events reported are balanced across the placebo and treatment groups and consistent with the reported safety profile of the individual agents (hydroxychloroquine, prednisone, gabapentin and mycophenolic acid). Infections and cytopenias did not represent a clinical problem in this study.

TEAEs of urinary tract infection and nausea were reported by 4 subjects each; all other TEAEs were reported by 2 subjects or less.

All TEAEs had a maximum intensity of CTCAE Grade 1 or Grade 2. There were no life-threatening or fatal AEs.

Two subjects reported treatment-emergent SAEs, including palpitations reported by a subject in the TAK-079 45 mg group and dyspnoea reported by a subject in the TAK-079 135 mg group. Neither was related to the study drug. The event of dyspnoea resulted in withdrawal of the study drug. In total, 4 subjects (1 from each treatment group) experienced TEAEs considered by the investigator to be related to study drug.

There was 1 case of hypersensitivity reaction and no cases of cytokine release syndrome or injection site reactions.

There were no clinically significant results related to laboratory evaluations, vital signs, or ECGs.

The overall effects of TAK-079 on the cell counts for CD3+ total T cells, CD8+ cytotoxic T cells, CD4+ helper T cells, B cells, and monocytes were modest and did not appear to be different between doses.

Overall, TAK-079 was well tolerated. There were no substantial imbalances in AEs between the treatment groups and no dose-dependent effects or safety concerns were identified.

### DISCUSSION AND OVERALL CONCLUSIONS

### Discussion

The primary objective of this study was to evaluate the safety and tolerability of TAK-079 in subjects with SLE. To this end, TAK-079 was well tolerated and no safety concerns were identified in this study. The TAK-079 safety profile was consistent with that observed in the first-in-human study in healthy volunteers (TAK-079_101). There were no substantial imbalances in AEs between the treatment groups. All TEAEs had a maximum intensity of CTCAE Grade 1 or Grade 2. Four subjects (1 from each treatment group) experienced nonserious TEAEs considered related to study drug by the investigator. Neither of the 2 reported treatment-emergent SAEs were considered to be related. The most commonly reported AEs were urinary tract infection and nausea, each reported by a total of 4 subjects. There were no cases of CRS or local injection site reactions in this study and 1 case of hypersensitivity reaction. The safety dose holds were balanced across treatment groups, with no dose-dependent trend for increased safety-related dose holds among TAK-079-treated groups.

The secondary objective of the study was to assess PK, PD and immunogenicity of TAK-079 in subjects with SLE. TAK-079 exposure appeared to increase in a greater than dose proportional manner over the tested dose range, consistent with observations from the first-in-human study (TAK-079-101) and nonclinical studies (TAK-079-10666). Apparent nonlinearity in TAK-079 PK is in line with target-mediated drug disposition that is often observed for mAb and arises from binding to an abundant cellular receptor, such as CD38. Peak concentrations were generally observed at 108 hours (4.5 days) after dose, which is in agreement with expectations for SC drug administration. Other PK parameters were not evaluated in this study due to limited data. Incomplete concentration-time profiles for a majority of subjects also precluded proper assessment of drug accumulation after multiple dose administration and determination of ADA impact on exposure. Overall, there was a substantial variability in serum TAK-079 concentrations, and interpretation of PK data was confounded by dose holds, premature discontinuation, and excluded samples.

Target engagement of TAK-079 on CD38 antigen was evaluated via receptor occupancy on CD38+ NK cells. While NK cells are not the target cells responsible for production of autoantibodies, they serve as a surrogate PD marker due to their abundance in peripheral blood and high expression of the receptor. Target engagement by TAK-079 on CD38+ NK cells was dose-dependent, with peak occupancy coinciding with maximum CD38+ NK cell depletion. Receptor occupancy was not saturated at the doses tested and not sustained within the dosing interval. Return to baseline for receptor occupancy on CD38+ NK cells paralleled partial recovery of this cell population by the time of the next dose administration, which is likely beneficial for TAK-079's cytotoxicity against antibody-producing cells given the importance of these effector cells in driving ADCC effect.

Limited reductions in immunoglobulins were observed, with the best response seen for IgA. The greatest changes in IgG were observed at the 135 mg dose of TAK-079, with the other 2 dose levels showing minimal differences compared to the placebo group. Reductions in total immunoglobulins were generally not accompanied by changes in autoantibody concentrations on an individual level, which could be partially attributed to the overall modest immunoglobulin response. Furthermore, results were confounded by dose holds and small sample size.

The therapeutic hypothesis for investigating TAK-079 in SLE was based on targeting CD38-expressing cells, such as plasma cells and PBs, thus reducing the production of pathogenic autoantibodies. In this study subjects had to be positive for at least 1 of the following autoantibodies in order to be enrolled: anti-dsDNA, anti-SmD^{P}, ribonucleoprotein-70, Sjogrens SS-A, or Sjogrens SS-B. In addition, there were 5 other lupus-associated autoantibodies that were assessed. Reductions in autoantibodies for which patients were positive at baseline did not appear to be correlated with efficacy measures as assessed by the clinical rating scales, such as CLASI, SLEDAI-2K, and PGA of disease, which appears to be consistent with literature reports (Marks and Tullus (2012) Pediatr. Nephrol. 27(10): 1855-68; Pisetsky (2020) J Autoimmun. 110: 102356). Lack of apparent concordance could also be due to insufficient depletion of plasma cells resulting in limited reductions in immunoglobulins. In addition, given that each autoantibody may have distinct pathogenic contribution to disease activity (Dema and Charles (2016) Antibodies (Basel) 5(1): 2) and a number of subjects were positive for more than 1 autoantibody, a more uniform suppression of all autoantibodies in a given patient may be required to observe a correlation between autoantibody reduction and clinical improvement. Clinical response in the absence of PD effects in some subjects warrants investigation into the potential contribution of additional mechanisms not related to antibody-producing target cells to gain better insight into the link between the downstream pharmacology of targeting CD38 and clinical efficacy.

Overall, PD results suggest that the dosing regimen of TAK-079 in SLE could be potentially optimized, such that higher dose and/or more frequent dosing schedule may result in more robust PD effects. The extent of depletion observed for CD38-expressing cells in peripheral blood (i.e., PBs and NK cells) was consistent with results from the first-in-human study (TAK-079_101), which informed the dose selection for the present study. However, the magnitude of reduction in total immunoglobulins in SLE was less substantial than in healthy subjects, which may be explained by altered immune landscape in disease state.

The incidences of treatment-emergent ADA and pre-existing ADA were 23.8%, respectively, resulting in an overall ADA prevalence of 47.6% in this study. No apparent association between immunogenicity and PK/PD, efficacy, or safety was observed. The relatively high ADA prevalence in this study may be ascribed to the active immune status of patients with SLE autoimmune disease (Faustini et al. (2021) Arthritis Res. Ther. 23(1): 211-23). In addition, interruptions in scheduled drug administration could have contributed to ADA development: 3 of 5 subjects with treatment-emergent ADA response had titers detected after TAK-079 dose hold. High frequency of ADA formation in autoimmune patients has been observed in studies with biologics where treatment was intermittent or included a prolonged drug holiday (Atiqi et al. (2020) Front. Immunol. 11: 312). Immunogenicity results may also be partially attributed to low doses investigated in the present study, which have been associated with higher risk of ADA response (Mok et al. (2016) Expert Opin. Biol. Ther. 16(2): 201-11).

The exploratory objective of this study was to assess the effects of repeated administration of TAK-079 on disease activity using various clinical rating scales for SLE. The biggest limitation of the study with respect of this objective was that the study was not powered to assess significance in clinical scores across groups. In addition to this, the COVID-19 pandemic resulted in a high number of discontinuations and missed doses, further reducing the number of available subjects for efficacy assessments within each cohort.

An additional limitation with respect of efficacy exploratory endpoint was the relatively high placebo response observed in this study. This is not an uncommon phenomenon in SLE studies due the permitted concomitant use of background therapy, administration of rescue medications, inadequate clinical rating scales, reporter bias, and heterogeneity of the patient population (Mahieu et al. (2016) Lupus 25(10): 1122-40). The use of rescue therapies can both interfere with measuring the true effect of the investigational drug and inflate placebo response in SLE clinical trials (Mahieu et al. (2016) Lupus 25(10): 1122-40). Although the study was designed to recruit subjects who had not responded adequately to standard SLE background therapy treatment and had not recently had an acute flare that was moderate to severe in nature, all subjects recruited to this study were nevertheless receiving stable background therapy for SLE for the duration of the study. While there were no additions to background medications during the treatment period or increases in doses of ongoing background medications, patients on study were receiving a variety of different SLE medications and doses, making it challenging to distinguish the true effect of the investigational drug from the effect of ongoing background medications. It is therefore plausible that the use of stable background medication contributed to the efficacy results in this study, in which a mild to moderate improvement of SLE symptoms was observed in the placebo group regardless of the assessment tool. The positive results observed in the placebo group may be further supported by the use of slightly higher mean corticosteroids doses in this group compared to the 90 mg and 135 mg TAK-079 groups.

There is a lack of consensus among drug developers on the best way to conduct studies investigating efficacy in SLE. Currently available and most commonly used outcome measures such as the SLEDAI-2K were devised mostly from observational data sets, not prospective randomized clinical studies. As such, they have not been optimally designed to capture multi-organ clinical responses in a heterogenous SLE patient population (Mahieu et al. (2016) Lupus 25(10): 1122-40). The clinical rating scales employed in this study have different strengths and weaknesses. The SLEDAI-2K is an effective tool to measure changes in global disease activity but is not sensitive to worsening or improvement in individual organ systems (Mahieu et al. (2016) Lupus 25(10): 1122-40; Ohmura (2021) Mod. Rheumatol. 31(1): 20-8). The CLASI is a well-validated tool to assess cutaneous aspects of the disease beyond the typical “rash” which demonstrates high specificity but low sensitivity (Klein et al. (2011) Arch. Dermatol. 147(2): 203-8); however, having no minimum CLASI requirement as part of study and a high number of patients with CLASI <5 limited the usefulness of this scale during this study and led to a floor effect. The evaluation of the 44-joint assessment scale was aimed at a more accurate characterization of the joint involvement and associated treatment benefit on joints. However, this scale was originally developed for patients with rheumatoid arthritis and has not been validated in patients with SLE. Lastly, while the PGA is considered to be the most comprehensive disease activity index (Ohmura (2021) Mod. Rheumatol. 31(1): 20-8), in this study it demonstrated poor sensitivity with approximately 70% of study patients meeting responder criteria across the treatment groups; of these, 100% of placebo study patients met PGA responder criteria at the end of treatment.

In future studies, utilization of a more severe SLE patient population with a specific clinical presentation would be more appropriate at assessing efficacy and/or proof of mechanism. Inclusion of patients with higher baseline SLEDAI-2K scores and lupus nephritis may facilitate validation of the mechanism of actions of TAK-079 at decreasing anti-dsDNA antibodies, as shown in another SLE investigation with an anti-CD38 antibody (Ostendorf et a. (2020) N. Engl. J. Med. 383(12): 1149-55). Additionally, assessment of more targeted biomarkers of disease for cutaneous lupus such as type-I interferon gene signatures would provide better mechanistic understanding of the trend observed among patients with higher CLASI scores at baseline. Requiring a minimum CLASI score in future studies may also aid at minimizing the floor effect observed in this study and therefore aid in assessing whether patients with more severe cutaneous disease may effectively benefit from TAK-079 treatment.

While a potential trend for more profound CLASI response in patients with higher baseline scores was identified, there was a lack of relevant PD effects supporting the observed clinically relevant improvement in cutaneous manifestations. This warrants further investigation into the potential contribution of additional mechanisms of TAK-079 not related to antibody-producing target cells. More targeted in vitro and in vivo investigations are needed to further assess if TAK-079 may elicit clinical benefit in patients with severe SLE and/or patients with moderate to severe cutaneous lupus.

Clinical response (particularly in CLASI score) in the absence of immunoglobulin reductions in some subjects (FIG. 6B) warranted investigation into consequences of CD38 inhibition that are unrelated to antibody-producing target cells. CyTOF analysis identified immune landscape changes post-treatment, including reductions in Bregs, pDCs, and NK cells. Changes in upstream regulatory populations may have significant impact, both on effector cells (e.g., pDC loss and altered type I IFN signaling) and more broadly on immune landscape. To this end, unbiased cluster analysis identified (among others) two clusters representing populations with features of effector CD4 and CD8 T cells. Both populations appear to harbor a treatment effect, although this was most pronounced only at the highest tested dose.

Assessment of more targeted biomarkers for cutaneous lupus was undertaken to gain a mechanistic understanding of profound CLASI responses. These results showed a reduction in Type I IFN gene signature from whole blood RNA. While this suggest a systemic reduction in inflammatory response, it cannot be directly linked to the cutaneous findings. Skin biopsies were not performed in this trial, precluding evaluation of tissue IFN expression and immune cells implicated in its production (such as plasmacytoid dendritic cells and keratinocytes).

### CONCLUSIONS

TAK-079 in combination with background therapy was administered to 17 patients with moderate to severe SLE. The investigational treatment was well tolerated and no safety concerns were identified. Study drug exposure was balanced but low across treatment groups; the majority of missed doses were due to protocol prespecified sponsor-driven safety holds and the COVID-19 pandemic.

TAK-079 exposures in patients with SLE were nonlinear at the doses tested, which is likely due to the high affinity binding of the antibody to the CD38 target. Decreases in total immunoglobulins and autoantibodies were modest at the doses tested and did not appear to be correlated with each other or with clinical response. PD results suggest that the dosing regimen of TAK-079 in SLE could be potentially optimized, such that a higher dose and/or more frequent dosing schedule may result in more sustained target engagement and more substantial changes in downstream pharmacology.

Although the overall ADA prevalence in this study was relatively high, with approximately 24% of patients positive for ADA at baseline, no apparent association between immunogenicity and PK/PD, efficacy, or safety was observed.

Clinical efficacy was an exploratory endpoint and the study was not powered to provide definitive efficacy conclusions. No observable differences from placebo were observed either longitudinally or in responder analyses for any of the clinical rating scales.

Overall, data from this study suggest mezagitamab has a favorable safety profile, expected pharmacodynamic effects, and favorable mechanistic data supporting continued investigation in autoimmune diseases.

In conclusion, the study met the primary safety endpoint and TAK-079 was well tolerated at all doses tested in subjects with moderate to severe SLE.

### Example 5: Tak-079 Antibody Structure

This example provides a summary of the TAK-079 antibody structure characterization. The TAK-079 amino acid sequence is detailed in Table 2. Disulfide bridge locations are detailed in Table 33.

TABLE 33
Location of the Disulfide Bridges

| | | | | |
|---|---|---|---|---|
| Intra-H | 22-96 | 150-206 | 267-327 | 373-431 |
| | 22″-96″ | 150″-206″ | 267″-327″ | 373″-431″ |
| Intra-L | 22′-89′ | 138′-197′ | | |
| | 22″′-89″′ | 138″′-197″′ | | |
| Inter H-L | 226-215′ | 226″′-215″′ | | |
| Inter H-H | 232-232″ | 235-235″ | | |

Details of Glycosylation (location and types of sugar residues): The HC contains a consensus asparagine-linked (N-linked) glycosylation site at residue Asn303 (303 and 303″). Major species have been tentatively identified as GOF (asialo-, agalacto, core fucosylated biantennary glycan), G0 (asialo-, agalacto, biantennary glycan), M5 (oligomannose 5), and G1F (asialo-, monogalacto, core fucosylated biantennary glycan).

LC N-terminal glutamine cyclization to G1p (5-oxoproline, pyroglutamic acid).

$$L VL Q 1 > Glp ( {1}^{′} , {1}^{″′} ) .$$

C-terminal clipping: H CHS K2: 453, 453″.

The precursor nucleotide sequence is detailed in Table 34.

TABLE 34
Precursor Nucleotide Sequence

| | |
|---|---|
| Heavy | gaggtgcagctgttggagtctgggggaggcttggtacagcctggggggtccctgagactc |
| Chain | tcctgtgcagcctctggattcacctttgatgattatggcatgagctgggtccgccaagct |
| | ccagggaaggggctggagtgggtctcggatattagttggaatggcggtaagacgcactat |
| | gtggactccgtgaagggccaattcaccatctccagagacaattccaagaacacgctgtat |
| | ctgcaaatgaacagcctgagagccgaggacactgccgtgtattactgtgcgcgggggagc |
| | ttattccatgatagtagtggtttttacttcgggcactggggccagggtacactggtcacg |
| | gtgagctcagctagcacaaagggcccatcggtcttccccctggcaccctcctccaagagc |
| | acctctgggggcacagcggccctgggctgcctggtcaaggactacttccccgaaccggtg |
| | acggtgtcgtggaactcaggcgccctgaccagcggcgtgcacaccttcccggctgtccta |
| | cagtcctcaggactctactccctcagcagcgtggtgaccgtgccctccagcagcttgggc |
| | acccagacctacatctgcaacgtgaatcacaagcccagcaacaccaaggtggacaagaga |
| | gttgagcccaaatcttgtgacaaaactcacacatgtccaccgtgcccagcacctgaactc |
| | ctggggggaccgtcagtcttcctcttccccccaaaacccaaggacaccctcatgatctcc |
| | cggacccctgaggtcacatgcgtggtggtggacgtgagccacgaagaccctgaggtcaag |
| | ttcaactggtacgtggacggcgtggaggtgcataatgccaagacaaagccgcgggaggag |
| | cagtacaacagcacgtaccgtgtggtcagcgtcctcaccgtcctgcaccaggactggctg |
| | aatggcaaggagtacaagtgcaaggtctccaacaaagccctcccagcccccatcgagaaa |
| | accatctccaaagccaaagggcagccccgagaaccacaggtgtacaccctgcccccatcc |
| | cgggaggagatgaccaagaaccaggtcagcctgacctgcctggtcaaaggcttctatccc |
| | agcgacatcgccgtggagtgggagagcaatgggcagccggagaacaactacaagaccacg |
| | cctcccgtgctggactccgacggctccttcttcctctatagcaagctcaccgtggacaag |
| | agcaggtggcagcaggggaacgtcttctcatgctccgtgatgcatgaggctctgcacaac |
| | cactacacgcagaagagcctctccctgtccccgggtaaa |
| | (SEQ ID NO: 14) |
| Light | cagtctgtgctgactcagccaccctcagcgtctgggacccccgggcagagggtcaccatc |
| Chain | tcctgctctggaagcagctccaacattggggataattatgtatcctggtatcagcagctc |
| | ccaggaacggcccccaaactcctcatctatagggatagtcagcggccctcaggggtccct |
| | gaccgattctctggctccaagtctggcacctcagcctccctggccatcagtgggctccgg |
| | tccgaggatgaggctgattattactgccagtcctatgacagcagcctgagtggttcggta |
| | ttcggcggaggaaccaagctgacggtcctaggtcagcccaaggccaaccccactgtcact |
| | ctgttcccgccctcctctgaggagctccaagccaacaaggccacactagtgtgtctgatc |
| | agtgacttctacccgggagctgtgacagtggcctggaaggcagatggcagccccgtcaag |
| | gcgggagtggagaccaccaaaccctccaaacagagcaacaacaagtacgcggccagcagc |
| | tacctgagcctgacgcccgagcagtggaagtcccacagaagctacagctgccaggtcacg |
| | catgaagggagcaccgtggagaagacagtggcccctacagaatgttca |
| | (SEQ ID NO:15) |

### Substance Characterization

### Molecular Weight

The predicted molecular weight of the unmodified antibody based on the DNA sequence is 144,421 Daltons (Da). Molecular weight of the major species of intact antibody determined by ESI-MS are approximately 147,280 Da.

CDR-IMGT (closest V, J, and C genes an alleles) are detailed in Table 35.

IG class/subclass: IgG1.

Species/Taxonomy related structure: Human.

Antigen target: CD38.

Expression system: CHO-K1SV.

Clone name(s): M3C6.

TABLE 35
CDR-IMGT (Closest V, J and C genes and alleles)

| | | | |
|---|---|---|---|
| Heavy | V domain | Homo sapiens | IGHV3-23*01 1 VH 582 88.8 |
| Chain | J domain | Homo sapiens | IGHJ1*01 93.3% |
| | C domain | Homo sapiens | IGHG1*03 CH1 100.0% |
| | | Homo sapiens | IGHG1*01 CH2 100.0% |
| | | Homo sapiens | IGHG1*03 CH3 100.0% |
| Light | V domain | Homo sapiens | IGLV1-47*01 V-LAMBDA 91.8% |
| Chain | J domain | Homo sapiens | IGLJ2*01 100.0% |
| | C domain | Homo sapiens | IGLC1*02 C-LAMBDA 100.0% |

### INCORPORATION BY REFERENCE

The contents of all cited references (including literature references, patents, patent applications, and websites) that may be cited throughout this application are hereby expressly incorporated by reference in their entirety for any purpose, as are the references cited therein, to the same extent as if each individual reference was specifically and individually indicated to be incorporated by reference in its entirety for any purposes.

### EQUIVALENTS

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein. Modifications for carrying out the invention that are obvious to persons of skill in the art are intended to be within the scope of the appended claims.

We claim:

1. A method of treating moderate or severe systemic lupus erythematosus (SLE) in a subject, the method comprising administering to the subject an isolated human anti-CD38 antibody or antigen binding fragment thereof,
wherein the antibody or antigen binding fragment thereof comprises:
a variable heavy chain region comprising a CDR1 having the amino acid sequence of SEQ ID NO:3, a CDR2 having the amino acid sequence of SEQ ID NO:4, and a CDR3 having the amino acid sequence of SEQ ID NO:5; and
a variable light chain region comprising a CDR1 having the amino acid sequence of SEQ ID NO:6, a CDR2 having the amino acid sequence of SEQ ID NO:7, and a CDR3 having the amino acid sequence of SEQ ID NO:8; and
wherein the antibody or antigen binding fragment thereof is subcutaneously administered in a dosage of from about 40 milligrams to about 140 milligrams.

2. A method of reducing the level of plasmablasts and/or plasma cells in a subject diagnosed with moderate or severe SLE, the method comprising administering to the subject an isolated human anti-CD38 antibody or antigen binding fragment thereof,
wherein the antibody or antigen binding fragment thereof comprises:
a variable heavy chain region comprising a CDR1 having the amino acid sequence of SEQ ID NO:3, a CDR2 having the amino acid sequence of SEQ ID NO:4, and a CDR3 having the amino acid sequence of SEQ ID NO:5; and
a variable light chain region comprising a CDR1 having the amino acid sequence of SEQ ID NO:6, a CDR2 having the amino acid sequence of SEQ ID NO:7, and a CDR3 having the amino acid sequence of SEQ ID NO:8; and
wherein the antibody or antigen binding fragment thereof is subcutaneously administered in a dosage of from about 40 milligrams to about 140 milligrams.

3. A method of reducing the level of immunoglobulin(s) in a subject diagnosed with moderate or severe SLE, the method comprising administering to the subject an isolated human anti-CD38 antibody or antigen binding fragment thereof,
wherein the antibody or antigen binding fragment thereof comprises:
a variable heavy chain region comprising a CDR1 having the amino acid sequence of SEQ ID NO:3, a CDR2 having the amino acid sequence of SEQ ID NO:4, and a CDR3 having the amino acid sequence of SEQ ID NO:5; and
a variable light chain region comprising a CDR1 having the amino acid sequence of SEQ ID NO:6, a CDR2 having the amino acid sequence of SEQ ID NO:7, and a CDR3 having the amino acid sequence of SEQ ID NO:8; and
wherein the antibody or antigen binding fragment thereof is subcutaneously administered in a dosage of from about 40 milligrams to about 140 milligrams.

4. The method of claim 3, wherein the immunoglobulin is IgA, IgG and/or IgM.

5. A method of reducing the level of one or more autoantibodies in a subject diagnosed with moderate or severe SLE, the method comprising administering to the subject an isolated human anti-CD38 antibody or antigen binding fragment thereof,
wherein the antibody or antigen binding fragment thereof comprises:
a variable heavy chain region comprising a CDR1 having the amino acid sequence of SEQ ID NO:3, a CDR2 having the amino acid sequence of SEQ ID NO:4, and a CDR3 having the amino acid sequence of SEQ ID NO:5; and
a variable light chain region comprising a CDR1 having the amino acid sequence of SEQ ID NO:6, a CDR2 having the amino acid sequence of SEQ ID NO:7, and a CDR3 having the amino acid sequence of SEQ ID NO:8; and
wherein the antibody or antigen binding fragment thereof is subcutaneously administered in a dosage of from about 40 milligrams to about 140 milligrams.

6. The method of claim 5, wherein the one or more autoantibodies is selected from the group consisting of anti-dsDNA, anti-SmD^{P}, beta-2 glycoprotein 1 IgM, ribonucleoprotein-70, Sjogrens SS-A and Sjogrens SS-B.

7. The method of any one of the preceding claims, wherein the subject is diagnosed with severe SLE.

8. The method of any one of the preceding claims, wherein the antibody or antigen binding fragment thereof further comprises one or more engineered glycoforms, wherein the engineered glycoform comprises glycosylation of one or more polypeptides, optionally wherein the glycosylation is N-linked glycosylation or O-linked glycosylation, and optionally wherein the glycosylation is N-linked glycosylation.

9. The method of any one of the preceding claims, wherein the variable heavy chain region of the antibody or antigen binding fragment thereof comprises an amino acid sequence having an identity of at least 90% to SEQ ID NO:9, and/or the variable light chain region of the antibody or antigen binding fragment thereof comprises an amino acid sequence having an identity of at least 90% to SEQ ID NO:10;
optionally wherein the variable heavy chain region of the antibody or antigen binding fragment thereof comprises an amino acid sequence having an identity of at least 95% to SEQ ID NO:9, and/or the variable light chain region of the antibody or antigen binding fragment thereof comprises an amino acid sequence having an identity of at least 95% to SEQ ID NO:10;
optionally wherein the variable heavy chain region of the antibody or antigen binding fragment thereof comprises an amino acid sequence having an identity of at least 99% to SEQ ID NO:9, and/or the variable light chain region of the antibody or antigen binding fragment thereof comprises an amino acid sequence having an identity of at least 99% to SEQ ID NO:10;
optionally wherein the heavy chain of the antibody or antigen binding fragment thereof comprises an amino acid sequence having an identity of at least 95% to SEQ ID NO:11; and/or the light chain of the antibody or antigen binding fragment thereof comprises an amino acid sequence having an identity of at least 95% to SEQ ID NO:12;
optionally wherein the isolated antibody or antigen binding fragment thereof interacts with at least K121, F135, Q139, D141, E239, W241, C275, K276, F284, P291 and E292 of SEQ ID NO:1 and SEQ ID NO:2, based on human sequence numbering;
optionally wherein the isolated antibody or antigen binding fragment thereof binds to human CD38 (SEQ ID NO:1) with a KD of 10⁻⁸ M or a greater affinity, and wherein the affinity is measured by a standard Biacore assay;
optionally wherein the variable heavy chain region comprises SEQ ID NO:9 and the variable light chain region comprises SEQ ID NO:10; and
optionally wherein the isolated antibody or antigen binding fragment thereof comprises a heavy chain as set forth in SEQ ID NO:11 and a light chain as set forth in SEQ ID NO:12.

10. The method of any one of the preceding claims, wherein the antibody or antigen binding fragment thereof further comprises an Fc domain;
optionally wherein the Fc domain is a human Fc domain or a variant Fc domain; and
optionally wherein the isolated antibody or antigen binding fragment is a human IgG antibody, optionally wherein the human IgG antibody is a human IgG1 antibody.

11. The method of any one of the preceding claims, wherein the subject receives background SLE medication(s), optionally wherein the background SLE medication(s) is selected from the group consisting of immunosuppressants, steroids and immunoglobulin; and optionally wherein the background SLE medication(s) is selected from the group consisting of hydroxychloroquine, hydroxychloroquine sulfate, prednisone, methylprednisolone, gabapentin, mycophenolate mofetil, and/or mycophenolic acid.

12. The method of claim 11, wherein the background SLE medication(s) is administered in combination with the antibody or antigen binding fragment thereof.

13. The method of any one of the preceding claims, wherein administering the antibody or antigen binding fragment thereof results in less than 10% incidence of grade 3 or 4 of one or more treatment-related adverse events (TRAEs) or treatment-emergent adverse events (TEAEs);
optionally wherein the TRAEs or TEAEs are selected from the group consisting of gastrointestinal disorders, nausea, infestations, pyrexia, Herpes Zoster, urinary tract infection, skin and cutaneous tissue disorders, headache, fever, chills/rigors, vomiting, diarrhea, arthralgia, myalgia, hypotension, respiratory, thoracic and mediastinal disorders, thrombocytopenia, leukopenia, lymphopenia, cardiac disorders, palpitations, and dyspnea; and
optionally wherein administering the antibody or antigen binding fragment thereof results in one or more TRAEs or TEAEs having a maximum intensity of Common Terminology Criteria for Adverse Events (CTCAE) Grade 1 or Grade 2.

14. The method of any one of the preceding claims, wherein the antibody or antigen binding fragment thereof is administered in a dosage selected from the group consisting of about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, and about 140 mg; and
optionally wherein the antibody or antigen binding fragment thereof is administered in a dosage of about 45 mg, about 90 mg or about 135 mg.

15. The method of any one of the preceding claims, wherein the dosage is a dosage administered once every week, once every two weeks, once every three weeks or once every four weeks.

16. The method of any one of the preceding claims, wherein the antibody or antigen binding fragment thereof is administered in the form of a pharmaceutically acceptable composition, and optionally wherein the pharmaceutically acceptable composition comprises the isolated antibody or antibody fragment thereof and at least one pharmaceutically acceptable carrier, excipient or stabilizer.

17. The method of any one of the preceding claims, wherein the isolated antibody or antigen binding fragment thereof comprises a heavy chain as set forth in SEQ ID NO:11 and a light chain as set forth in SEQ ID NO:12; and wherein the antibody or antigen binding fragment thereof is subcutaneously administered once every 3 weeks for 12 weeks.

18. The method of any one of the preceding claims, wherein the isolated antibody or antigen binding fragment thereof is Mezagitamab.

19. A unit dosage form comprising an isolated antibody or antigen binding fragment thereof that comprises a variable heavy chain region comprising a CDR1 having the amino acid sequence of SEQ ID NO:3, a CDR2 having the amino acid sequence of SEQ ID NO:4, and a CDR3 having the amino acid sequence of SEQ ID NO:5; and
a variable light chain region comprising a CDR1 having the amino acid sequence of SEQ ID NO:6, a CDR2 having the amino acid sequence of SEQ ID NO:7, and a CDR3 having the amino acid sequence of SEQ ID NO:8;
wherein the isolated antibody or antigen binding fragment thereof binds to human CD38 (SEQ ID NO:1), and the unit dosage form is formulated for subcutaneous administration of the antibody or antigen binding fragment thereof at a dosage of from 40 milligrams to 140 milligrams in the treatment of moderate or severe systemic lupus erythematosus (SLE).

20. The unit dosage form of claim 19, wherein the unit dosage form is formulated for subcutaneous administration of the antibody or antigen binding fragment thereof in the treatment of severe SLE.

21. The unit dosage form of claim 19 or claim 20, wherein the antibody or antigen binding fragment thereof further comprises one or more engineered glycoforms, wherein the engineered glycoform comprises glycosylation of one or more polypeptides, optionally wherein the glycosylation is N-linked glycosylation or O-linked glycosylation, and optionally wherein the glycosylation is N-linked glycosylation.

22. The unit dosage form of any one of claims 19-21, wherein the variable heavy chain region of the antibody or antigen binding fragment thereof comprises an amino acid sequence having an identity of at least 90% to SEQ ID NO:9, and/or the variable light chain region of the antibody or antigen binding fragment thereof comprises an amino acid sequence having an identity of at least 90% to SEQ ID NO:10;
optionally wherein the variable heavy chain region comprises an amino acid sequence having an identity of at least 95% to SEQ ID NO:9, and/or the variable light chain region comprises an amino acid sequence having an identity of at least 95% to SEQ ID NO:10;
optionally wherein the variable heavy chain region comprises an amino acid sequence having an identity of at least 99% to SEQ ID NO:9, and/or the variable light chain region comprises an amino acid sequence having an identity of at least 99% to SEQ ID NO:10;
optionally wherein the heavy chain of the antibody or antigen binding fragment thereof comprises an amino acid sequence having an identity of at least 95% to SEQ ID NO:11, and/or the light chain of the antibody or antigen binding fragment thereof comprises an amino acid sequence having an identity of at least 95% to SEQ ID NO:12;
optionally wherein the isolated antibody or antigen binding fragment thereof interacts with at least K121, F135, Q139, D141, E239, W241, C275, K276, F284, P291 and E292 of SEQ ID NO:1 and SEQ ID NO:2, based on human sequence numbering;
optionally wherein the isolated antibody or antigen binding fragment thereof binds to human CD38 (SEQ ID NO:1) with a KD of 10⁻⁸ M or a greater affinity, and wherein the affinity is measured by a standard Biacore assay;
optionally wherein the variable heavy chain region comprises SEQ ID NO:9 and the variable light chain region comprises SEQ ID NO:10; and
optionally wherein the isolated antibody or antigen binding fragment thereof comprises a heavy chain as set forth in SEQ ID NO:11 and a light chain as set forth in SEQ ID NO:12.

23. The unit dosage form of any one of claims 19-22, wherein the isolated antibody or antigen binding fragment thereof further comprises an Fc domain, optionally wherein the Fc domain is a human Fc domain or a variant Fc domain; and
optionally wherein the isolated antibody or antigen binding fragment is a human IgG antibody,
optionally wherein the human IgG antibody is a human IgG1 antibody.

24. The unit dosage form of any one of claims 19-23, wherein the isolated antibody or antigen binding fragment thereof is used in combination with one or more background SLE medications, optionally wherein the background SLE medication(s) is selected from the group consisting of immunosuppressants, steroids and immunoglobulin; optionally wherein the background SLE medication(s) is selected from the group consisting of hydroxychloroquine, hydroxychloroquine sulfate, prednisone, methylprednisolone, gabapentin, mycophenolate mofetil, and/or mycophenolic acid; and optionally wherein the unit dosage form further comprises the one or more background SLE medications.

25. The unit dosage form of any one of claims 19-24, wherein administering the antibody or antigen binding fragment thereof results in less than 10% incidence of grade 3 or 4 of one or more treatment-related adverse events (TRAEs) or treatment-emergent adverse events (TEAEs);
optionally wherein the TRAEs or TEAEs are selected from the group consisting of gastrointestinal disorders, nausea, infestations, pyrexia, Herpes Zoster, urinary tract infection, skin and cutaneous tissue disorders, headache, fever, chills/rigors, vomiting, diarrhea, arthralgia, myalgia, hypotension, respiratory, thoracic and mediastinal disorders, thrombocytopenia, leukopenia, lymphopenia, cardiac disorders, palpitations, and dyspnea; and
optionally wherein administering the antibody or antigen binding fragment thereof results in one or more TRAEs or TEAEs having a maximum intensity of Common Terminology Criteria for Adverse Events (CTCAE) Grade 1 or Grade 2.

26. The unit dosage form of any one of claims 19-25, wherein the antibody or antigen binding fragment thereof is administered in a dosage selected from the group consisting of about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, and about 140 mg; and optionally wherein the antibody or antigen binding fragment thereof is administered in a dosage of about 45 mg, about 90 mg or about 135 mg.

27. The unit dosage form of any one of claims 19-26, wherein the dosage is a dosage administered once every week, once every two weeks, once every three weeks or once every four weeks.

28. The unit dosage form of any one of claims 19-27 further comprising at least one pharmaceutically acceptable carrier, excipient, or stabilizer.

29. The unit dosage form of any one of claims 19-28, wherein the isolated antibody or antigen binding fragment thereof comprises a heavy chain as set forth in SEQ ID NO:11 and a light chain as set forth in SEQ ID NO:12; and wherein the antibody or antigen binding fragment thereof is subcutaneously administered once every 3 weeks for 12 weeks.

30. The unit dosage form of any one of claims 19-29, wherein the isolated antibody or antigen binding fragment thereof is Mezagitamab.


SEQUENCE LISTING (15 sequences)

SEQ ID NO: 1 - protein, 300 aa, synthetic construct
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 2 - protein, 301 aa, synthetic construct
MANCEFSPVSGDKPCCRLSRRAQVCLGVCLLVLLILVVVVAVVLPRWRQQWSGSGTTSRFPETVLARCVKYTEVHPEMRHVDCQSVWDAFKGAFISKYPCNITEEDYQPLVKLGTQTVPCNKTLLWSRIKDLAHQFTQVQRDMFTLEDMLLGYLADDLTWCGEFNTFEINYQSCPDWRKDCSNNPVSVFWKTVSRRFAETACGVVHVMLNGSRSKIFDKNSTFGSVEVHNLQPEKVQALEAWVIHGGREDSRDLCQDPTIKELESIISKRNIRFFCKNIYRPDKFLQCVKNPEDSSCLSGI

SEQ ID NO: 3 - protein, 8 aa, synthetic construct
GFTFDDYG

SEQ ID NO: 4 - protein, 8 aa, synthetic construct
ISWNGGKT

SEQ ID NO: 5 - protein, 16 aa, synthetic construct
ARGSLFHDSSGFYFGH

SEQ ID NO: 6 - protein, 8 aa, synthetic construct
SSNIGDNY

SEQ ID NO: 7 - skipped

SEQ ID NO: 8 - protein, 10 aa, synthetic construct
QSYDSSLSGS

SEQ ID NO: 9 - protein, 135 aa, synthetic construct
EVQLLESGGGLVQPGGSLRLSCAASGFTFDDYGMSWVRQAPGKGLEWVSDISWNGGKTHYVDSVKGQFTISRDNSKNTLYLQMNSLRAEDTAVYYCARGSLFHDSSGFYFGHWGQGTLVTVSSASTKGPSVFPLA

SEQ ID NO: 10 - protein, 129 aa, synthetic construct
QSVLTQPPSASGTPGQRVTISCSGSSSNIGDNYVSWYQQLPGTAPKLLIYRDSQRPSGVPDRFSGSKSGTSASLAISGLRSEDEADYYCQSYDSSLSGSVFGGGTKLTVLGQPKANPTVTLFPPSSEEL

SEQ ID NO: 11 - protein, 453 aa, synthetic construct
EVQLLESGGGLVQPGGSLRLSCAASGFTFDDYGMSWVRQAPGKGLEWVSDISWNGGKTHYVDSVKGQFTISRDNSKNTLYLQMNSLRAEDTAVYYCARGSLFHDSSGFYFGHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 216 aa, synthetic construct
QSVLTQPPSASGTPGQRVTISCSGSSSNIGDNYVSWYQQLPGTAPKLLIYRDSQRPSGVPDRFSGSKSGTSASLAISGLRSEDEADYYCQSYDSSLSGSVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 13 - skipped

SEQ ID NO: 14 - DNA, 1359 bp, unidentified
  source 1..1359: mol_type = other DNA
gaggtgcagctgttggagtctgggggaggcttggtacagcctggggggtccctgagactctcctgtgcagcctctggattcacctttgatgattatggcatgagctgggtccgccaagctccagggaaggggctggagtgggtctcggatattagttggaatggcggtaagacgcactatgtggactccgtgaagggccaattcaccatctccagagacaattccaagaacacgctgtatctgcaaatgaacagcctgagagccgaggacactgccgtgtattactgtgcgcgggggagcttattccatgatagtagtggtttttacttcgggcactggggccagggtacactggtcacggtgagctcagctagcacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgtccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 15 - DNA, 648 bp, unidentified
  source 1..648: mol_type = other DNA
cagtctgtgctgactcagccaccctcagcgtctgggacccccgggcagagggtcaccatctcctgctctggaagcagctccaacattggggataattatgtatcctggtatcagcagctcccaggaacggcccccaaactcctcatctatagggatagtcagcggccctcaggggtccctgaccgattctctggctccaagtctggcacctcagcctccctggccatcagtgggctccggtccgaggatgaggctgattattactgccagtcctatgacagcagcctgagtggttcggtattcggcggaggaaccaagctgacggtcctaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttca